(19) United States
(12) Patent Application Publication
(10) Pub. No.: US 2024/0263173 A1
(43) Pub. Date: Aug. 8, 2024

(54) HIGH-THROUGHPUT PRECISION GENOME EDITING IN HUMAN CELLS

(71) Applicant: The Board of Trustees of the Leland Stanford Junior University, Stanford, CA (US)

(72) Inventors: Hunter B. Fraser, Stanford, CA (US); Bin Zhao, Stanford, CA (US); Shi-An Anderson Chen, Stanford, CA (US)

(73) Assignee: The Board of Trustees of the Leland Stanford Junior University, Stanford, CA (US)

(21) Appl. No.: 18/682,853

(22) PCT Filed: Aug. 10, 2022
(86) PCT No.: PCT/US2022/074751

§ 371 Date: Feb. 9, 2024

Related U.S. Application Data
(60) Provisional application No. 63/232,080, filed on Aug. 11, 2021.

(51) Int. Cl. C12N 15/11; C12N 15/85

(52) U.S. Cl. CPC C12N 15/11; C12N 15/85; C12N 2310/20; C12N 2830/55

(57) ABSTRACT

The disclosure provides compositions and methods for high-efficiency genome editing in mammals. In some aspects, the disclosure provides guide RNA-retron cassettes, RNA-guided nuclease (e.g., Cas9)-reverse transcriptase fusion protein encoding cassettes, and vectors comprising the cassettes. Also provided are mammalian host cells that have been transfected with the vectors. In other aspects, guide-retron donor DNA molecules are provided. In some other aspects, methods for genome editing in mammals and the screening of genetic loci are provided. In further aspects, methods and compositions are provided for the prevention or treatment of genetic diseases and for other applications. Kits for genome editing and screening are also provided.

### CROSS-REFERENCE TO RELATED APPLICATIONS

This application claims priority to U.S. Provisional Application No. 63/232,080, filed Aug. 11, 2021, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under grant number 1R01GM13422801 awarded by the National Institutes of Health. The Government has certain rights in the invention.

### BACKGROUND

Precise genome editing is a promising tool for identifying causal genetic variants and their function, generating disease models, and performing gene therapy, among other applications (1, 2). Traditionally, precise genome editing with scarless replacement of alleles or insertion of synthetic sequences requires in vitro delivery of DNA donors. However, it has proven challenging to induce cells to utilize donor DNA to conduct homology-directed repair (HDR), resulting in non-homologous end joining (NHEJ) repair, which is error-prone (3). To date, the most efficient donor delivery systems are in vitro synthetic DNA and viral vectors (4, 5). Synthetic DNA donors are delivered to cells directly via electroporation or by packaging them into particles without specifically targeting the nucleus, while viral vectors such as adeno-associated virus (AAV) are transduced to enter the nucleus (6-8). In both in vitro synthetic DNA and viral vector donor delivery, the donors are non-renewable after delivery and are depleted over time, decreasing editing after cell division in mitotic progeny. In addition, neither method scales well for multiplexed editing, which requires specific guide and donor combinations that can only happen by chance with bulk delivery. Finally, synthetic DNA and viral vector donor delivery is limited by cost and labor when scaling up for screening through tens of thousands of individual variants. Therefore, a biological solution enabling in nucleo donor generation would fundamentally improve the scalability and multiplexing capabilities for genomic knock-ins.

Retrons have been studied since the 1970s as bacterial genetic elements that encode unique features (9, 10), one of which is the production of multicopy single-stranded DNA (msDNA), which has been biochemically purified from retron-expressing cells (11). The minimal retron element consists of a contiguous cassette that encodes an RNA (msr-msd) and a reverse transcriptase (RT). The RT reverse transcribes the msd section to generate msDNA, a single-stranded DNA-RNA hybrid comprising the reverse-transcribed DNA covalently tethered to the non-reverse-transcribed RNA. Retron sequences are diverse among bacterial species but share similar RNA secondary structures (9). The RT recognizes the secondary structure of retron RNA hairpin loops in the msr region and subsequently initiates reverse transcription branching off of the guanosine residue flanking the self-annealed double-stranded DNA priming region (12, 13). This process has two properties that differentiate retrons from typical viral reverse transcriptases commonly used in biotechnology (9). First, the RT targets only the msr-msd from the same retron as its RNA template, providing specificity that may be useful for avoiding off-target reverse transcription (12). Second, the RNA template self-anneals intramolecularly in cis rather than requiring primers in trans to increase efficiency. Combining these two features allows cell-autonomous production of specific single-stranded donor DNA in the nucleus, circumventing the need for external donor delivery through chemical methods or viral vectors. Therefore, when coupled with targeted nucleases, retrons are promising biological sources for generating DNA donors for template-mediated precise genome editing.

Despite these advances, numerous challenges remain for the adaptation of retron-mediated genome editing to other systems, such as mammals, and challenges also remain for the efficient delivery of homologous donor templates for homologous repair-mediated genome editing. The present disclosure addresses these needs and provides additional advantages as well.

### BRIEF SUMMARY

In one aspect, the present disclosure provides a guide RNA (gRNA)-retron cassette for use in genomic editing in a mammalian cell comprising: (a) a gRNA coding region, wherein the target sequence of the gRNA is within a mammalian genetic locus; and (b) a retron region comprising: (i) an msr locus; (ii) a first inverted repeat sequence; (iii) an msd locus; (iv) a donor DNA template region located within the msd locus, wherein the donor DNA template comprises homology to one or more sequences within the mammalian genetic locus; and (v) a second inverted repeat sequence, wherein the gRNA coding region is upstream of the retron region in the cassette such that transcription of the cassette results in a transcript in which the gRNA is 5′ of the RNA transcribed from the retron region.

In some embodiments, the first inverted repeat sequence is located within the 5′ end of the msr locus. In some embodiments, the second inverted repeat sequence is located 3′ of the msd locus. In some embodiments, the retron region encodes an RNA molecule that is capable of self-priming reverse transcription by a reverse transcriptase (RT). In some embodiments, reverse transcription of the RNA molecule produces a hybrid molecule that comprises RNA and single stranded DNA (ssDNA), wherein the RNA within the hybrid molecule comprises the gRNA, wherein the ssDNA within the hybrid molecule comprises the donor DNA template, and wherein the gRNA and donor DNA template are covalently linked. In some embodiments, the donor DNA template comprises two homology arms, wherein each homology arm has at least about 70% to about 99% similarity to a portion of the mammalian genetic locus on either side of the gRNA target sequence.

In another aspect, the present disclosure provides a vector comprising any of the herein-described cassettes.

In some embodiments, the vector further comprises a promoter that is operably linked to the cassette. In some embodiments, the promoter is an RNA polymerase III (Pol III) promoter. In some embodiments, the msd locus comprises one or more sequence modifications to avoid pre-mature Pol III termination. In some embodiments, the one or more sequence modifications comprise single nucleotide substitutions. In some embodiments, the msd locus comprises a “TTTT” to “TTTc” or “TTTa” sequence modification in the stem region. In some embodiments, the msd locus further comprises a modification of a corresponding sequence in the opposite strand of the stem region for maintaining secondary structure. In certain embodiments, the modification of the corresponding sequence comprises a “GGAAA” to “GGgAA” sequence modification or a “GAAAA” to “GgAAA” sequence modification. In some embodiments, the msd locus further comprises a “TTTTTT” to “TTTETT” sequence modification downstream of the stem region. In particular embodiments, the msd locus comprises an Ec86 msd sequence.

In some embodiments, the vector further comprises a second cassette comprising a coding sequence for a fusion protein comprising an RNA-guided nuclease and a reverse transcriptase (RT). In some embodiments, the vector further comprises a second cassette comprising a coding sequence for a bicistronic polypeptide comprising an RNA-guided nuclease and a reverse transcriptase (RT), separated by a self-cleaving peptide. In some embodiments, the self-cleaving peptide is E2A (e.g., QCTNYALLKLAGDVESNPGP; SEQ ID NO:62), T2A (e.g., EGRGSLLTCGDVEENPGP; SEQ ID NO:63), P2A (e.g., ATNFSLLKQAGDVEENPGP; SEQ ID NO:64), or F2A (e.g. VKQTLNFDLLKLAGDVESNPGP; SEQ ID NO:65). In some instances, the E2A, T2A, P2A, or F2A self-cleaving peptide further comprises a linker sequence (e.g., GSG) at the N-terminal end of the peptide. In some embodiments, the coding sequence is codon optimized for mammalian cells. In some embodiments, the RNA-guided nuclease is Cas9 or Cpf1. In some embodiments, the vector comprises a promoter operably linked to the second cassette. In some embodiments, the promoter operably linked to the second cassette is an RNA polymerase II (Pol II) promoter.

In another aspect, the present disclosure provides a gRNA-msr-msd-donor RNA molecule for use in genomic editing in a mammalian cell comprising: (a) a guide RNA (gRNA), wherein the target sequence of the gRNA is within a mammalian genetic locus; and (b) a retron transcript comprising: (i) an msr region; (ii) a first inverted repeat sequence; (iii) an msd region; (iv) a donor DNA template coding region located within the msd region, wherein the encoded donor DNA template comprises homology to the mammalian genetic locus; and (v) a second inverted repeat sequence.

In some embodiments, the first inverted repeat sequence is located within the 5′ end of the msr region. In some embodiments, the second inverted repeat sequence is located 3′ of the msd region. In some embodiments, the retron transcript is capable of self-priming reverse transcription by a reverse transcriptase (RT). In some embodiments, the gRNA is 5′ of the retron transcript. In some embodiments, reverse transcription of the retron transcript produces a hybrid molecule that comprises RNA and single stranded DNA (ssDNA), wherein the RNA within the hybrid molecule comprises the gRNA, wherein the ssDNA comprises the donor DNA template, and wherein the gRNA and donor DNA template are covalently linked. In some embodiments, the donor DNA template coding region comprises sequences encoding two homology arms, wherein each homology arm has at least about 70% to about 99% similarity to a portion of the mammalian genetic locus on either side of the gRNA target sequence.

In another aspect, the present disclosure provides a method for modifying one or more target nucleic acids of interest at one or more target loci within a genome of a mammalian host cell, the method comprising: (a) transforming the mammalian host cell with any of the herein-described vectors; and (b) culturing the host cell or transformed progeny of the host cell under conditions sufficient for expressing from the vector a gRNA-msr-msd-donor RNA molecule, wherein the retron transcript within the gRNA-msr-msd-donor RNA molecule self-primes reverse transcription by a reverse transcriptase (RT) expressed by the host cell or the transformed progeny of the host cell, wherein at least a portion of the retron transcript is reverse transcribed to produce a hybrid molecule that comprises RNA and single stranded DNA (ssDNA), wherein the RNA within the hybrid molecule comprises the gRNA, wherein the ssDNA within the hybrid molecule comprises the donor DNA template, wherein the gRNA and donor DNA template are covalently linked, wherein the donor DNA template comprises homology to the one or more target loci and comprises sequence modifications compared to the one or more target nucleic acids, wherein the one or more target loci are cut by an RNA-guided nuclease expressed by the host cell or transformed progeny of the host cell, wherein the reverse transcriptase and the RNA-guided nuclease are present within a single fusion protein or a bicistronic polypeptide separated by a self-cleaving peptide, wherein the site of cutting by the RNA-guided nuclease is determined by the target sequence of the gRNA, and wherein the one or more donor DNA template sequences recombine with the one or more target nucleic acid sequences to insert, delete, and/or substitute one or more bases of the sequence of the one or more target nucleic acid sequences to induce one or more sequence modifications at the one or more target loci within the genome.

In some embodiments, the msr and msd regions of the retron transcript form a secondary structure, wherein the formation of the secondary structure is facilitated by base pairing between the first and second inverted repeat sequences, and wherein the secondary structure is recognized by the RT for the initiation of reverse transcription. In some embodiments, the RNA-guided nuclease is Cas9 or Cpf1. In some embodiments, the one or more donor DNA sequences comprise two homology arms, wherein each homology arm has at least about 70% to about 99% similarity to a portion of the mammalian genetic locus on either side of the gRNA target sequence. In some embodiments, the isolated mammalian host cell is a human cell. In some embodiments, about ten or more target loci are modified. In some embodiments, the host cell comprises a population of host cells. In some embodiments, the method further comprises introducing a single-strand annealing protein (SSAP) into the host cell.

In another aspect, the present disclosure provides a method for screening one or more genetic loci of interest in a genome of a mammalian host cell, the method comprising: (a) modifying one or more target nucleic acids of interest at one or more target loci within the genome of the host cell according to any of the herein-described methods; (b) incubating the modified host cell under conditions sufficient to elicit a phenotype that is controlled by the one or more genetic loci of interest; (c) identifying the resulting phenotype of the modified host cell; and (d) determining that the identified phenotype was the result of the modifications made to the one or more target nucleic acids of interest at the one or more target loci of interest.

In some embodiments, at least 1,000 to 1,000,000 genetic loci of interest are screened simultaneously. In some embodiments, the phenotype is identified using a reporter. In some embodiments, the reporter is selected from the group consisting of a fluorescent tagged protein, an antibody, a chemical stain, a chemical indicator, and a combination thereof. In some embodiments, the reporter responds to the concentration of a metabolic product, a protein product, a synthesized drug of interest, a cellular phenotype of interest, or a combination thereof.

In another aspect, the present disclosure provides a mammalian host cell that has been transformed by any of the herein-described vectors.

In another aspect, the present disclosure provides a pharmaceutical composition comprising: (a) any of the herein-described guide RNA-retron cassettes, vectors, gRNA-msr-msd-donor RNA molecules, or a combination thereof, and (b) a pharmaceutically acceptable carrier.

In another aspect, the present disclosure provides a method for preventing or treating a genetic disease in a subject, the method comprising administering to the subject an effective amount of any of the herein-disclosed pharmaceutical compositions to correct a mutation in a target gene associated with the genetic disease.

In some embodiments, the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, ocular diseases and disorders, and a combination thereof.

In another aspect, the present disclosure provides a kit for modifying one or more target nucleic acids of interest at one or more target loci within a genome of a host cell, the kit comprising one or a plurality of any of the herein-described vectors.

In some embodiments, the kit further comprises a mammalian host cell. In some embodiments, the kit further comprises one or more reagents for transforming the host cell with the one or plurality of vectors, one or more reagents for inducing expression of one or more cassettes within the one or plurality of vectors, or a combination thereof. In some embodiments, the kit further comprises instructions for transforming the host cell, inducing expression of the one or more cassettes within the one or plurality of vectors, or a combination thereof.

In another aspect, the present disclosure provides a kit for modifying one or more target nucleic acids of interest at one or more target loci within a genome of a host cell, the kit comprising one or a plurality of any of the herein-described gRNA-msr-msd-donor RNA molecules.

In some embodiments, the kit further comprises a mammalian host cell. In some embodiments, the kit further comprises one or more reagents for introducing the one or plurality of gRNA-msr-msd-donor RNA molecules into the mammalian host cell. In some embodiments, the kit further comprises an RNA-guided nuclease-RT fusion protein or a plasmid for expressing an RNA-guided nuclease-RT fusion protein. In some embodiments, the kit further comprises instructions for introducing the one or plurality of gRNA-msr-msd-donor RNA molecules into the mammalian host cell, inducing expression of the RNA-guided nuclease-RT fusion protein, or a combination thereof. In some embodiments, the RNA-guided nuclease is saCas9, spCas9, or Cpf1.

### BRIEF DESCRIPTION OF THE DRAWINGS

FIGS. 1A-1C. Retrons produce msDNA in human cells. (FIG. 1A) Schematic of our strategy to deploy retrons to generate gRNA-msDNA hybrids as intracellular donors. (FIG. 1B) Schematic showing construct design for the qPCR assay: guide RNA and msr-msd expression was driven by the human U6 promoter; human codon-optimized RT and SpCas9 expression was driven by the CBh promoter; donor templates were inserted into the replaceable regions of msd. T2A, P2A: self-cleaving peptides. (FIG. 1C) Relative abundance of msDNA produced by different retrons. DNA amplified from the same volume of OBZ206 ssDNA at 1×10⁻⁷ ng/μL was set as one-fold to calculate the relative abundance. Data presented as mean±s.d. (n=2 experiments). NTC: non-transfected control. Cas9 only: cells transfected with the plasmid without gRNA or any retron, gRNA+Cas9: cells transfected with the plasmid expressing both gRNA and Cas9, w/o msr/msd: cells transfected with plasmid that co-express gRNA, Cas9 and RT, but no msr/msd was inserted. Other labels in the X axis indicate cells transfected with plasmids that carry different retron sequences.

FIGS. 2A-2D. Retrons enable HDR in K562 and 293T BFP reporter cells. (FIG. 2A) Schematic showing the principle of BFP reporter cell line, adapted from Richardson et al. (22) (FIG. 3a). (FIG. 2B) Schematic of plasmid design. (FIG. 2C) The percentage of BFP− cells, indicating a lower bound on the SpCas9 cutting efficiency in BFP-to-GFP conversion K562 reporter cells after being transfected with different DNA components. (FIG. 2D) The percentage of GFP+ cells, indicating the HDR editing efficiency in BFP-to-GFP conversion K562 reporter cells after being transfected with different DNA components. In FIG. 2C and FIG. 2D, data are presented as the mean±SD of three biological replicates. Mean values are shown above bars. *p<0.05; **p<0.01; ***p<0.001; ****p<0.0001; p≥0.05, n.s. Statistical analysis was carried out using one-way ANOVA. NTC, non-transfected control; Cas9 only, cells transfected with the plasmid without gRNA or any retron, gBFP+Cas9, cells transfected with the plasmid expressing both gBFP and Cas9; gBFP-An+Cas9, cells transfected with the plasmid expressing gBFP, An, and Cas9; gBFP+Cas9-An ssDNA, cells co-transfected with the plasmid expressing gBFP and Cas9, and the synthesized An ssDNA; gBFP+Cas9-At ssDNA, cells co-transfected with the plasmid expressing gBFP and Cas9, and the synthesized At ssDNA; gBFP-An+Cas9-Sa163, cells co-transfected with the plasmid expressing gBFP. An, and Cas9-Sa163; gBFP-An+Cas9-Ec86, cells co-transfected with the plasmid expressing gBFP, An, and Cas9-Ec86. “An” and “At” are ssDNA donor sequences of BFP-to-GFP conversion.

FIGS. 3A-3B. (FIG. 3A) A typical amplification curve generated in qPCR assay. During the linear-log phase, PCR products of the target gene approximately double in each cycle. Amplification stops at the plateau phase. (FIG. 3B) The synthesized DNA oligo OBZ206 was used as a “ladder” to indicate the qPCR linear-log phase in this test. Data presented as mean±s.d. (n=3).

FIGS. 4A-4E. All FACS plots related to FIGS. 2A-2D. (FIG. 4A) Gating strategy to detect the HDR rate as shown in FIG. 2D. From left to right, all cells were first gated for size by forward scatter area (FSC-A) and side scatter area (SSC-A); single cells were further selected by side scatter area (SSC-A) and side scatter height (SSC-H); then cells transfected with retron-CRISPR plasmids were determined by YL2-A (mCherry-A); subsequently, BFP to GFP conversion rates were measured by VL-A(BFP-A) and BL-A(GFP-A). Since the mCherry gene was carried by a plasmid, for non-transfected control (NTC) cells, the BFP to GFP conversion rate was measured among all single cell populations. (FIG. 4B) Schematic of the three pairs of target strand (At, Dt, Ht) and non-target strand (An, Dn, Hn) donor templates tested, adapted from Richardson et al. (FIG. 3c) (22). (FIG. 4C) The graph summarizes the HDR percentages achieved by Ec86 and Sa163 among variable donor templates. (FIG. 4D) All FACS plots tested in K562 BFP reporter cells. (FIG. 4E) All FACS plots tested in HEK293T BFP reporter cells.

### DETAILED DESCRIPTION

### I. Introduction

The present disclosure provides methods and compositions for the retron-mediated delivery of homologous donor templates to mammalian cells, including human cells. The present methods and compositions provide numerous advantages over previous methods and compositions for effecting genomic editing in mammalian cells. For example, existing gene correction systems require extracellular DNA donor delivery for HDR, whereas the present methods enable intracellular donor generation in human cells, which is easy-to-use and cost-effective. Because extracellular DNA donors are not renewable in cells after delivery, current gene therapies cannot attain life-long gene editing in pediatric patients since vectors are diluted due to high cell turnover as young patients develop (C. J. Stephens et al, eds., (2019)). The present disclosure provides methods and compositions using retrons to express desired DNA donors in human cells, delivering a promising solution for treating young patients. In addition, the herein-described CRISPEY gRNA-retron design allows the gRNA and msDNA to be covalently linked, making the donor template immediately available for HDR repair at Cas9-induced DSBs. Further, the present methods (e.g., human CRISPEY, or hCRISPEY) provide advantages over base editor gene correction tools in that base editors can only correct single nucleotides, whereas the hCRISPEY can introduce multiple nucleotide alterations simultaneously, which is suitable for diseases caused by multiple mutations or large structural variants. e.g., cancer and Alzheimer's disease. In addition, the present methods provide greater specificity than prime editor gene correction tools: in the CRISPEY platform, the reverse transcriptase (RT) only targets the msr-msd from the same retron as its RNA template, providing specificity that could, e.g., help avoid off-target reverse transcription.

The present methods and compositions comprise various improvements over other CRISPEY systems. e.g., yeast CRISPEY, that enable high-throughput, precision genome editing in mammalian (e.g., human) cells. For example, in some embodiments, the reverse transcriptase (RT) is fused to Cas9 (or other RNA-guided nuclease) to increase the local concentration of donor DNA in the proximity of the Cas9 cut site. This allows HDR to occur even at low donor concentrations, while simultaneously preventing large-scale toxicity that would impact the cell's normal functions. In addition, in some embodiments, in the mammalian (e.g., human) CRISPEY platform the msr-msd-donor is attached at the 3′ end of the gRNA, to protect gRNA from RNase degradation. Furthermore, in some embodiments, the human CRIPSEY platform is effective in msDNA generation and precise gene editing without the inclusion of a self-cleaving RNA sequence (known as a ribozyme), making the human CRISPEY platform easier to use than the previous yeast version. Moreover, in some embodiments, the gRNA-retron cassette in the hCRISPEY system comprises an msd locus comprising one or more sequence modifications to avoid pre-mature RNA polymerase III termination.

### II. General

The practice of the present methods and compositions employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, Molecular Cloning: A Laboratory Manual, 2nd edition (1989), Current Protocols in Molecular Biology (F. M. Ausubel, et al, eds., (1987)), the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies. A Laboratory Manual, and Animal Cell Culture (R. I. Freshney, ed. (1987)).

For nucleic acids, sizes are given in either kilobases (kb), base pairs (bp), or nucleotides (nt). Sizes of single-stranded DNA and/or RNA can be given in nucleotides. These are estimates derived from agarose or acrylamide gel electrophoresis, from sequenced nucleic acids, or from published DNA sequences. For proteins, sizes are given in kilodaltons (kDa) or amino acid residue numbers. Protein sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage and Caruthers, Tetrahedron Lett. 22:1859-1862 (1981), using an automated synthesizer, as described in Van Devanter et. al., Nucleic Acids Res. 12:6159-6168 (1984). Purification of oligonucleotides is performed using any art-recognized strategy, e.g., native acrylamide gel electrophoresis or anion-exchange high performance liquid chromatography (HPLC) as described in Pearson and Reanier, J. Chrom. 255: 137-149 (1983).

### III. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosure. For the purposes of the present disclosure, the following terms are defined.

The terms “a.” “an.” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The term “about” in relation to a reference numerical value can include a range of values plus or minus 10% from that value. For example, the amount “about 10” includes amounts from 9 to 11, including the reference numbers of 9, 10, and 11. The term “about” in relation to a reference numerical value can also include a range of values plus or minus 10%, 9%, 8%, 7%, 6%, 5%, 4%, 3%, 2%, or 1% from that value.

As used herein, unless otherwise specified, the terms “5′” and “3′” denote the positions of elements or features relative to the overall arrangement of the retron-guide RNA cassettes, vectors, or retron donor DNA-guide molecules of the present disclosure in which they are included. Positions are not, unless otherwise specified, referred to in the context of the orientation of a particular element or features. For example, the msr and msd loci are shown in opposite orientations. However, the msr locus is said to be 5′ of the msd locus. Furthermore, the 3′ end of the msr locus is said to be overlapping with the 5′ end of the msd locus. Unless otherwise specified, the term “upstream” refers to a position that is 5′ of a point of reference. Conversely, the term “downstream” refers to a position that is 3′ of a point of reference.

The term “genome editing” or “genomic editing” or “genetic editing” refers to a type of genetic engineering in which DNA is inserted, replaced, or removed from a target DNA (e.g., the genome of a cell) using one or more nucleases and/or nickases. The nucleases create specific double-strand breaks (DSBs) at desired locations in the genome, and harness the cell's endogenous mechanisms to repair the induced break by homology-directed repair (HDR) (e.g., homologous recombination) or by non-homologous end joining (NHEJ). The nickases create specific single-strand breaks at desired locations in the genome. In one non-limiting example, two nickases can be used to create two single-strand breaks on opposite strands of a target DNA, thereby generating a blunt or a sticky end. Any suitable DNA nuclease can be introduced into a cell to induce genome editing of a target DNA sequence.

The term “DNA nuclease” refers to an enzyme capable of cleaving the phosphodiester bonds between the nucleotide subunits of DNA, and may be an endonuclease or an exonuclease. According to the present disclosure, the DNA nuclease may be an engineered (e.g., programmable or targetable) DNA nuclease which can be used to induce genome editing of a target DNA sequence. Any suitable DNA nuclease can be used including, but not limited to, CRISPR-associated protein (Cas) nucleases, other endo- or exo-nucleases, variants thereof, fragments thereof, and combinations thereof. DNA nucleases that are guided to specific sequences within the genome by specific RNA molecules. e.g., Cas nucleases guided to target sequences by gRNAs, are referred to as “RNA-guided nucleases”.

The term “double-strand break” or “double-strand cut” refers to the severing or cleavage of both strands of the DNA double helix. The DSB may result in cleavage of both stands at the same position leading to “blunt ends” or staggered cleavage resulting in a region of single-stranded DNA at the end of each DNA fragment, or “sticky ends”. A DSB may arise from the action of one or more DNA nucleases.

The term “non-homologous end joining” or “NHEJ” refers to a pathway that repairs double-strand DNA breaks in which the break ends are directly ligated without the need for a homologous template.

The term “homology-directed repair” or “HDR” refers to a mechanism in cells to accurately and precisely repair double-strand DNA breaks using a homologous template to guide repair. The most common form of HDR is homologous recombination (HR), a type of genetic recombination in which nucleotide sequences are exchanged between two similar or identical molecules of DNA.

The term “nucleic acid,” “nucleotide,” or “polynucleotide” refers to deoxyribonucleic acids (DNA), ribonucleic acids (RNA) and polymers thereof in either single-, double- or multi-stranded form. The term includes, but is not limited to, single-, double- or multi-stranded DNA or RNA, genomic DNA, cDNA, DNA-RNA hybrids, or a polymer comprising purine and/or pyrimidine bases or other natural, chemically modified, biochemically modified, non-natural, synthetic or derivatized nucleotide bases. In some embodiments, a nucleic acid can comprise a mixture of DNA, RNA and analogs thereof. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “single nucleotide polymorphism” or “SNP” refers to a change of a single nucleotide within a polynucleotide, including within an allele. This can include the replacement of one nucleotide by another, as well as the deletion or insertion of a single nucleotide. Most typically. SNPs are biallelic markers although tri- and tetra-allelic markers can also exist. By way of non-limiting example, a nucleic acid molecule comprising SNP A\C may include a C or A at the polymorphic position.

The term “gene” means the segment of DNA involved in producing a polypeptide chain. The DNA segment may include regions preceding and following the coding region (leader and trailer) involved in the transcription/translation of the gene product and the regulation of the transcription/translation, as well as intervening sequences (introns) between individual coding segments (exons).

The term “cassette” refers to a combination of genetic sequence elements that may be introduced as a single element and may function together to achieve a desired result. A cassette typically comprises polynucleotides in combinations that are not found in nature.

The term “operably linked” refers to two or more genetic elements, such as a polynucleotide coding sequence and a promoter, placed in relative positions that permit the proper biological functioning of the elements, such as the promoter directing transcription of the coding sequence.

The term “inducible promoter” refers to a promoter that responds to environmental factors and/or external stimuli that can be artificially controlled in order to modify the expression of, or the level of expression of, a polynucleotide sequence or refers to a combination of elements, for example an exogenous promoter and an additional element such as a trans-activator operably linked to a separate promoter. An inducible promoter may respond to abiotic factors such as oxygen levels or to chemical or biological molecules. In some embodiments, the chemical or biological molecules may be molecules not naturally present in humans.

The terms “vector” and “expression vector” refer to a nucleic acid construct, generated recombinantly or synthetically, with a series of specified nucleic acid elements that permit transcription of a particular polynucleotide sequence in a host cell. An expression vector may be part of a plasmid, viral genome, or nucleic acid fragment. Typically, an expression vector includes a polynucleotide to be transcribed, operably linked to a promoter. The term “promoter” is used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. Other elements that may be present in an expression vector include those that enhance transcription (e.g., enhancers) and terminate transcription (e.g., terminators).

“Recombinant” refers to a genetically modified polynucleotide, polypeptide, cell, tissue, or organism. For example, a recombinant polynucleotide (or a copy or complement of a recombinant polynucleotide) is one that has been manipulated using well known methods. A recombinant expression cassette comprising a promoter operably linked to a second polynucleotide (e.g., a coding sequence) can include a promoter that is heterologous to the second polynucleotide as the result of human manipulation (e.g., by methods described in Sambrook et al., Molecular Cloning—A Laboratory Manual, Cold Spring Harbor Laboratory, Cold Spring Harbor, New York, (1989) or Current Protocols in Molecular Biology Volumes 1-3, John Wiley & Sons, Inc. (1994-1998)). A recombinant expression cassette (or expression vector) typically comprises polynucleotides in combinations that are not found in nature. For instance, human manipulated restriction sites or plasmid vector sequences can flank or separate the promoter from other sequences. A recombinant protein is one that is expressed from a recombinant polynucleotide, and recombinant cells, tissues, and organisms are those that comprise recombinant sequences (polynucleotide and/or polypeptide).

As used herein, the term “heterologous” refers to biological material that is introduced, inserted, or incorporated into a recipient (e.g., host) organism that originates from another organism. Typically, the heterologous material that is introduced into the recipient organism (e.g., a host cell) is not normally found in that organism. Heterologous material can include, but is not limited to, nucleic acids, amino acids, peptides, proteins, and structural elements such as genes, promoters, and cassettes. A host cell can be, but is not limited to, a bacterium, a yeast cell, a mammalian cell, or a plant cell. The introduction of heterologous material into a host cell or organism can result, in some instances, in the expression of additional heterologous material in or by the host cell or organism. As a non-limiting example, the transformation of a human host cell with an expression vector that contains DNA sequences encoding a bacterial protein may result in the expression of the bacterial protein by the human cell. The incorporation of heterologous material may be permanent or transient. Also, the expression of heterologous material may be permanent or transient.

The terms “reporter” and “selectable marker” can be used interchangeably and refer to a gene product that permits a cell expressing that gene product to be identified and/or isolated from a mixed population of cells. Such isolation might be achieved through the selective killing of cells not expressing the selectable marker, which may be, as a non-limiting example, an antibiotic resistance gene. Alternatively, the selectable marker may permit identification and/or subsequent isolation of cells expressing the marker as a result of the expression of a fluorescent protein such as GFP or the expression of a cell surface marker which permits isolation of cells by fluorescence-activated cell sorting (FACS), magnetic-activated cell sorting (MACS), or analogous methods. Suitable cell surface markers include CD8, CD19, and truncated CD19. Preferably, cell surface markers used for isolating desired cells are non-signaling molecules, such as subunit or truncated forms of CD8, CD19, or CD20. Suitable markers and techniques are known in the art.

The terms “culture,” “culturing,” “grow.” “growing,” “maintain,” “maintaining.” “expand.” “expanding.” etc., when referring to cell culture itself or the process of culturing, can be used interchangeably to mean that a cell (e.g., human cell) is maintained outside its normal environment under controlled conditions, e.g., under conditions suitable for survival. Cultured cells are allowed to survive, and culturing can result in cell growth, stasis, differentiation or division. The term does not imply that all cells in the culture survive, grow, or divide, as some may naturally die or senesce. Cells are typically cultured in media, which can be changed during the course of the culture.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo, cultured in vitro, or modified ex vivo, are also encompassed.

As used herein, the term “administering” includes oral administration, topical contact, administration as a suppository, intravenous, intraperitoneal, intramuscular, intralesional, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “treating” refers to an approach for obtaining beneficial or desired results including, but not limited to, a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “sufficient amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific amount may vary depending on one or more of: the particular agent chosen, the host cell type, the location of the host cell in the subject, the dosing regimen to be followed, whether it is administered in combination with other compounds, timing of administration, and the physical delivery system in which it is carried.

The term “pharmaceutically acceptable carrier” refers to a substance that aids the administration of an active agent to a cell, an organism, or a subject. “Pharmaceutically acceptable carrier” refers to a carrier or excipient that can be included in the present compositions and that causes no significant adverse toxicological effect on the patient. Non-limiting examples of pharmaceutically acceptable carrier include water, NaCl, normal saline solutions, lactated Ringer's, normal sucrose, normal glucose, cell culture media, and the like. One of skill in the art will recognize that other pharmaceutical carriers are useful in the present methods and compositions.

The term “cellular localization tag” refers to an amino acid sequence, also known as a “protein localization signal.” that targets a protein for localization to a specific cellular or subcellular region, compartment, or organelle (e.g., nuclear localization sequence, Golgi retention signal). Cellular localization tags are typically located at either the N-terminal or C-terminal end of a protein. A database of protein localization signals (LocSigDB) is maintained online by the University of Nebraska Medical Center (genome.unmc.edu/LocSigDB). For more information regarding cellular localization tags, see, e.g., Negi, et al. Database (Oxford). 2015: bav003 (2015); incorporated herein by reference in its entirety for all purposes.

The term “synthetic response element” refers to a recombinant DNA sequence that is recognized by a transcription factor and facilitates gene regulation by various regulatory agents. A synthetic response element can be located within a gene promoter and/or enhancer region.

The term “ribozyme” refers to an RNA molecule that is capable of catalyzing a biochemical reaction. In some instances, ribozymes function in protein synthesis, catalyzing the linking of amino acids in the ribosome. In other instances, ribozymes participate in various other RNA processing functions, such as splicing, viral replication, and tRNA biosynthesis. In some instances, ribozymes can be self-cleaving. Non-limiting examples of ribozymes include the HDV ribozyme, the Lariat capping ribozyme (formally called GIR1 branching ribozyme), the glmS ribozyme, group I and group II self-splicing introns, the hairpin ribozyme, the hammerhead ribozyme, various rRNA molecules, RNase P, the twister ribozyme, the VS ribozyme, the pistol ribozyme, and the hatchet ribozyme. For more information regarding ribozymes, see. e.g., Doherty, et al. Ann. Rev. Biophys. Biomol. Struct. 30: 457-475 (2001); incorporated herein by reference in its entirety for all purposes.

“Percent similarity,” in the context of polynucleotide or peptide sequences, is determined by comparing two optimally aligned sequences over a comparison window, wherein the portion of the sequence (e.g., an msr locus sequence) in the comparison window may comprise additions or deletions (i.e., gaps) as compared to the reference sequence which does not comprise additions or deletions, for optimal alignment of the two sequences. The percentage is calculated by determining the number of positions at which the identical nucleotide or amino acid occurs in both sequences to yield the number of matched positions, dividing the number of matched positions by the total number of positions in the window of comparison and multiplying the result by 100 to yield the percentage of similarity (e.g., sequence similarity).

When a polynucleotide or peptide has at least about 70% similarity (e.g., sequence similarity), preferably at least about 85%, 86%, 87%, 88%, 89%, 90%, 91%, 92%, 93%, 94%, 95%, 96%, 97%, 98%, 99%, or 100% similarity, to a reference sequence, when compared and aligned for maximum correspondence over a comparison window, or designated region as measured using one of the following sequence comparison algorithms or by manual alignment and visual inspection, such sequences are then said to be “substantially similar.” With regard to polynucleotide sequences, this definition also refers to the complement of a test sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence similarities for the test sequences relative to the reference sequence, based on the program parameters. For sequence comparison of nucleic acids and proteins, the BLAST and BLAST 2.0 algorithms and the default parameters discussed below are used.

Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection (see, e.g., Current Protocols in Molecular Biology (Ausubel et al., eds. 1995 supplement)).

Additional examples of algorithms that are suitable for determining percent sequence similarity are the BLAST and BLAST 2.0 algorithms, which are described in Altschul et al., (1990). J. Mol. Biol. 215: 403-410 and Altschul et al. (1977) Nucleic Acids Res. 25: 3389-3402, respectively. Software for performing BLAST analyses is publicly available at the National Center for Biotechnology Information website, nebi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see. e.g., Henikoff and Henikoff, Proc. Natl. Acad. Sci. USA 89:10915 (1989)).

The BLAST algorithm also performs a statistical analysis of the similarity between two sequences (see. e.g., Karlin and Altschul, Proc. Nat'l. Acad. Sci. USA, 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

### IV. Detailed Description of the Embodiments

The present disclosure provides compositions and methods for high-throughput genome editing and screening in mammalian cells. The disclosure provides methods comprising the use of guide RNA-retron cassettes, Cas9 (or other nuclease) —reverse transcriptase (RT) fusion proteins and cassettes encoding said fusion proteins, vectors comprising said cassettes, and retron donor DNA template-guide molecules as described herein to modify nucleic acids of interest at mammalian target loci of interest, and to screen mammalian genetic loci of interest, in the genomes of mammalian host cells. The present disclosure also provides compositions and methods for preventing or treating genetic diseases in mammals by enhancing precise genome editing to correct a mutation in target genes associated with the diseases. Kits for genome editing and screening are also provided. The present methods and compositions are suitable for use with any mammalian cell type and at any gene locus that is amenable to nuclease-mediated genome editing technology.

### A. The CRISPR-Retron System

In a first aspect, the present disclosure provides a guide RNA (gRNA)-retron cassette. In some embodiments, the guide RNA (gRNA)-retron cassette comprises: (a) a gRNA coding region, wherein the target sequence of the gRNA is within a mammalian genetic locus; and (b) a retron region comprising: (i) an msr locus; (ii) a first inverted repeat sequence; (iii) an msd locus; (iv) a donor DNA template region located within the msd locus, wherein the donor DNA template is homologous to one or more sequences within the mammalian genetic locus; and (v) a second inverted repeat sequence. In particular embodiments, the first inverted repeat sequence is located within the 5′ end of the msr locus and/or the second inverted repeat sequence is located 3′ of the msd locus.

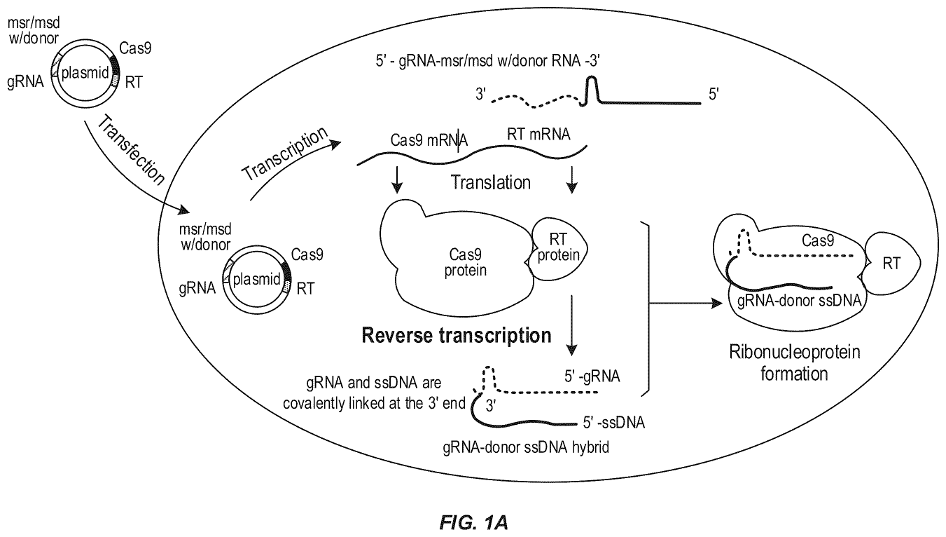

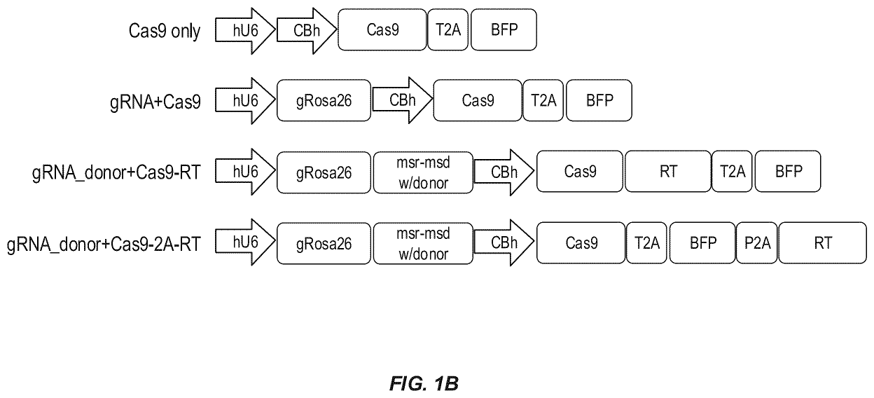

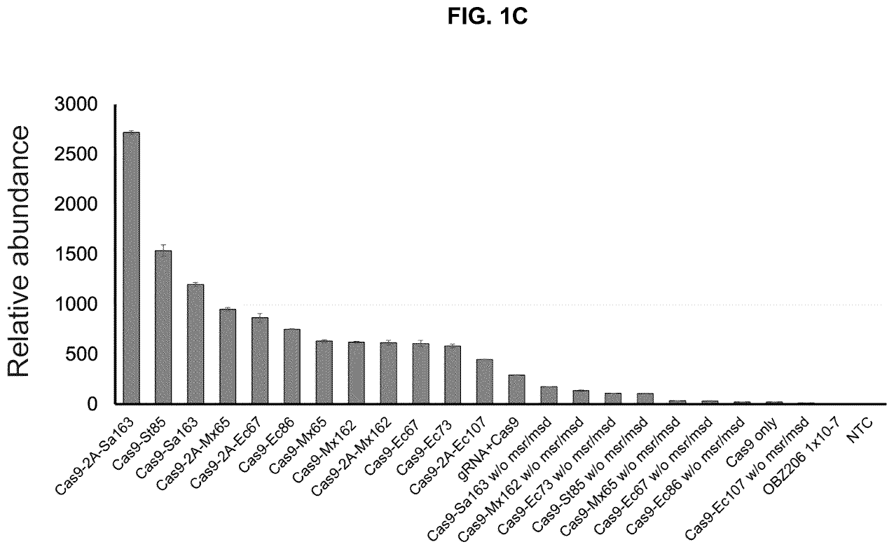

In such embodiments, transcription of the gRNA-retron cassette produces a gRNA-msr-msd-donor RNA molecule, e.g., an RNA molecule comprising (a) a guide RNA (gRNA), wherein the target sequence of the gRNA is within a mammalian genetic locus; and (b) a retron transcript comprising: (i) an msr region; (ii) a first inverted repeat sequence; (iii) an msd region; (iv) a donor DNA template coding region located within the msd region, wherein the donor DNA template is homologous to the mammalian genetic locus; and (v) a second inverted repeat sequence. In particular embodiments, the gRNA is 5′ of the retron transcript within the RNA molecule (see, e.g., FIG. 1A). In particular embodiments, upon reverse transcription of the retron transcript to produce a single-stranded DNA (ssDNA) donor DNA template, the resulting gRNA and donor template ssDNA molecules are covalently linked, e.g., at their 3″ ends (see. e.g., FIG. 1A), i.e., the donor DNA sequence is physically coupled to the gRNA, by virtue of the ssDNA being physically coupled to the gRNA. In some embodiments, transcription of the gRNA-msr-msd-donor RNA molecule is driven by an RNA polymerase III promoter, e.g., U6 (SEQ ID NO:55).

In another aspect, the present disclosure provides an expression cassette comprising a polynucleotide encoding reverse transcriptase fused to Cas9 or another RNA-guided nuclease (e.g., Cpf1). In particular embodiments, the coding sequence for the RT and Cas9 fusion protein within the cassette is driven by an RNA polymerase II promoter, e.g., CBh (SEQ ID NO:56). In some embodiments, upon transcription the RT coding sequence is 3′ of the Cas9 coding sequence within the transcript. In some embodiments, the reverse transcriptase (RT) coding sequence may further comprise a nuclear localization sequence (NLS). e.g., a nucleoplasmin NLS (SEQ ID NO:57), and the Cas9 coding sequence may further comprise an NLS such as the simian virus 40 NLS (SV40 NLS) (SEQ ID NO:58). In some instances, the NLS is located at the 3′ end of the RT coding sequence and SV40 NLS is located at the 5′ end of the Cas9 coding sequence.

In particular embodiments, the gRNA-retron cassette and the Cas9-RT cassette are present within a single, multicistronic vector (see. e.g., FIGS. 1A and 1B).

In some embodiments, the gRNA-retron cassette and/or the RT-Cas9 fusion cassette is at least about 5.000 nucleotides in length. In other embodiments, the gRNA-retron cassette and/or the RT-Cas9 fusion cassette is between about 1,000 and 5,000 (i.e., about 1,000, 1,100, 1,200, 1.300, 1,400, 1,500, 1,600, 1,700, 1,800, 1,900, 2.000, 2, 100, 2.200, 2,300, 2,400, 2,500, 2,600, 2,700, 2,800, 2,900, 3,000, 3, 100, 3,200, 3,300, 3,400, 3,500, 3,600, 3,700, 3,800, 3,900, 4.000, 4.100, 4.200, 4.300, 4.400, 4.500, 4.600, 4.700, 4.800, 4.900, or 5,000) nucleotides in length. In some other embodiments, the gRNA-retron cassette and/or the RT-Cas9 fusion cassette is between about 300 and 1,000 (i.e., about 300, 350, 400, 450, 500, 550, 600, 650, 700, 750, 800, 850, 900, 950, or 1,000) nucleotides in length. In particular embodiments, the gRNA-retron cassette and/or the RT-Cas9 fusion cassette is between about 200 and 300 (i.e., about 200, 210, 220, 230, 240, 250, 260, 270, 280, 290, or 300) nucleotides in length. In other embodiments, the gRNA-retron cassette and/or the RT-Cas9 fusion cassette is between about 30 and 200 (i.e., about 30, 40, 50, 60, 70, 80, 90, 100, 110, 120, 130, 140, 150, 160, 170, 180, 190, or 200) nucleotides in length. In some embodiments, the gRNA-retron cassette and/or the RT-Cas9 fusion cassette is about 200 (i.e., between about 100 and 300, 150 and 250, 175 and 225, or 190 and 210) nucleotides in length.

In other embodiments, the cassette further comprises one or more sequences having homology to a vector cloning site. These vector homology sequences can be about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, or more nucleotides in length. In some instances, the vector homology sequences are about 20 nucleotides in length. In other instances, the vector homology sequence are about 15 nucleotides in length. In yet other instances, the vector homology sequences are about 25 nucleotides in length.

In particular embodiments, the promoter within the chimeric gRNA-msr-msd cassette, which can be referred to as a chimeric molecule, and/or the RT-Cas9 fusion cassette is inducible. In some instances, the promoter is an RNA polymerase II promoter. In other instances, the promoter is an RNA polymerase III promoter. In particular instances, a combination of promoters is used. In some other embodiments, the vector further comprises a terminator sequence. Vectors of the present disclosure can include commercially available recombinant expression vectors and fragments and variants thereof. Examples of suitable promoters and recombinant expression vectors are described herein and will also be known to one of skill in the art.

In some embodiments, the vector contains a reporter unit that includes, e.g., a nucleotide sequence encoding a reporter polypeptide (e.g., a detectable polypeptide, fluorescent polypeptide, or a selectable marker (e.g., mCherry)).

The size of the vector will depend on the size of the individual components within the vector. e.g., gRNA-retron cassette, Cas9-RT coding sequence, reporter unit, and so on. In some embodiments, the vector is less than about 1,000 (i.e., less than about 1,000, 950, 900, 850, 800, 750, 700, 650, 600, 550, or 500) nucleotides in length. In other embodiments, the vector is between about 1,000 and about 20,000 (i.e., about 1,000, 1.500, 2,000, 2,500, 3,000, 3,500, 4,000, 4,500, 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16.500, 17,000, 17,500, 18,000, 18.500, 19,000, 19,500, or 20,000) nucleotides in length. In particular embodiments, the vector is more than about 20,000 nucleotides in length.

### 1. Retrons

Retrons represent a class of retroelement, first discovered in gram-negative bacteria such as Myxococcus xanthus (e.g., retrons Mx65 and Mx162). Stigmatella aurantiaca (e.g., retron Sa163), and Escherichia coli (e.g., retrons Ec48, Ec67, Ec73, Ec78, Ec83, Ec86, and Ec107). Retrons are also found in Salmonella typhimurium (e.g., retron St85), Salmonella enteritidis, Vibrio cholerae (e.g., retron Ve95). Vibrio parahaemolyticus (e.g., retron Vp96), Klebsiella pneumoniae. Proteus mirabilis, Xanthomonas campestris. Rhizobium sp., Bradyrhizobium sp., Ralstonia metallidurans, Nannocystis exedens (e.g., retron Ne144), Geobacter sulfurreducens, Trichodesmium erythraeum, Nostoc punctiforme, Nostoc sp., Staphylococcus aureus, Fusobacterium nucleanim, and Flexibacter elegans. In one aspect, the present disclosure provides for guide RNA-retron cassettes that comprise a retron. In some embodiments, the retron is derived from the E. coli retron Ec86 (e.g., Uniprot: P23070).

Retrons mediate the synthesis in host cells of multicopy single-stranded DNA (msDNA) molecules, which result from the reverse transcription of a retron transcript and typically include an RNA component (msr) and a DNA component (msd). The native msDNA molecules reportedly exist as single-stranded RNA-DNA hybrids, characterized by a structure which comprises a single-stranded DNA branching out of an internal guanosine residue of a single-stranded RNA molecule at a 2′,5′-phosphodiester linkage. In some embodiments, at least some of the RNA content of the msDNA molecule is degraded. In some instances, the RNA content is degraded by RNase H.

The msd region of a retron transcript typically codes for the DNA component of msDNA, and the msr region of a retron transcript typically codes for the RNA component of msDNA. In some retrons, the msr and msd loci have overlapping ends (see, e.g., J. Biol. Chem., 268(4): 2684-92 (1993)), and may be oriented opposite one another with a promoter located upstream of the msr locus which transcribes through the msr and msd loci. One of skill in the art will appreciate that the sequence of the msd locus will vary, depending on the particular donor DNA sequence that is located within the msd locus.

The msd and msr regions of retron transcripts generally contain first and second inverted repeat sequences, which together make up a stable stem structure. The combined msr-msd region of the retron transcript serves not only as a template for reverse transcription but, by virtue of its secondary structure, also serves as a primer (i.e., self-priming) for msDNA synthesis by a reverse transcriptase. In some embodiments of the herein-disclosed guide RNA-retron cassettes, the first inverted repeat sequence is located within the 5′ end of the msr locus. In other embodiments, the second inverted repeat sequence is located 3′ of the msd locus. In some embodiments of the herein-disclosed retron transcript, the first inverted repeat sequence is located within the 5′ end of the msr region. In other embodiments, the second inverted repeat sequence is located 3′ of the msd region.

Any number of RTs may be used in alternative embodiments of the present disclosure, including prokaryotic and eukaryotic RTs. If desired, the nucleotide sequence of a native RT may be modified, for example using known codon optimization techniques, so that expression within the desired mammalian host is optimized. By codon optimization it is meant the selection of appropriate DNA nucleotides for the synthesis of oligonucleotide building blocks, and their subsequent enzymatic assembly, of a structural gene or fragment thereof in order to approach codon usage within the host.

The RT may be targeted to the nucleus so that efficient utilization of the RNA template may take place. An example of such a RT includes any known RT, either prokaryotic or eukaryotic, fused to a nuclear localization sequence or signal (NLS). In some embodiments of vectors of the present disclosure, the vector further comprises an NLS. In particular embodiments, the NLS is located 3′ of the RT coding sequence. Any suitable NLS may also be used, providing that the NLS assists in localizing the RT within the nucleus. The use of an RT in the absence of an NLS may also be used if the RT is present within the nuclear compartment at a level that synthesizes a product from the RNA template.

For more information regarding retrons, see, e.g., U.S. Pat. No. 8,932,860 and Lampson, et al. Cytogenet. Res. 110:491-499 (2005); both incorporated herein by reference in their entirety for all purposes.

2. Guide RNA (gRNA) Molecules

The guide RNA (gRNA)-retron cassettes and gRNA-msr-msd-donor RNA molecules of the present disclosure comprise guide RNA (gRNA) coding regions and gRNA molecules, respectively. The gRNAs for use in the CRISPR-retron system as disclosed herein typically include a crRNA sequence that is complementary to a target nucleic acid sequence and may include a scaffold sequence (e.g., SEQ ID NO:59) comprising a crRNA repeat sequence (e.g., SEQ ID NO:60) and a tracrRNA sequence (e.g., SEQ ID NO:61) that interacts with a Cas nuclease (e.g., Cas9) or a variant or fragment thereof, depending on the particular nuclease being used.

The gRNA can comprise any nucleic acid sequence having sufficient complementarity with a target polynucleotide sequence (e.g., target genomic DNA sequence) to hybridize with the target sequence and direct sequence-specific binding of a nuclease to the target sequence. The gRNA may recognize a protospacer adjacent motif (PAM) sequence that may be near or adjacent to the target DNA sequence. The target DNA site may lie immediately 5′ of a PAM sequence, which is specific to the bacterial species of the Cas9 used. For instance, the PAM sequence of Streptococcus pyogenes-derived Cas9 is NGG; the PAM sequence of Neisseria meningitidis-derived Cas9 is NNNNGATT; the PAM sequence of Streptococcus thermophilus-derived Cas9 is NNAGAA; and the PAM sequence of Treponema denticola-derived Cas9 is NAAAAC. In some embodiments, the PAM sequence can be 5′-NGG, wherein N is any nucleotide; 5′-NRG, wherein N is any nucleotide and R is a purine; or 5′-NNGRR, wherein N is any nucleotide and R is a purine. For the S. pyogenes system, the selected target DNA sequence should immediately precede (i.e., be located 5′ of) a 5′NGG PAM, wherein N is any nucleotide, such that the guide sequence of the DNA-targeting RNA (e.g., gRNA) base pairs with the opposite strand to mediate cleavage at about 3 base pairs upstream of the PAM sequence.

In other instances, the target DNA site may lie immediately 3′ of a PAM sequence, e.g., when the Cpf1 endonuclease is used. In some embodiments, the PAM sequence is 5′-TTIN, where N is any nucleotide. When using the Cpf1 endonuclease, the target DNA sequence (i.e., the genomic DNA sequence having complementarity for the gRNA) will typically follow (i.e., be located 3′ of) the PAM sequence. Two CPI-family nucleases, AsCpf1 (from Acidaminococcus) and LbCpf1 (from Lachnospiraceae) are known to function in human cells. Both AsCpf1 and LbCpf1 cut 19 bp after the PAM sequence on the targeted strand and 23 bp after the PAM sequence on the opposite strand of the DNA molecule.

In some embodiments, the degree of complementarity between a guide sequence of the gRNA (i.e., crRNA sequence) and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT. Novoalign (Novocraft Technologies, ELAND (Illumina, San Diego, Calif.). SOAP (available at soap.genomics.org.cn), and Maq (available at maq.sourceforge.net). In some embodiments, a crRNA sequence is about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some instances, a crRNA sequence is about 20 nucleotides in length. In other instances, a crRNA sequence is about 15 nucleotides in length. In other instances, a crRNA sequence is about 25 nucleotides in length.

The nucleotide sequence of a modified gRNA can be selected using any of the web-based software described above. Considerations for selecting a DNA-targeting RNA include the PAM sequence for the nuclease (e.g., Cas9 or Cpf1) to be used, and strategies for minimizing off-target modifications. Tools, such as the CRISPR Design Tool, can provide sequences for preparing the gRNA, for assessing target modification efficiency, and/or assessing cleavage at off-target sites.

In some embodiments, the length of the gRNA molecule is about 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, or more nucleotides in length. In some instances, the length of the gRNA is about 100 nucleotides in length. In other instances, the gRNA is about 90 nucleotides in length. In other instances, the gRNA is about 110 nucleotides in length.

### 3. Donor DNA Sequences

In one aspect, the present disclosure provides guide RNA (gRNA)-retron cassettes comprising an msd with an embedded donor DNA sequence. In another aspect, the present disclosure provides gRNA-msr-msd-donor RNA molecules comprising guide RNA-msr-msd transcripts that comprise donor DNA sequence coding regions, the transcripts subsequently being reverse transcribed to yield msDNA that comprises a donor DNA sequence. The donor DNA sequence or sequences participate in homology-directed repair (HDR) of genetic loci of interest following cleavage of genomic DNA at the genetic locus or loci of interest (i.e., after a nuclease has been directed to cut at a specific genetic locus of interest, targeted by binding of gRNA to a target sequence).

In some embodiments, the recombinant donor repair template (i.e., donor DNA sequence) comprises two homology arms that are homologous to portions of the sequence of the genetic locus of interest at either side of a Cas nuclease (e.g., Cas9 or Cpf1 nuclease) cleavage site. The homology arms may be the same length or may have different lengths. In some instances, each homology arm has at least about 70 to about 99 percent similarity (i.e., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent similarity) to a portion of the sequence of the genetic locus of interest at either side of a nuclease (e.g., Cas nuclease) cleavage site. In other embodiments, the recombinant donor repair template comprises or further comprises a reporter unit that includes a nucleotide sequence encoding a reporter polypeptide (e.g., a detectable polypeptide, fluorescent polypeptide, or a selectable marker). If present, the two homology arms can flank the reporter cassette and are homologous to portions of the genetic locus of interest at either side of the Cas nuclease cleavage site. The reporter unit can further comprise a sequence encoding a self-cleavage peptide, one or more nuclear localization signals, and/or a fluorescent polypeptide (e.g., superfolder GFP (sfGFP)). Other suitable reporters are described herein.

In some embodiments, the donor DNA sequence is at least about 500 to 10,000 (i.e., at least about 500, 600, 700, 800, 900, 1,000, 1,100, 1,200, 1,300, 1,400, 1,500, 1,600, 1,700, 1,800, 1.900, 2,000, 2,500, 3,000, 3,500, 4,000, 4,500, 5.000, 5,500, 6.000, 6,500, 7,000, 7,500, 8,000, 8,500, 9,000, 9.500, or 10.000) nucleotides in length. In some embodiments, the donor DNA sequence is between about 600 and 1,000 (i.e., about 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1.000) nucleotides in length. In some embodiments, the donor DNA sequence is between about 100 and 500 (i.e., about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or 500) nucleotides in length. In some embodiments, the donor DNA sequence is less than about 100 (i.e., less than about 100, 95, 90, 85, 80, 75, 70, 65, 60, 55, 50, 45, 40, 35, 30, 25, 20, 15, 10, or 5) nucleotides in length.

### B. CRISPR/Cas System

The CRISPR/Cas system of genome modification includes a Cas nuclease (e.g., Cas9 or Cpf1 nuclease) or a variant or fragment or combination thereof and a DNA-targeting RNA (e.g., guide RNA (gRNA)). The gRNA may contain a guide sequence that targets the Cas nuclease to the target genomic DNA and a scaffold sequence that interacts with the Cas nuclease (e.g., tracrRNA). The system may optionally include a donor repair template. In other instances, a fragment of a Cas nuclease or a variant thereof with desired properties (e.g., capable of generating single- or double-strand breaks and/or modulating gene expression) can be used. The donor repair template can include a nucleotide sequence encoding a reporter polypeptide such as a fluorescent protein or an antibiotic resistance marker, and homology arms that are homologous to the target DNA and flank the site of gene modification.

The CRISPR (Clustered Regularly Interspaced Short Palindromic Repeats)/Cas (CRISPR-associated protein) nuclease system is an engineered nuclease system based on a bacterial system that can be used for genome engineering. It is based on part of the adaptive immune response of many bacteria and archaea. When a virus or plasmid invades a bacterium, segments of the invader's DNA are converted into CRISPR RNAs (crRNA) by the “immune” response. The crRNA then associates, through a region of partial complementarity, with another type of RNA called traerRNA to guide the Cas (e.g., Cas9) nuclease to a region homologous to the crRNA in the target DNA called a “protospacer.” The Cas (e.g., Cas9) nuclease cleaves the DNA to generate blunt ends at the double-strand break at sites specified by a 20-nucleotide guide sequence contained within the crRNA transcript. The Cas (e.g., Cas9) nuclease may require both the crRNA and the traerRNA for site-specific DNA recognition and cleavage. This system has now been engineered such that the crRNA and traerRNA, if needed, can be combined into one molecule (the “single guide RNA” or “sgRNA”), and the crRNA equivalent portion of the guide RNA can be engineered to guide the Cas (e.g., Cas9) nuclease to target any desired sequence (see, e.g., Jinek et al. (2012) Science, 337:816-821; Jinek et al. (2013) eLife, 2:e00471; Segal (2013) eLife, 2:e00563). Thus, the CRISPR/Cas system can be engineered to create a double-strand break at a desired target in a genome of a cell, and harness the cell's endogenous mechanisms to repair the induced break by homology-directed repair (HDR) or nonhomologous end-joining (NHEJ).

The Cas nuclease can direct cleavage of one or both strands at a location in a target DNA sequence. For example, the Cas nuclease can be a nickase having one or more inactivated catalytic domains that cleaves a single strand of a target DNA sequence.

Non-limiting examples of Cas nucleases include Cas1, Cas1B, Cas2, Cas3, Cas4, Cas5, Cas6, Cas7, Cas8. Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, homologs thereof, variants thereof, fragments thereof, mutants thereof, derivatives thereof, and combinations thereof. There are three main types of Cas nucleases (type I, type II, and type III), and 10 subtypes including 5 type I, 3 type II, and 2 type III proteins (see, e.g., Hochstrasser and Doudna, Trends Biochem Sci, 2015:40(1):58-66). Type II Cas nucleases include Cas1, Cas2, Csn2, Cas9, and Cpf1. These Cas nucleases are known to those skilled in the art. For example, the amino acid sequence of the Streptococcus pyogenes wild-type Cas9 polypeptide is set forth, e.g., in NBCI Ref. Seq. No. NP_269215, and the amino acid sequence of Streptococcus thermophilus wild-type Cas9 polypeptide is set forth, e.g., in NBCI Ref. Seq. No. WP_011681470. Furthermore, the amino acid sequence of Acidaminococcus sp. BV3L6 is set forth, e.g., in NBCI Ref. Seq. No. WP_021736722.1. Some CRISPR-related endonucleases that are useful in the present methods and compositions are disclosed, e.g., in U.S. Application Publication Nos. 2014/0068797, 2014/0302563, and 2014/0356959.

Cas nucleases, e.g., Cas9 polypeptides, can be derived from a variety of bacterial species including, but not limited to, Veillonella atypical, Fusobacterium nucleatum, Filifactor alocis, Solobacterium moorei, Coprococcus catus, Treponema denticola, Peptoniphilus duerdenii, Catenibacterium mitsuokai, muitains, Streptococcus Listeria innocua, Staphylococcus pseudintermedius, Acidaminococcus intestine, Olsenella uli, Oenococcus kitaharae. Bifidobacterium bifidum, Lactobacillus rhamnosus, Lactobacillus gasseri, Finegoldia magna, Mycoplasma mobile, Mycoplasma gallisepticum, Mycoplasma ovipneumoniae, Mycoplasma canis, Mycoplasma synoviae, Eubacterium rectale, Streptococcus thermophilus, Eubacterium dolichum, Lactobacillus coryniformis subsp. Torquens, Ilyobacter polytropus, Ruminococcus albus, Akkermansia muciniphila, Acidothermus cellulolyticus, Bifidobacterium longum, Bifidobacterium dentium, Corynebacterium diphtheria, Elusimicrobium minutum, Nitratifractor salsuginis, Sphaerochaeta globus, Fibrobacter succinogenes subsp. Succinogenes, Bacteroides fragilis, Capnocytophaga ochracea, Rhodopseudomonas palustris, Prevotella micans, Prevotella ruminicola, Flavobacterium columnare, Aminomonas paucivorans, Rhodospirillum rubrum, Candidatus Puniceispirillum marinum, Verminephrobacter eiseniae, Ralstonia syzygii, Dinoroseobacter shibae. Azospirillum, Nitrobacter hamburgensis. Bradyrhizobium, Wolinella succinogenes. Campylobacter jejuni subsp. Jejuni, Helicobacter mustelae, Bacillus cereu, Acidovorax ebreus, Clostridium perfringens, Parvibaculum lavamentivorans, Roseburia intestinalis, Neisseria meningitidis, Pasteurella multocida subsp. Multocida, Sutterella wadsworthensis, proteobacterium, Legionella pneumophila, Parasutterella excrementihominis, Wolinella succinogenes, and Francisella novicida.

“Cpf1” refers to an RNA-guided double-stranded DNA-binding nuclease protein that is a type II Cas nuclease. Wild-type Cpf1 contains a RuvC-like endonuclease domain similar to the RuvC domain of Cas9, but does not have an HNH endonuclease domain and the N-terminal region of Cpf1 does not have the alpha-helix recognition lobe possessed by Cas9. The wild-type protein requires a single RNA molecule, as no tracrRNA is necessary. Wild-type Cpf1 creates staggered-end cuts and utilizes a T-rich protospacer-adjacent motif (PAM) that is 5′ of the guide RNA targeting sequence. Cpf1 enzymes have been isolated, for example, from Acidaminococcus and Lachnospiraceae.

“Cas9” refers to an RNA-guided double-stranded DNA-binding nuclease protein or nickase protein that is a type II Cas nuclease. Wild-type Cas9 nuclease has two functional domains, e.g., RuvC and HNH, that cut different DNA strands. The wild-type enzyme requires two RNA molecules (e.g., a crRNA and a tracrRNA), or alternatively, a single fusion molecule (e.g., a gRNA comprising a crRNA and a tracrRNA). Wild-type Cas9 utilizes a G-rich protospacer-adjacent motif (PAM) that is 3′ of the guide RNA targeting sequence and creates double-strand cuts having blunt ends. Cas9 can induce double-strand breaks in genomic DNA (target DNA) when both functional domains are active. The Cas9 enzyme can comprise one or more catalytic domains of a Cas9 protein derived from bacteria belonging to the group consisting of Corynebacter, Sutterella, Legionella, Treponema, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, and Campylobacter. In some embodiments, the two catalytic domains are derived from different bacteria species.

Useful variants of the Cas9 nuclease can include a single inactive catalytic domain, such as a RuvC⁻ or HNH⁻ enzyme or a nickase. A Cas9 nickase has only one active functional domain and can cut only one strand of the target DNA, thereby creating a single-strand break or nick. A double-strand break can be introduced using a Cas9 nickase if at least two DNA-targeting RNAs that target opposite DNA strands are used. A double-nicked induced double-strand break can be repaired by NHEJ or HDR (Ran et al., 2013, Cell, 154:1380-1389). This gene editing strategy favors HDR and decreases the frequency of insertion/deletion (“indel”) mutations at off-target DNA sites. Non-limiting examples of Cas9 nucleases or nickases are described in, for example, U.S. Pat. Nos. 8,895,308; 8,889,418; and 8,865,406 and U.S. Application Publication Nos. 2014/0356959, 2014/0273226 and 2014/0186919. The Cas9 nuclease or nickase can be codon-optimized for the host cell or host organism.

For genome editing methods, the Cas nuclease can be a Cas9 fusion protein such as a polypeptide comprising the catalytic domain of a restriction enzyme (e.g., FokI) linked to dCas9. The FokI-dCas9 fusion protein (fCas9) can use two guide RNAs to bind to a single strand of target DNA to generate a double-strand break.

In some embodiments, a nucleotide sequence encoding the Cas nuclease is present in a recombinant expression vector. In certain instances, the recombinant expression vector is a viral construct, e.g., a recombinant adeno-associated virus construct, a recombinant adenoviral construct, a recombinant lentiviral construct, etc. For example, viral vectors can be based on vaccinia virus, poliovirus, adenovirus, adeno-associated virus, SV40, herpes simplex virus, human immunodeficiency virus, and the like. A retroviral vector can be based on Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, mammary tumor virus, and the like. Useful expression vectors are known to those of skill in the art, and many are commercially available. The following vectors are provided by way of example for eukaryotic host cells: pXT1, pSG5, pSVK3, pBPV, pMSG, and pSVLSV40. However, any other vector may be used if it is compatible with the host cell. For example, useful expression vectors containing a nucleotide sequence encoding a Cas9 enzyme are commercially available from, e.g., Addgene, Life Technologies, Sigma-Aldrich, and Origene.

Depending on the mammalian host cell and expression system used, any of a number of transcription and translation control elements, including promoter, transcription enhancers, transcription terminators, and the like, may be used in the expression vector. Useful promoters can be derived from viruses, or any organism, e.g., eukaryotic organisms. Promoters may also be inducible (i.e., capable of responding to environmental factors and/or external stimuli that can be artificially controlled). Suitable promoters include, but are not limited to: RNA polymerase II (Pol II) promoters, RNA polymerase III (Pol III) promoters, the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), an enhanced U6 promoter, a human H1 promoter (H1), etc. Suitable terminators include, but are not limited to SNR52 and RPR terminator sequences, which can be used with transcripts created under the control of an RNA polymerase III (Pol III) promoter. Additionally, various primer binding sites may be incorporated into a vector to facilitate vector cloning, sequencing, genotyping, and the like. Other suitable promoter, enhancer, terminator, and primer binding sequences will readily be known to one of skill in the art.

In some embodiments, the gRNA-retron cassette comprises an msd locus comprising one or more (e.g., two, three, four, five, or more) sequence modifications (e.g., single nucleotide substitutions) to accommodate Pol III transcription (e.g., to avoid or prevent pre-mature Pol Ill termination). In certain embodiments, the msd locus comprises a modification of a “TTTT” sequence to a “TTTc” or “TTTa” sequence in the stem region. In particular embodiments, a “TTTT” sequence in the stem region of an Ec86, Ec107, or St85 msd sequence is modified. Exemplary Ec86, Ec107, and St85 msd sequences comprising such modifications are set forth in SEQ ID NOS: 6, 11, and 18, respectively. In certain other embodiments, the msd locus further comprises a modification of a corresponding sequence in the opposite strand of the stem region for maintaining secondary structure. As non-limiting examples, the msd locus may further comprise a modification of a corresponding “GGAAA” or “GAAAA” sequence to a “GGgAA” or “GgAAA” sequence, respectively. In particular embodiments, a corresponding “GGAAA” sequence in the stem region of an Ec86 msd sequence or a corresponding “GAAAA” sequence in the stem region of an St85 msd sequence is modified. Exemplary Ec86 and St85 msd sequences comprising such modifications are set forth in SEQ ID NOS: 7 and 19, respectively. In yet other embodiments, the msd locus further comprises a modification of a “TTTTTT” sequence to a “TTTcTT” sequence downstream (i.e., 3″) of the stem region. In particular embodiments, a “TTITIT” sequence downstream of the stem region of an Ec86 msd sequence is modified. An exemplary Ec86 msd sequence comprising such a modification is set forth in SEQ ID NO. 8. In some embodiments, the expression vector comprises a Pol III promoter (e.g., U6) and a gRNA-retron cassette comprising an msd locus modified as described herein to avoid or prevent pre-mature Pol III termination.

The gRNA-retron cassettes and vectors provided by the present disclosure comprising a modified msd locus to eliminate premature Pol III termination are particularly advantageous for one or more of the following reasons: (1) the full msr-msd sequence can be transcribed in order for msDNA to be produced; (2) no leader sequence is required at the 5′ of the gRNA, which is critical for gRNA cutting efficiency; (3) generation of higher transcript number for higher efficiency in editing relative to Pol II; (4) additional structured non-coding RNA (other than gRNA) that are optimized for Pol III transcription can be attached to the retron RNA; (5) Pol III transcription is nuclear and preferred for Cas9 and RT function, compared to Pol II with cap and polyA tailing mechanisms that promote RNA export to cytoplasm; (6) the Pol III promoter can be shorter to comply with vector constraints, to generate more compact vectors for delivery; and/or (7) the Pol Ill promoter is more widely activated across tissue- and cell-types, allowing one vector design for different tissue targets and indications.

The compositions and methods provided by the gRNA-retron cassettes and vectors comprising a modified msd locus to eliminate premature Pol III termination are useful for any number of applications. In some embodiments, the modified msd locus enhances gRNA-retron expression by Pol III, enabling other Pol III-optimized RNA elements to be attached to the gRNA-retron and expressed as a chimeric molecule. As non-limiting examples, gRNA-retrons attached to Pol III-optimized riboswitches or aptamers can be conditionally targeted for activation or deactivation by small molecule drugs to allow local or temporal activation of gene editing (e.g., tunable gene editing) in vivo; gRNA-retrons attached to Pol III-optimized fluorescent RNAs can be visualized in vivo and in vitro to indicate delivery, localization, and abundance of gRNA-retron molecules in vivo to allow assaying of gene editing activity by gRNA-retron in vivo; and gRNA-retrons attached to Pol III-optimized natural or synthetic RNA regulatory elements such as RNA-binding protein binding sites can be conditionally activated or deactivated based on tissue or cell state to allow tissue- or cell-type specific gene editing in vivo.

In some embodiments, other agents for promoting or improving the efficiency of CRISPR/Cas mediated genomic editing can be introduced into the mammalian host cell, e.g., as a protein or a polynucleotide encoding a protein. In particular embodiments, a single-strand annealing protein (SSAP), or a polynucleotide encoding an SSAP, is introduced.

C. Methods for Introducing Nucleic Acids into Host Cells

Methods for introducing polypeptides and nucleic acids into a host cell are known in the art, and any known method can be used to introduce a nuclease or a nucleic acid (e.g., a nucleotide sequence encoding the nuclease or reverse transcriptase, a DNA-targeting RNA (e.g., a guide RNA), a donor repair template for homology-directed repair (HDR), etc.) into a cell. Non-limiting examples of suitable methods include electroporation, viral infection, transfection, lipofection, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

In some embodiments, the components of the CRISPR-retron system can be introduced into a cell using a delivery system. In certain instances, the delivery system comprises a nanoparticle, a microparticle (e.g., a polymer micropolymer), a liposome, a micelle, a virosome, a viral particle, a nucleic acid complex, a transfection agent, an electroporation agent (e.g., using a NEON transfection system), a nucleofection agent, a lipofection agent, and/or a buffer system that includes a nuclease component (as a polypeptide or encoded by an expression construct), a reverse transcriptase component, and one or more nucleic acid components such as a DNA-targeting RNA (e.g., a guide RNA) and/or a donor repair template. For instance, the components can be mixed with a lipofection agent such that they are encapsulated or packaged into cationic submicron oil-in-water emulsions. Alternatively, the components can be delivered without a delivery system, e.g., as an aqueous solution.

Methods of preparing liposomes and encapsulating polypeptides and nucleic acids in liposomes are described in, e.g., Methods and Protocols, Volume 1: Pharmaceutical Nanocarriers: Methods and Protocols. (ed. Weissig). Humana Press, 2009 and Heyes et al. (2005) J Controlled Release 107:276-87. Methods of preparing microparticles and encapsulating polypeptides and nucleic acids are described in, e.g., Functional Polymer Colloids and Microparticles volume 4 (Microspheres, microcapsules & liposomes). (eds. Arshady & Guyot). Citus Books, 2002 and Microparticulate Systems for the Delivery of Proteins and Vaccines. (eds. Cohen & Bernstein). CRC Press, 1996.

### D. Host Cells

In a particular aspect, the present disclosure provides host cells that have been transformed by vectors of the present disclosure. The compositions and methods of the present disclosure can be used for genome editing of any mammalian host cell of interest. The mammalian host cell can be a cell from, e.g., a human, from a healthy human, from a human patient, from a cancer patient, etc. In some cases, the host cell treated by the method disclosed herein can be transplanted to a subject (e.g., patient). For instance, the host cell can be derived from the subject to be treated (e.g., patient).

Any type of cell may be of interest, such as a stem cell, e.g., embryonic stem cell, induced pluripotent stem cell, adult stem cell. e.g., mesenchymal stem cell, neural stem cell, hematopoietic stem cell, organ stem cell, a progenitor cell, a somatic cell, e.g., fibroblast, hepatocyte, heart cell, liver cell, pancreatic cell, muscle cell, skin cell, blood cell, neural cell, immune cell, and any other cell of the body. e.g., human body. The cells can be primary cells or primary cell cultures derived from a subject, e.g., an animal subject or a human subject, and allowed to grow in vitro for a limited number of passages. In some embodiments, the cells are disease cells or derived from a subject with a disease. For instance, the cells can be cancer or tumor cells. The cells can also be immortalized cells (e.g., cell lines), for instance, from a cancer cell line.

Cells can be harvested from a subject by any standard method. For instance, cells from tissues, such as skin, muscle, bone marrow, spleen, liver, kidney, pancreas, lung, intestine, stomach, etc., can be harvested by a tissue biopsy or a fine needle aspirate. Blood cells and/or immune cells can be isolated from whole blood, plasma or serum. In some cases, suitable primary cells include peripheral blood mononuclear cells (PBMC), peripheral blood lymphocytes (PBL), and other blood cell subsets such as, but not limited to, T cell, a natural killer cell, a monocyte, a natural killer T cell, a monocyte-precursor cell, a hematopoietic stem cell or a non-pluripotent stem cell. In some cases, the cell can be any immune cells including any T-cell such as tumor infiltrating cells (TILs), such as CD3+ T-cells, CD4+ T-cells, CD8+ T-cells, or any other type of T-cell. The T cell can also include memory T cells, memory stem T cells, or effector T cells. The T cells can also be skewed towards particular populations and phenotypes. For example, the T cells can be skewed to phenotypically comprise, CD45RO(−), CCR7(+), CD45RA(+), CD62L(+), CD27(+), CD28(+) and/or IL-7Rα(+). Suitable cells can be selected that comprise one of more markers selected from a list comprising: CD45RO(−), CCR7(+), CD45RA(+), CD62L(+), CD27(+), CD28(+) and/or IL-7Rα(+). Induced pluripotent stem cells can be generated from differentiated cells according to standard protocols described in, for example, U.S. Pat. Nos. 7,682,828, 8,058,065, 8,530,238, 8,871,504, 8,900,871 and 8,791,248, the disclosures are herein incorporated by reference in their entirety for all purposes.

In some embodiments, the host cell is in vitro. In other embodiments, the host cell is ex vivo. In yet other embodiments, the host cell is in vivo.

### E. Methods for Genome Editing and Screening, and Assessing the Efficiency and Precision Thereof

In another aspect, the present disclosure provides a method for modifying one or more target nucleic acids of interest at one or more target loci within a genome of a host cell. In some embodiments, the method comprises:
(a) transforming the host cell with a vector of the present disclosure; and
(b) culturing the host cell or transformed progeny of the host cell under conditions sufficient for expressing from the vector a gRNA-msr-msd donor molecule comprising an msr-msd-donor transcript and a guide RNA (gRNA) molecule,
wherein the msr-msd-donor transcript self-primes reverse transcription by a reverse transcriptase (RT) expressed by the host cell or the transformed progeny of the host cell,
wherein at least a portion of the retron transcript is reverse transcribed to produce a multicopy single-stranded DNA (msDNA) molecule having one or more donor DNA sequences, wherein the one or more donor DNA sequences are homologous to the one or more target loci and comprise sequence modifications compared to the one or more target nucleic acids,
wherein the one or more target loci are cut by a nuclease expressed by the host cell or the transformed progeny of the host cell, wherein the site of nuclease cutting is specified by the gRNA, and
wherein the one or more donor DNA sequences recombine with the one or more target nucleic acid sequences to insert, delete, and/or substitute one or more bases of the sequence of the one or more target nucleic acid sequences to induce one or more sequence modifications at the one or more target loci within the genome.

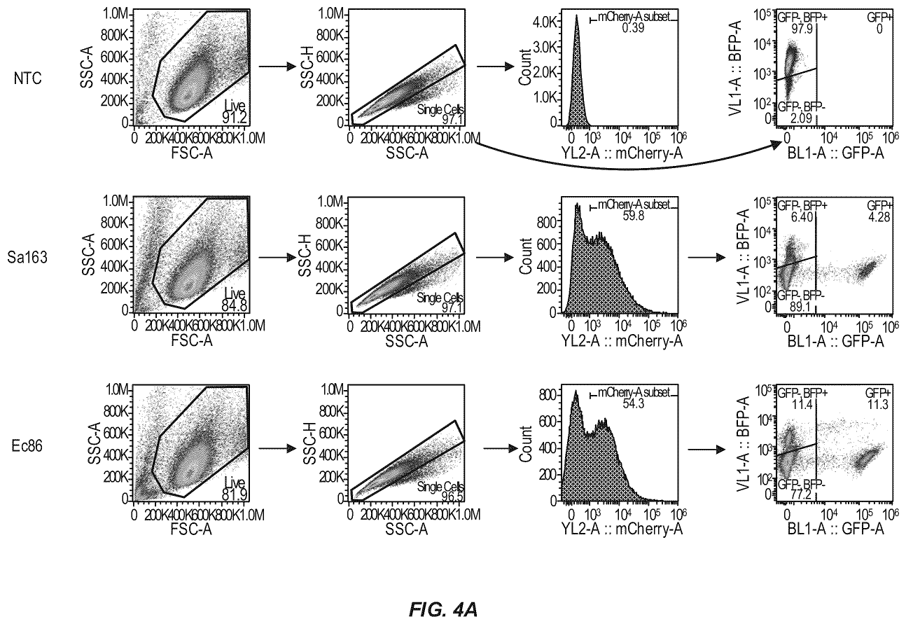

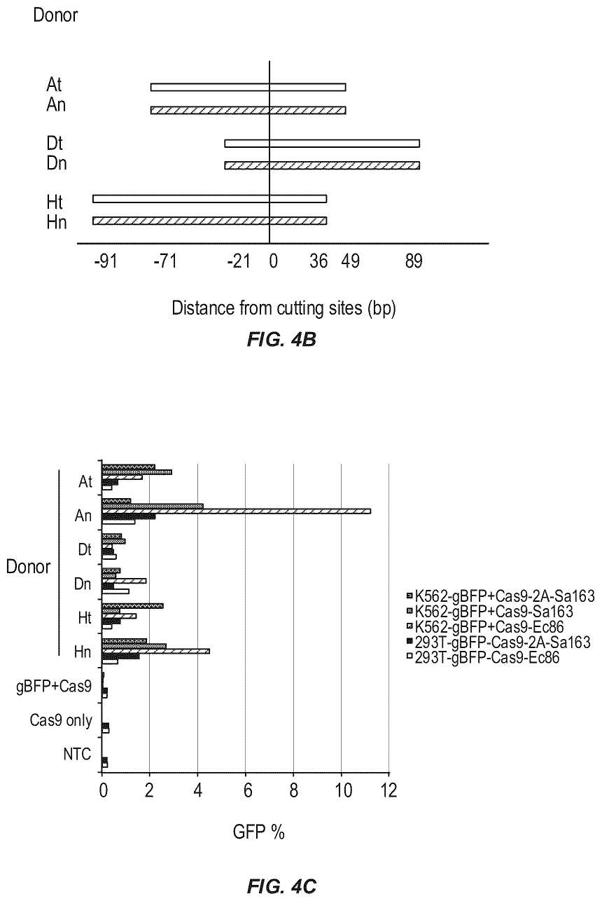

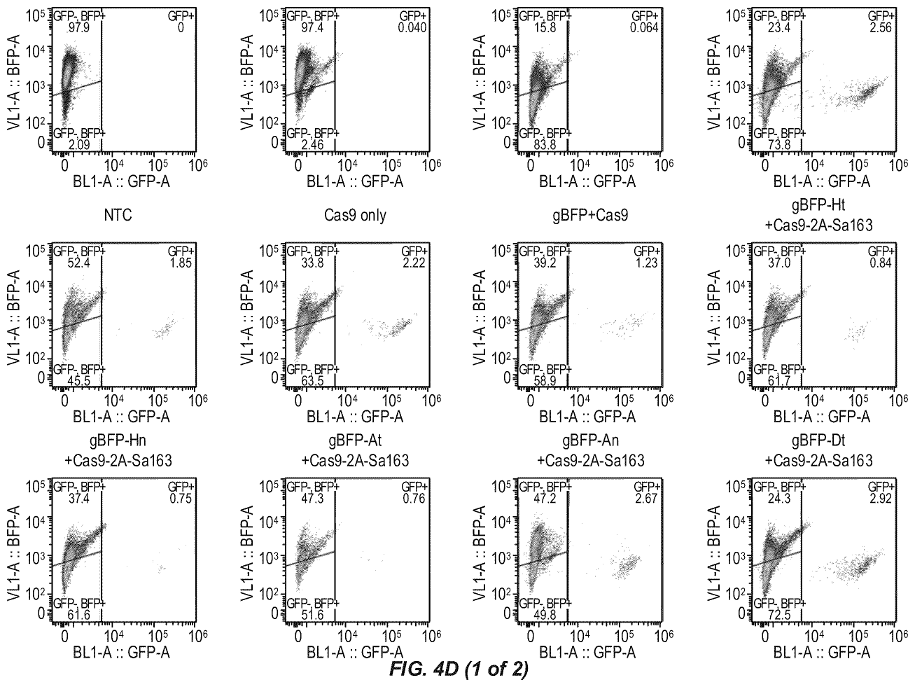

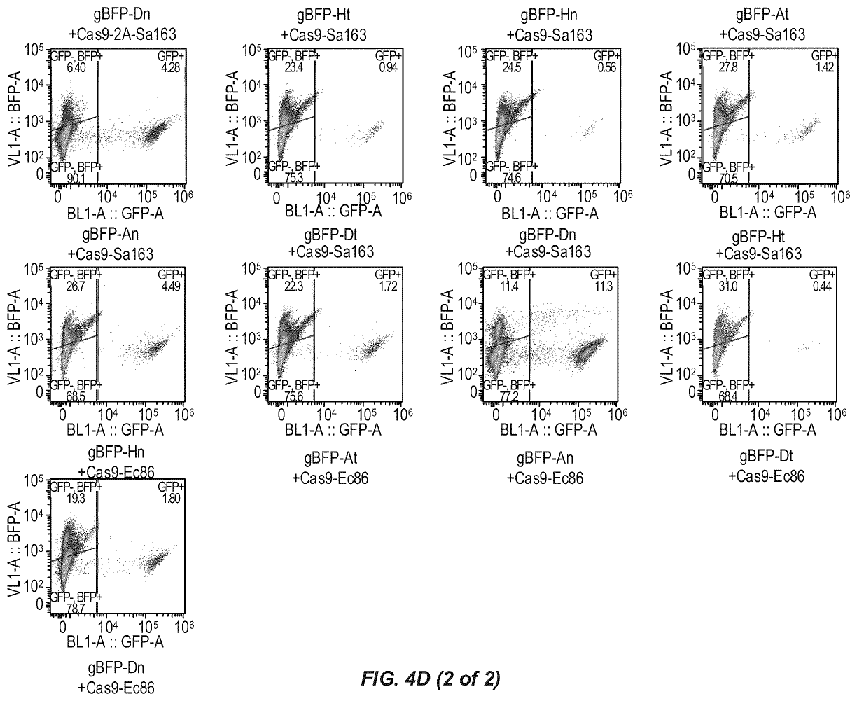

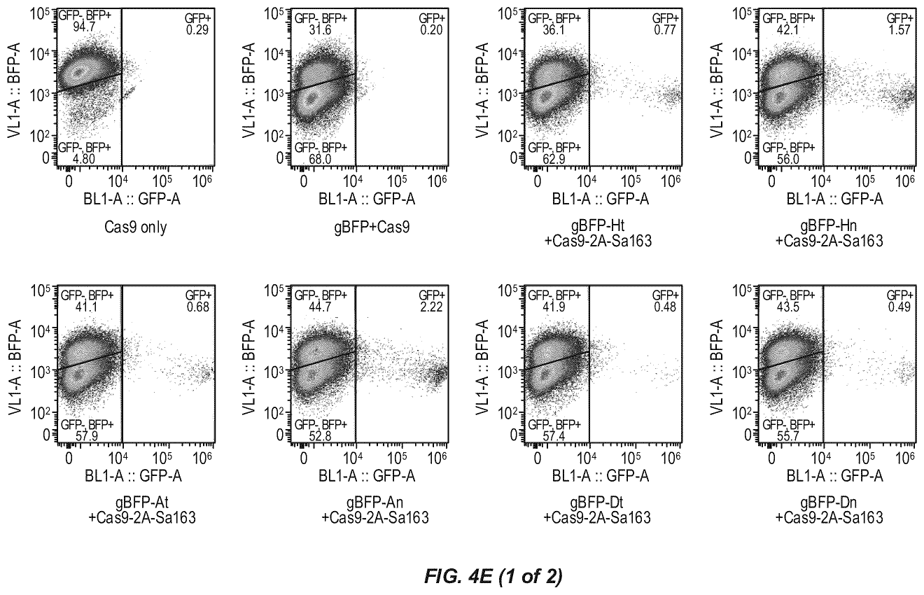

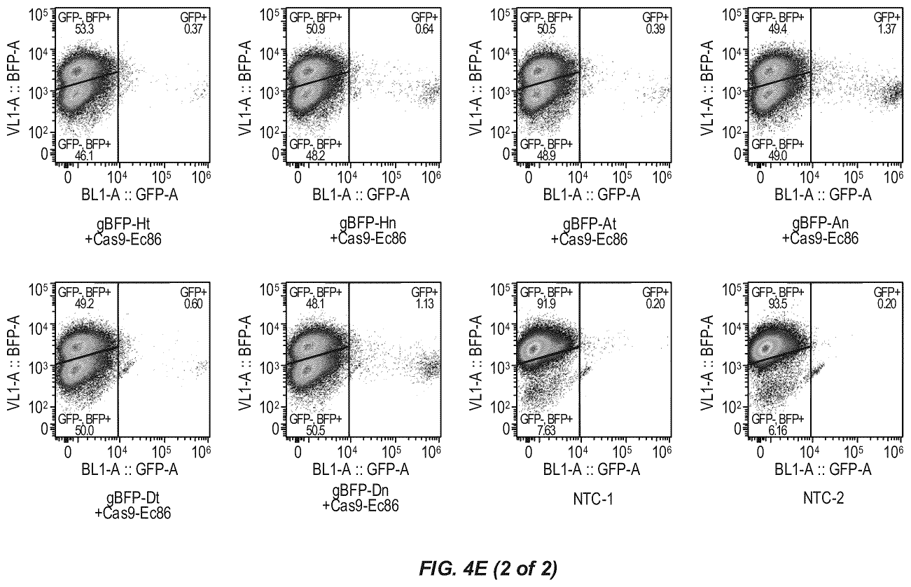

In some embodiments, the RT is present within an RT-nuclease (e.g., Cas9) fusion protein, e.g., encoded by a cassette within the vector or integrated into the genome of the host cell. In some embodiments, RT and Cas9 coding sequences are present within a non-fusion, bicistronic Cas9-RT protein cassette separated by a self-cleaving peptide (e.g., P2A, T2A, F2A, E2A). e.g., as shown in human cells in FIGS. 4C-4E.

In yet another aspect, the present disclosure provides a method for screening one or more genetic loci of interest in a genome of a host cell, the method comprising:
(a) modifying one or more target nucleic acids of interest at one or more target loci within the genome of the host cell according to a method of the present disclosure;
(b) incubating the modified host cell under conditions sufficient to elicit a phenotype that is controlled by the one or more genetic loci of interest;
(c) identifying the resulting phenotype of the modified host cell; and
(d) determining that the identified phenotype was the result of the modifications made to the one or more target nucleic acids of interest at the one or more target loci of interest.

To assess the efficiency and/or precision of genome editing (e.g., testing for whether an edit has been made and/or the accuracy of the edit), the target DNA can be analyzed by standard methods known to those in the art. For example, indel mutations can be identified by sequencing using the SURVEYOR® mutation detection kit (Integrated DNA Technologies, Coralville, IA) or the Guide-It™ Indel Identification Kit (Clontech, Mountain View, CA). Homology-directed repair (HDR) can be detected by PCR-based methods, and in combination with sequencing or RFLP analysis. Non-limiting examples of PCR-based kits include the Guide-it Mutation Detection Kit (Clontech) and the GeneArt® Genomic Cleavage Detection Kit (Life Technologies, Carlsbad, CA). Deep sequencing can also be used, particularly for a large number of samples or potential target/off-target sites.

In some other embodiments, editing efficiency can be assessed by employing a reporter or selectable marker to examine the phenotype of an organism or a population of organisms. In some instances, the marker produces a visible phenotype, such as the color of an organism or population of organisms. As a non-limiting example, edits can be made that either restore or disrupt the function of metabolic pathways that confer a visible phenotype (e.g., a color) to the organism. In the scenario where a successful genome edit results in a color change in the target organism (e.g., because the edit disrupts a metabolic pathway that results in a color change or because the edit restores function in a pathway that results in a color change), the absolute number or the proportion of organisms or their progeny that exhibit a color change (e.g., an estimated or direct count of the number of organisms exhibiting a color change divided by the total number of organisms for which the genomes were potentially edited) can serve as a measure of editing efficiency. In some instances, the phenotype is examined by growing the target organisms and/or their progeny under conditions that result in a phenotype, wherein the phenotype may not be visible under ordinary growth conditions.

In some embodiments, the reporter or selectable marker is a fluorescent tagged protein, an antibody, a labeled antibody, a chemical stain, a chemical indicator, or a combination thereof. In other embodiments, the reporter or selectable marker responds to a stimulus, a biochemical, or a change in environmental conditions. In some instances, the reporter or selectable marker responds to the concentration of a metabolic product, a protein product, a synthesized drug of interest, a cellular phenotype of interest, a cellular product of interest, or a combination thereof. A cellular product of interest can be, as a non-limiting example, an RNA molecule (e.g., messenger RNA (mRNA), long non-coding RNA (lncRNA), microRNA (miRNA)).

Editing efficiency can also be examined or expressed as a function of time. For example, an editing experiment can be allowed to run for a fixed period of time (e.g., 24 or 48 hours) and the number of successful editing events in that fixed time period can be determined. Alternatively, the proportion of successful editing events can be determined for a fixed period of time. Typically, longer editing periods will result in a larger number of successful editing events. Editing experiments or procedures can run for any length of time. In some embodiments, a genome editing experiment or procedure runs for several hours (e.g., about 1, 2, 3, 4, 5, 6, 7, 8, 9, 10, 11, 12, 13, 14, 15, 16, 17, 18, 19, 20, 21, 22, 23, or 24 hours). In other embodiments, a genome editing experiment or procedure runs for several days (e.g., about 1, 2, 3, 4, 5, 6, or 7 days).

In addition to the length of time of the editing period, editing efficiency can be affected by the choice of gRNA, donor DNA sequence, the choice of promoter used, or a combination thereof.

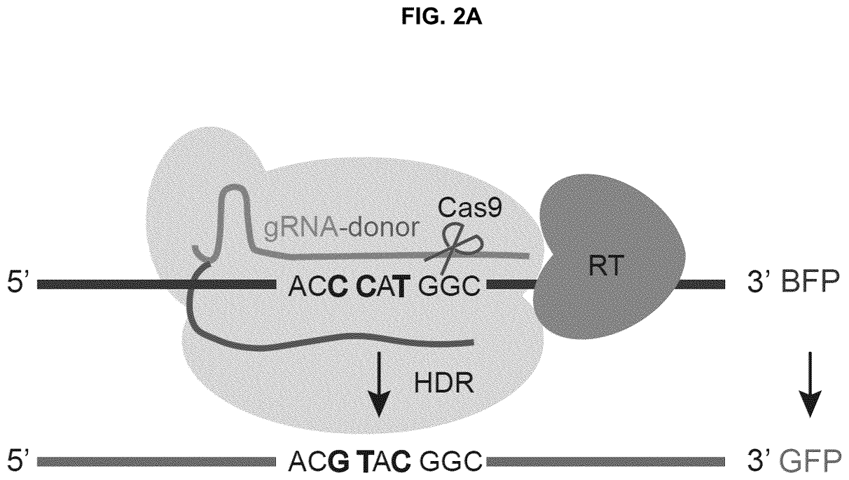

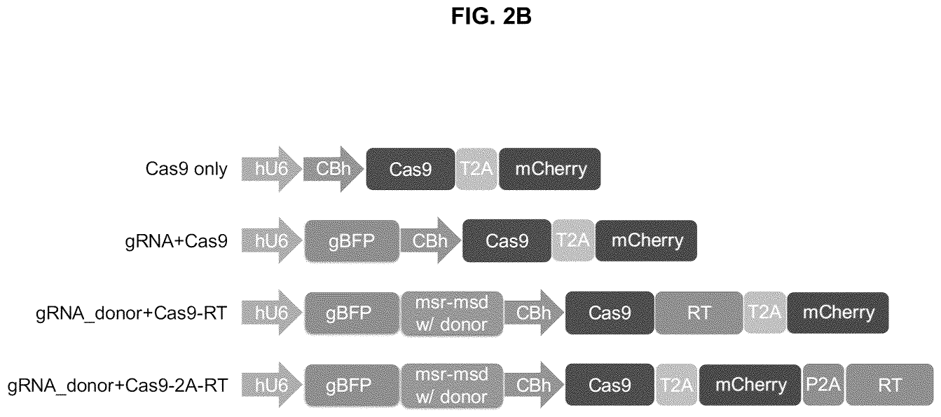

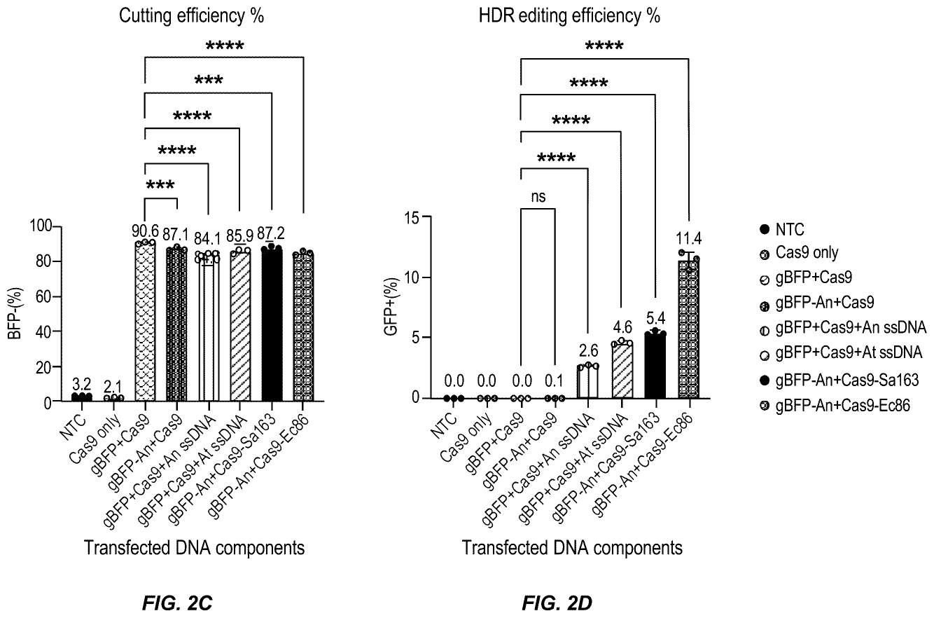

In other embodiments, editing efficiency is compared to a control efficiency. In some embodiments, the control efficiency is determined by running a genome editing experiment in which the retron transcript and gRNA molecule are not coupled. In some instances, the guide RNA (gRNA)-retron cassette is configured such that the transcript products of the gRNA and retron coding region are never physically coupled. In other instances, the retron transcript and gRNA are introduced into the host cell separately. In some instances, the methods and compositions of the present disclosure result in at least about a 1, 2, 3, 4, 5, 6, 7, 8, 9, 10, 20, 30, 40, 50, 60, 70, 80, 90, 100, 150, 200, or more fold increase in efficiency compared to controls, e.g., in cells lacking one or more retron components (see, e.g., FIG. 2D) or when the retron transcript and gRNA are not physically coupled during editing.

Editing efficiency can also be improved by performing editing experiments or procedures in a multiplex format. In some embodiments, multiplexing comprises cloning two or more editing retron-gRNA cassettes in tandem into a single vector. In some instances, at least about 10 retron-gRNA cassettes (i.e., at least about 2, 3, 4, 5, 6, 7, 8, 9, or 10 retron-gRNA cassettes) are cloned into a single vector.

In other embodiments, multiplexing comprises transforming a host cell with two or more vectors. Each vector can comprise one or multiple retron-gRNA cassettes. In some instances, at least about 10 vectors (i.e., at least about 2, 3, 4, 5, 6, 7, 8, 9, or 10 vectors) are used to transform an individual host cell.

In still other embodiments, multiplexing comprises transforming two or more individual host cells, each with a different vector or combination of vectors. In some instances, at least about 2 host cells (i.e., at least about 2, 3, 4, 5, 6, 7, 8, 9, or 10 host cells) are transformed. In other instances, between about 10 and 100 host cells (i.e., about 10, 15, 20, 25, 30, 35, 40, 45, 50, 55, 60, 65, 70, 75, 80, 85, 90, 95, or 100 host cells) are transformed. In still other instances, between about 100 and 1.000 host cells (i.e., about 100, 200, 300, 400, 500, 600, 700, 800, 900, or 1.000 host cells) are transformed. In particular instances, between about 1,000 and 10,000 host cells (i.e., about 1,000, 1,500, 2,000, 2,500, 3.000, 3,500, 4,000, 4,500, 5,000, 5,500, 6,000, 6,500, 7,000, 7,500, 8,000, 8,500, 9,000, 9,500, or 10,000 host cells are transformed). In some other instances, between about 10.000 and 100,000 host cells (i.e., about 10,000, 15,000, 20,000, 25,000, 30.000, 35,000, 40,000, 45,000, 50,000, 55.000, 60,000, 65,000, 70,000, 75,000, 80,000, 85,000, 90,000, 95,000, or 100,000 host cells) are transformed. In other instances, between about 100,000 and 1,000,000 host cells (i.e., at least about 100,000, 150,000, 200.000, 250,000, 300,000, 350.000, 400,000, 450,000, 500.000, 550,000, 600,000, 650,000, 700,000, 750,000, 800,000, 850,000, 900,000, 950,000 or 1,000,000 host cells) are transformed. In some instances, more than about 1,000,000 host cells are transformed. Also, multiple embodiments of multiplexing can be combined.

By using one or a combination of the various multiplexing embodiments, it is possible to modify and/or screen any number of loci within a genome. In some instances, at least about 10 (i.e., about 1, 2, 3, 4, 5, 6, 7, 8, 9, or 10) genetic loci are modified or screened. In other instances, between about 10 and 100 (i.e., about 10, 15, 20, 25, 30, 35, 40, 45, 50, 55, 60, 65, 70, 75, 80, 85, 90, 95, or 100) loci are modified or screened. In still other instances, between about 100 and 1,000 genetic loci (i.e., about 100, 150, 200, 250, 300, 350, 400, 450, 500, 550, 600, 650, 700, 750, 800, 850, 900, 950, or 1,000 genetic loci) are modified or screened. In some other instances, between about 1,000 and 100,000 genetic loci (i.e., about 1,000, 1.500, 2,000, 2.500, 3,000, 3,500, 4,000, 4,500, 5,000, 5,500, 6.000, 6,500, 7.000, 7,500, 8,000, 8,500, 9,000, 9,500, 10,000, 15,000, 20,000, 25,000, 30,000, 35,000, 40,000, 45,000, 50,000, 55,000, 60,000, 65,000, 70,000, 75,000, 80,000, 85,000, 90,000, 95,000, or 100,000 genetic loci) are modified or screened. In particular instances, between about 100,000 and 1,000,000 genetic loci (i.e., about 100,000, 150,000, 200,000, 250,000, 300,000, 350,000, 400,000, 450,000, 500,000, 550,000, 600,000, 650,000, 700,000, 750,000, 800,000, 850,000, 900,000, 950,000, or 1,000,000 genetic loci) are modified or screened. In certain instances, more than about 1,000,000 loci are screened.

In some embodiments, the host cell comprises a population of host cells. In some instances, one or more sequence modifications are induced in at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 percent) of the population of cells.

The precision of genome editing can correspond to the number or percentage of on-target genome editing events relative to the number or percentage of all genome editing events, including on-target and off-target events. Testing for on-target genome editing events can be accomplished by direct sequencing of the target region or other methods described herein. When employing the compositions and methods of the present disclosure, in some instances, editing precision is at least about 1, 2, 3, 4, 5, 6, 7, 8, 9, 10, 11, 12, 13, 14, 15, 16, 17, 18, 19, 20, or more percent, meaning that at least about 1, 2, 3, 4, 5, 6, 7, 8, 9, 10, 11, 12, 13, 14, 15, 16, 17, 18, 19, 20, or more percent of all genome editing events are on-target editing events.

### F. Methods for Preventing or Treating Genetic Diseases

In another aspect, the present disclosure provides a pharmaceutical composition comprising:
(a) a guide RNA (gRNA)-retron cassette of the present disclosure, a Cas9-RT cassette of the present disclosure, a vector of the present disclosure, a gRNA-msr-msd-donor RNA molecule of the present disclosure, a gRNA-donor ssDNA hybrid of the present disclosure, or a combination thereof; and
(b) a pharmaceutically acceptable carrier.

In yet another aspect, provided herein is a method for preventing or treating a genetic disease in a subject, the method comprising administering to the subject an effective amount of a pharmaceutical composition of the present disclosure to correct a mutation in a target gene associated with the genetic disease.

The compositions and methods of the present disclosure are suitable for any disease that has a genetic basis and is amenable to prevention or amelioration of disease-associated sequelae or symptoms by editing or correcting one or more genetic loci that are linked to the disease. Non-limiting examples of diseases include X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism. Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, and ocular diseases. The compositions and methods of the present disclosure can also be used to prevent or treat any combination of suitable genetic diseases.

In some embodiments, the subject is treated before any symptoms or sequelae of the genetic disease develop. In other embodiments, the subject has symptoms or sequelae of the genetic disease. In some instances, treatment results in a reduction or elimination of the symptoms or sequelae of the genetic disease.

In some embodiments, treatment includes administering the herein-disclosed compositions directly to a subject. As a non-limiting example, pharmaceutical compositions as described herein can be delivered directly to a subject (e.g., by local injection or systemic administration). In other embodiments, the compositions are delivered to a host cell or population of host cells, and then the host cell or population of host cells is administered or transplanted to the subject. The host cell or population of host cells can be administered or transplanted with a pharmaceutically acceptable carrier. In some instances, editing of the host cell genome has not yet been completed prior to administration or transplantation to the subject. In other instances, editing of the host cell genome has been completed when administration or transplantation occurs. In certain instances, progeny of the host cell or population of bost cells are transplanted into the subject. In some embodiments, correct editing of the host cell or population of host cells, or the progeny thereof, is verified before administering or transplanting edited cells or the progeny thereof into a subject. Procedures for transplantation, administration, and verification of correct genome editing are discussed herein and will be known to one of skill in the art.

Compositions of the present disclosure, including cells and/or progeny thereof that have had their genomes edited by the present methods and/or compositions, may be administered as a single dose or as multiple doses, for example two doses administered at an interval of about one month, about two months, about three months, about six months or about 12 months. Other suitable dosage schedules can be determined by a medical practitioner.

Prevention or treatment can further comprise administering agents and/or performing procedures to prevent or treat concomitant or related conditions. As non-limiting examples, it may be necessary to administer drugs to suppress immune rejection of transplanted cells, or prevent or reduce inflammation or infection. A medical professional will readily be able to determine the appropriate concomitant therapies.

### G. Kits

In another aspect, the present disclosure provides kit for modifying one or more target nucleic acids of interest at one or more target loci within a genome of a host cell, the kit comprising one or a plurality of vectors of the present disclosure. The kit may further comprise a host cell or a plurality of host cells.

In some embodiments, the kit contains one or more reagents. In some instances, the reagents are useful for transforming a host cell with a vector or a plurality of vectors, and/or inducing expression from the vector or plurality of vectors. In other embodiments, the kit may further comprise a reverse transcriptase, a plasmid for expressing a reverse transcriptase, one or more nucleases, one or more plasmids for expressing one or more nucleases, or a combination thereof. The kit may further comprise one or more reagents useful for delivering nucleases or reverse transcriptases into the host cell and/or inducing expression of the reverse transcriptase and/or the one or more nucleases. In yet other embodiments, the kit further comprises instructions for transforming the host cell with the vector, introducing nucleases and/or reverse transcriptases into the host cell, inducing expression of the vector, reverse transcriptase, and/or nucleases, or a combination thereof.

In yet another aspect, the present disclosure provides a kit for modifying one or more target nucleic acids of interest at one or more target loci within a genome of a host cell, the kit comprising one or a plurality of retron donor DNA-guide molecules of the present disclosure. The kit may further comprise a host cell or a plurality of host cells.

In some embodiments, the kit contains one or more reagents. In some instances, the reagents are useful for introducing one or more of the cassettes, transcripts, RNA-DNA hybrids, fusion proteins, or vectors into the host cell. The kit may further comprise one or more reagents useful for inducing expression of any of the herein-described cassettes. In yet other embodiments, the kit further comprises instructions for introducing one or more of the cassettes, transcripts, RNA-DNA hybrids, fusion proteins, or vectors into a mammalian host cell, for inducing expression of the gRNA-donor template transcript or Cas9-RT fusion, or a combination thereof.

### H. Applications

The compositions and methods provided by the present disclosure are useful for any number of applications. As non-limiting examples, genome editing can be performed to correct detrimental lesions in order to prevent or treat a disease, or to identify one or more specific genetic loci that contribute to a phenotype, disease, biological function, and the like. As another non-limiting example, genome editing or screening according to the compositions and methods of the present disclosure can be used to improve or optimize a biological function, pathway, or biochemical entity (e.g., protein optimization). Such optimization applications are especially suited to the compositions and methods of the present disclosure, as they can require the modification of a large number of genetic loci and subsequently assessing the effects.

Other non-limiting examples of applications suitable for the herein-disclosed compositions and methods include the production of recombinant proteins for pharmaceutical and industrial use, the production of various pharmaceutical and industrial chemicals, the production of vaccines and viral particles, and the production of fuels and nutraceuticals. All of these applications typically involve high-throughput or high-content screening, making them especially suited to the compositions and methods described herein.

In some embodiments, inducing one or more sequence modifications at one or more genetic loci of interest comprises substituting, inserting, and/or deleting one or more nucleotides at the one or more genetic loci of interest. In some instances, inducing the one or more sequence modifications results in the insertion of one or more sequences encoding cellular localization tags, one or more synthetic response elements, and/or one or more sequences encoding degrons into the genome.

In other embodiments, inducing the one or more sequence modifications at the one or more genetic loci of interest results in the insertion of one or more sequences from a heterologous genome. Introducing heterologous DNA sequences into a genome is useful for any number of applications, some of which are described herein. Others will be readily apparent to one of skill in the art. Non-limiting examples are directed protein evolution, biological pathway optimization, and production of recombinant pharmaceuticals.

In certain embodiments, inducing the one or more sequence modifications at the one or more genetic loci of interest results in the insertion of one or more “barcodes” (i.e., nucleotide sequences that allow identification of the source of a particular specimen or sample). As non-limiting examples, the insertion of barcodes can be used for cell lineage tracking or the measurement of RNA abundance.

In addition to gene editing, the present methods can be used for numerous other applications, based on the ability of the methods to generate ssDNA in human cells via retron activity. For example, in some embodiments, the present methods and compositions are used to generate single-stranded DNA in human cells for DNA origami (34). In some embodiments, the present methods and compositions are used to generated single-stranded DNA in human cells for genome modification, e.g., via intrachromosomal recombination (35). In some embodiments, the present methods and compositions are used to generated single-stranded DNA in human cells to produce oligonucleotides that can fold into 3D structures that bind target molecules (i.e., aptamers) (36, 37).

### V. Examples

### Example 1. Bacterial Retrons Enable Precise Gene Editing in Human Cells

### Abstract

Retrons are bacterial genetic elements involved in anti-phage defense. They have the unique ability to reverse transcribe RNA into multicopy single-stranded DNA (msDNA) that remains covalently linked to their template RNA. Retrons coupled with CRISPR-Cas9 in yeast have been shown to improve editing efficiency of precise genome editing via homology-directed repair (HDR). HDR editing efficiency has been limited by challenges associated with delivering extracellular donor DNA encoding the desired mutation. In this study, we tested the ability of retrons to produce msDNA as donor DNA and facilitate HDR by tethering msDNA to guide RNA in HEK293T and K562 cells. Through heterologous reconstitution of retrons from multiple bacterial species with the CRISPR-Cas9 system, we demonstrated HDR rates of up to 11.4%. Overall, our findings represent the first step in extending retron-based precise gene editing to human cells.

### Introduction

We have previously demonstrated that retron-derived msDNA can facilitate template-mediated precise genome editing in yeast 14. Cas9-Retron precISe Parallel Editing via homologY (CRISPEY) utilizes retrons to produce donor DNA molecules and vastly improves HDR editing efficiency to ˜96%, allowing for the characterization of the fitness effects of over 16,000 natural genetic variants at single-base resolution. A similar strategy utilizing Cas9, retrons, and single-stranded DNA binding proteins has been demonstrated in bacteria (15), in which msDNA can be incorporated into the bacterial genome without the aid of targeted nucleases (16). Importantly, a previous study in mouse 3T3 cells provided evidence that msDNA can be produced in mammalian cells at very low amounts (17). Hence, we envisioned that retron-generated msDNA could be harnessed for precise gene editing in human cells (FIG. 1A).

### Materials and Methods

### Retron Plasmids

DNA sequences for retrons, primers, and plasmids used in this study are listed in the Informal Sequence Listing. Genes encoding SpCas9 and BFP were obtained from previously reported plasmids (Addgene plasmid #64323, #64216, #64322, Ralf Kühn lab (18); mCherry was amplified from Addgene plasmid #60954, Jonathan Weissman lab (19)). Retron genes were synthesized as gBlocks Gene Fragments (Integrated DNA Technologies) or clonal genes (Twist Bioscience). GFP donor genes were synthesized as gBlocks Gene Fragments (Integrated DNA Technologies). Primers and gRNA were synthesized as oligos (Integrated DNA Technologies). The parental vector (Addgene plasmid #64323, Ralf Kühn's lab) was digested by restriction endonucleases (New England Biolabs). The digested vector backbone was purified using Monarch DNA Gel Extraction Kit (New England Biolabs) or NucleoSpin Gel and PCR Clean-up Kit (Macherey-Nagel), gRNA targeting BFP (gBFP) was inserted with Golden Gate cloning. PCR was performed using Q5 High-Fidelity DNA Polymerase or Q5 High-Fidelity 2× Master mix (New England Biolabs). PCR products were purified using Monarch PCR & DNA Cleanup Kit (New England Biolabs). RT, msr-msd, and donors were inserted into the digested vector backbone with Gibson Assembly using NEBuilder HiFi DNA Assembly Master Mix (New England Biolabs) Donors were replaced via double digestion by SpeI and AvrII (New England Biolabs). Plasmids were amplified using Stbl3 competent cells prepared with The Mix & Go! E. coli Transformation Kit and Buffer Set (Zymo Research) and extracted by the Plasmid Plus Midi Kit (Qiagen) following the manufacturer's protocol. Extracted plasmids were measured by Nanodrop (Thermo Fisher Scientific), normalized to the same concentration, and subsequently validated by Sanger sequencing.

### Cell Lines and Culture

HEK293T BFP and K562 BFP reporter cells were provided by Dr. Jacob Corn (ETH Zürich) and Dr. Christopher D Richardson (UCSB). K562 wildtype cells were provided by Dr. Stanley Qi's group (Stanford). HEK293T wildtype and HEK293T BFP reporter cells were maintained in Dulbecco's Modified Eagle's Medium (DMEM) with GlutaMax (Thermo Fisher Scientific) supplemented with 10% v/v fetal bovine serum (FBS) (Gibco) and 10% penicillin-streptomycin (Thermo Fisher Scientific). K562 wildtype and K562 BFP reporter cells were maintained in PRMI 1640 (Thermo Fisher Scientific) supplemented with 10% v/v FBS (Gibco) and 10% penicillin-streptomycin (Thermo Fisher Scientific). All cells were maintained at 37° C. with 5% CO2.

### Transfection

All dilutions and complex formations used Opti-MEM Reduced Serum Medium (Thermo Fisher Scientific). For transfection of one well in a 48-well Poly-D-Lysine coated plate (Corning), 1 μg plasmids were mixed with transfection reagents using Lipofectamine 3000 (Life Technologies) according to the manufacturer's protocol with the following modifications: after incubating for 30 minutes at room temperature, the DNA-reagent complex was added to each well; then 200.000 HEK293T BFP reporter cells in 200 μL DMEM media were added into each well and mixed gently with the DNA-transfection reagents.

The Neon™ Transfection System 10 μL kit (Thermo Fisher Scientific) was used to transfect K562 and K562 BFP reporter cells according to the manufacturer's protocol. Cells were washed in Dulbecco's phosphate-buffered saline (DPBS) (Thermo Fisher Scientific), transfected with 1 μg plasmids at 1050v/20 ms/2 pulses and cultured in a 24-well Nunc cell culture plate (Thermo Fisher Scientific) at the density of 200,000/well in PRMI 1640 (Thermo Fisher Scientific) supplemented with 10% FBS (Gibco).

qPCR

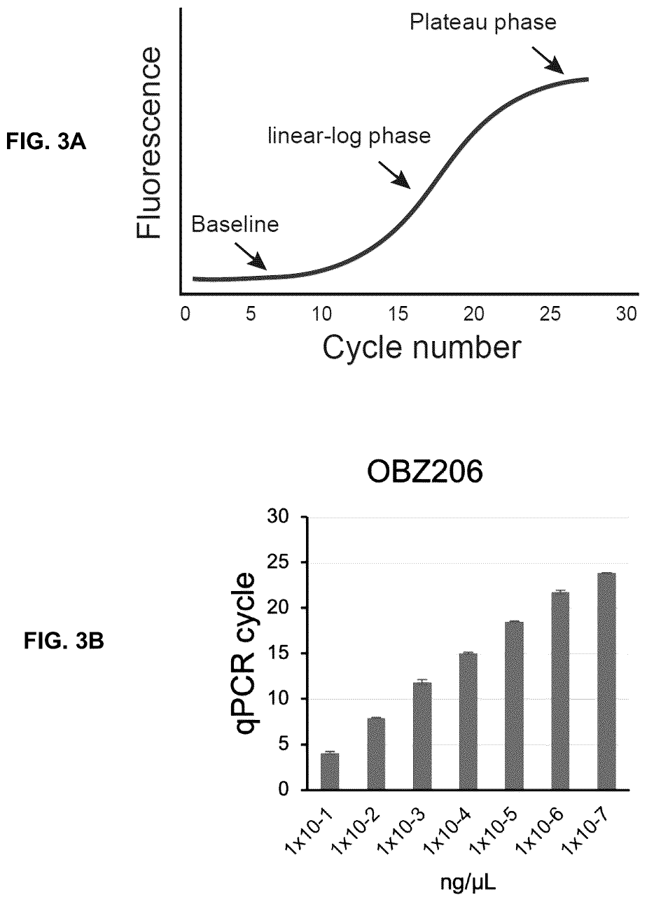

qPCR assay was performed 72 hours post-transfection. K562 cells were spun down at 1,000 rpm for 5 mins. Then, cell pellets were washed in DPBS. Cell pellets were harvested after being spun down again at 1,000 rpm for 5 mins. The gRNA-msDNA hybrid was extracted with the QuickExtract RNA Extraction Solution (Lucigen) according to the manufacturer's protocol. The extract was digested using double-stranded DNase (Thermo Fisher Scientific). The digested product was purified by using the ssDNA/RNA Clean & Concentrator Kit (Zymo Research). The purified product was then used as the qPCR template, qPCR primers are listed in Supplemental Note 1. The qPCR assay was carried out using iQ SYBR Green Supermix (Bio-Rad), qPCR data was collected on the CFX384 Touch™ Real-Time PCR Detection System (Bio-Rad). We performed a sequential 10× dilution and used this ssDNA as a measurement standard to generate a series of positive signals, which reflected the slope of log-linear regions in a qPCR assay (FIGS. 3A-3B). The qPCR conditions are shown in Tables 1A-1B.

TABLE 1A
qPCR Conditions

| | Volume |
|---|---|
| Component | (μl) |
| Total | 10 |
| H2O | 3.4 |
| iQTM SYBR ® Green supermix (2x) | 5 |
| OBZ152 + OBZ153 (5 μM each) | 0.6 |
| extracted ssDNA | 1 |

TABLE 1B
qPCR Cycling Steps

| | | |
|---|---|---|
| Temperature (° C.) | Time | Cycles |

| | | | |
|---|---|---|---|
| 95 | 3 | min | 1 |
| 95 | 10 | s | 40 |
| 55 | 30 | s | |

### Flow Cytometry

293T BFP and K562 BFP reporter cells were washed in DPBS (Thermo Fisher Scientific), resuspended in DPBS (Thermo Fisher Scientific), supplemented with 5% v/v FBS (Gibco) at a concentration of 1,000,000 cells/mL, and measured via flow cytometry. Transfected cells were isolated by using a red fluorescence protein mCherry, which was co-expressed with Cas9 and RT. Editing outcomes were recorded 72 hours, 96 hours and 7 days post-transfection or electroporation on an Attune NxT flow cytometer (Invitrogen). For 293T BFP reporter cells, data at 72 hours are presented in the figures. For K562 BFP reporter cells, data at 96 hours are presented in the figures. All plots were analyzed using FlowJo v 10.7.1.

### Results

Retrons Produce msDNA in Human Cells

To implement the CRISPEY strategy in human cells, we first tested whether msDNA can be produced in human cells. To maximize the chance of msDNA production, we estimated the expression of multiple candidate retrons. To date, hundreds of putative retrons have been characterized by computational analyses, among which 16 were experimentally validated to produce msDNA via in vitro assays (9). We codon-optimized eight fully annotated RTs with publically available protein sequences and synthesized the corresponding retron RNA to enable heterologous expression in human cells (see, e.g., Table 2. Materials and Methods, Informal Sequence Listing). Since the biosynthesis of msDNA requires both msr-msd and RT, we combined the sequences of msr-msd and RT with SpCas9/sgRNA into a single multicistronic vector. We drove the transcription of the chimeric sgRNA-msr-msd by the RNA polymerase III promoter U6 and embedded the donor DNA templates in the replaceable regions within msd sequences (FIG. 1B). To measure the total free gRNA-msDNA product, we utilized a gRNA targeting mouse gene Rosa26 and re-constructed the CRISPR-Cas9 vectors that were established in Chu et al. (18) (See Materials and Methods). We drove the co-expression of RT and SpCas9 by the CBh promoter, an RNA polymerase II promoter that provides robust and long-term expression (20). We engineered Cas9-RT fusion proteins to test whether spatial colocalization of these proteins improved their editing efficiency. In parallel, we employed the 2A self-cleaving peptide-based multi-gene expression system to co-express Cas9 and RT (21) in case the functions of Cas9 and/or RT were impeded by steric hindrance.

TABLE 2
Summary of Retrons Used in This Study

| Retron | Species | Bacterial taxon | Reference | Source |
|---|---|---|---|---|
| Sa163 | Stigmatella | Deltaproteobacteria | Yee et al. 1984 (11) | NCBI RefSeq: |
| | aurantiaca | | | ZP 01463804.1 |
| Ec86 | Escherichia coli | Gammaproteobacteria | Lim & Maas 1989 (38) | Uniprot: P23070 |
| Mx65 | Myxococcus xanthus | Deltaproteobacteria | Inouye et al. 1990 (39) | Uniprot: P23071 |
| Mx162 | Myxococcus xanthus | Deltaproteobacteria | Yee et al. 1984 (11) | Uniprot: P23072 |
| Ec73 | Escherichia coli | Gammaproteobacteria | Sun et al. 1991 (40) | Uniprot: Q7M2A9 |
| Ec107 | Escherichia coli | Gammaproteobacteria | Herzer et al. 1992 (41) | Uniprot: Q05804 |
| St85 | Salmonella enterica | Gammaproteobacteria | Ahmed & Shimamoto | GenBank: |
| | Typhimurium | | 2003 (42) | ACY91017.1 |
| Ec67 | Escherichia coli | Gammaproteobacteria | Lampson et al. 1989 (43) | Uniprot: P21325 |

Adapted from Simon et al. (9). Sequences are shown in the Informal Sequence Listing.

To test whether retrons can generate msDNA in human cells, we transfected the multicistronic plasmids into human K562 cells and measured msDNA by qPCR. Since bacterial retron products are normally absent in human cells, we synthesized a single-stranded DNA (ssDNA) as the standard template, which is the same as the DNA donor template inserted into the plasmids (see, e.g., Informal Sequence Listing). A summary of retrons that generated msDNA in K562 cells are summarized in the Informal Sequence Listing. Collectively, of the eight retrons, Sa163 showed the highest msDNA production activity under the conditions tested (FIG. 1C). While several other retrons had higher msDNA production than retron Ec86, we previously validated retron Ec86-mediated CRISPEY in yeast (14). These studies and our results suggested that both Sa163 and Ec86 possessed the potential to be explored as tools for precise gene editing and were selected for further study.

### Retron Ec86 and Sa163 Enable HDR in Both Suspension and Adherent Human Cell Lines

To test if retrons can promote HDR, we used the reporter cell lines previously described in Richardson et al. (22) that used BFP-to-GFP conversions as editing readout. When HDR occurs, a three-nucleotide substitution converts the integrated BFP reporter into GFP (FIG. 2A). We co-expressed the red fluorescence protein mCherry with Cas9 and RT in the reporter line and used the multicistronic retron plasmid to generate donors to convert BFP to GFP (FIG. 2B). After inducing edits for each retron, we isolated transfected cells by flow cytometry to evaluate HDR (FIG. 4A). We used the BFP-GFP donor template to convert the protein expression from BFP to GFP.

Since previous studies have shown that donor DNA length and strand type (e.g., target vs. non-target) influence editing (22), we tested three pairs of gRNA target and non-target-strand donor templates (FIG. 4B). As illustrated in FIG. 1A, we transfected the all-in-one plasmid carrying gRNA, donor, Cas9, and retron coding sequence. Thus, compared to the conventional approach of ribonucleoprotein (RNP) delivery, gRNA was produced in cells after transfection, while single-stranded donor DNA was generated via a two-step process of transcription and reverse transcription.

In general, higher HDR rates were detected when using donors that were complementary to the non-target strand (FIGS. 4C-4E), which was consistent with previous studies showing that Cas9 first releases the non-target strand after cleavage (22). Although free Sa163 produced in excess of two-fold more msDNA than Cas9-Sa163 RT fusion (Cas9-2A-Sa163 vs. Cas9-Sa163; FIG. 1C), HDR editing by Cas9-Sa163 fusion was 3.3-fold higher (gBFP-An+Cas9-Sa163 vs. gBFP-An+Cas9-2A-Sa163; FIG. 4D). Having Sa163 RT fused to Cas9 may increase HDR by producing msDNA in the vicinity of the double-strand breaks (DSBs), even though the free Sa163 RT can produce more msDNAs that are globally distributed in the nucleus.

Taken together, our findings indicated that retrons Ec86 and Sa163 can facilitate HDR in both K562 and HEK293T cells (FIGS. 4C-4E).

After Cas9-induced DSBs, the BFP expression was disrupted, and the BFP+ cells were converted into BFP− cells. Therefore, we counted BFP− cells to determine Cas9 cutting efficiency (though this is likely to be an underestimate, since some NHEJ outcomes will not lead to loss of BFP). CRISPR-Cas9 cutting efficiency in human cells (90.6%; FIG. 2C) was comparable to efficiency when either retron was co-expressed (87.2% for Sa163, 84.6% for Ec86; FIG. 2C). This finding suggests that our gRNA-msDNA hybrid did not severely impact the gRNA recognition and that our Cas9-RT fusion proteins did not suppress Cas9's nuclease activity.

In K562 BFP reporter cells, controls without retron components (gBFP-An+Cas9) had only 0.1% HDR events, whereas Ec86 yielded 11.4% HDR events (FIG. 2D). Sa163, the retron with the highest level of msDNA production (FIG. 1C), had 5.4% HDR events (FIG. 2D). By comparison, cells co-transfected with plasmid expressing gBFP and Cas9, and in vitro synthesized ssDNA oligos targeting each strand of the BFP target region (gBFP+Cas9-An ssDNA, gBFP+Cas9-At ssDNA), had mean values of 2.6% and 4.6%, respectively (FIG. 2D). This comparison suggests that the ssDNA donors generated by retrons can be more effective in promoting HDR than exogenously delivered ssDNA donors.

### Discussion

Retrons are unique bacterial DNA elements that are capable of generating msDNA in vivo through reverse transcription. Recently, two independent groups have reported the role of retrons in antiphage defense in prokaryotes (23, 24). We hypothesized that retron-generated msDNA could be utilized to generate repair templates for precise genome editing in human cells. Here, we showed that: (1) retrons from different bacterial species have a wide range of RT activity in human cells and (2) simultaneous expression of retron RT with a hybrid retron RNA/sgRNA transcript can facilitate precise editing in HEK293 and K562 cells. Building on our previous study of retron Ec86 in yeast (14), our results suggest that both retron Ec86 and Sa163 may enable precise gene editing in human cells.

Increasing the supply of donor template at the editing site has been shown to improve the efficiency of HDR repair in yeast and mammalian cells (25, 26). The CRISPEY gRNA-retron design allows the sgRNA and msDNA to be covalently linked, which is intended to make the donor template immediately available for HDR repair at Cas9-induced DSBs. Further improvement of the retron RT processivity may generate more gRNA/msDNA hybrids available for recruitment or simply increase the donor template concentration in the nucleus to increase the probability of HDR over NHEJ. Similarly, the retron RNA scaffold can also be engineered to provide increased affinity for RT binding or activity. Both the retron RT and retron RNA can be engineered through directed evolution or knowledge-based enzyme variant design, such as that seen with group II intron RTs (27).

We have explored several experimentally validated retrons for msDNA production. Notably, Sa163 generated the most msDNA in K562 cells, as measured by qPCR (FIG. 1C). However, Sa163 had a lower HDR rate when compared to Ec86 in the BFP-GFP reporter (FIG. 2D). This discrepancy between donor abundance and HDR rate raises the question of whether higher donor concentration increases HDR editing. One possible explanation for this discrepancy is post-transfection cell toxicity that may occur with high retron activity, as suggested by decreasing expression of mCherry in the cell population over time. Although Sa163 produced more msDNA, the increased toxicity may be contributing to reduced cellular viability in cells with higher RT and msDNA expression, resulting in fewer HDR-edited cells. Therefore, future work may survey retron species for efficient generation of msDNA with lower toxicity, which may also improve CRISPEY editing efficiency without the necessity of engineering of RT and retron RNA.

Many potential avenues exist for further optimization of CRISPEY in humans or other species. For example, single-strand annealing proteins (SSAPs) have been shown as effector proteins to improve retron-mediated editing in bacteria (15, 16, 28). More recently, there is evidence that SSAPs also improve efficiency of Cas9-mediated knock-ins in human cells (29). It is thus tempting to speculate that co-expression of SSAP may further improve CRISPEY editing efficiency.

Recent advances in base editors (30-32) and prime editors (33) have led to highly efficient editing rates. However, these methods are limited to short genetic alterations. In contrast. CRISPEY can efficiently insert gene-length fragments (e.g., GFP) in yeast (14). This approach may expand the length of potential knock-ins in human cells by circumventing the need to deliver long donor DNA molecules.

Beyond precise gene editing, there are other promising applications of retron activity in human cells for generating ssDNA. Generation of ssDNA is of great interest due to its use in biotechnology (e.g., DNA origami) (34), genome modification (e.g., intrachromosomal recombination) (35), and generation of single stranded oligonucleotides that can fold into 3D structures that bind target molecules (i.e., aptamers) (36, 37).

### Conclusion

In this study, we harnessed the capability of retrons to generate intracellular gRNA-msDNA hybrid molecules and repurposed the products for CRISPR-Cas9-mediated, homology-directed repair in human cells. We presented a precise genome editing method that provides unique advantages in donor delivery, especially for repair templates that are difficult to deliver via conventional approaches. With further optimization, the msDNA generated by retrons has vast potential in precise gene editing and other biotechnology applications.

### VI. Exemplary Embodiments

Exemplary embodiments provided in accordance with the presently disclosed subject matter include, but are not limited to, the claims and the following embodiments:
1. A guide RNA (gRNA)-retron cassette for use in genomic editing in a mammalian cell comprising:
(a) a gRNA coding region, wherein the target sequence of the gRNA is within a mammalian genetic locus; and
(b) a retron region comprising:
   (i) an msr locus;
   (ii) a first inverted repeat sequence;
   (iii) an msd locus;
   (iv) a donor DNA template region located within the msd locus, wherein the donor DNA template comprises homology to one or more sequences within the mammalian genetic locus; and
   (v) a second inverted repeat sequence,
wherein the gRNA coding region is upstream of the retron region in the cassette such that transcription of the cassette results in a transcript in which the gRNA is 5′ of the RNA transcribed from the retron region.
2. The cassette of embodiment 1, wherein the first inverted repeat sequence is located within the 5′ end of the msr locus.
3. The cassette of embodiment 1 or 2, wherein the second inverted repeat sequence is located 3′ of the msd locus.
4. The cassette of any one of embodiments 1 to 3, wherein the retron region encodes an RNA molecule that is capable of self-priming reverse transcription by a reverse transcriptase (RT).
5. The cassette of embodiment 4, wherein reverse transcription of the RNA molecule produces a hybrid molecule that comprises RNA and single stranded DNA (ssDNA), wherein the RNA within the hybrid molecule comprises the gRNA, wherein the ssDNA within the hybrid molecule comprises the donor DNA template, and wherein the gRNA and donor DNA template are covalently linked.
6. The cassette of any one of embodiments 1 to 5, wherein the donor DNA template comprises two homology arms, wherein each homology arm has at least about 70% to about 99% similarity to a portion of the mammalian genetic locus on either side of the gRNA target sequence.
7. A vector comprising the cassette of any one of embodiments 1 to 6.
8. The vector of embodiment 7, further comprising a promoter that is operably linked to the cassette.
9. The vector of embodiment 8, wherein the promoter is an RNA polymerase III (Pol III) promoter
10. The vector of embodiment 9, wherein the msd locus comprises one or more sequence modifications to avoid pre-mature Pol III termination.
11. The vector of embodiment 10, wherein the one or more sequence modifications comprise single nucleotide substitutions.
12. The vector of embodiment 10 or 11, wherein the msd locus comprises a “TTTT” to “TTTc” or “TTTa” sequence modification in the stem region.
13. The vector of embodiment 12, wherein the msd locus further comprises a modification of a corresponding sequence in the opposite strand of the stem region for maintaining secondary structure.
14. The vector of embodiment 13, wherein the modification of the corresponding sequence comprises a “GGAAA” to “GGgAA” sequence modification or a “GAAAA” to “GgAAA” sequence modification.
15. The vector of any one of embodiments 10 to 14, wherein the msd locus further comprises a “TTTTTT” to “TTTETT” sequence modification downstream of the stem region.
16. The vector of any one of embodiments 10 to 15, wherein the msd locus comprises an Ec86 msd sequence.
17. The vector of any one of embodiments 7 to 16, further comprising a second cassette comprising a coding sequence for a fusion protein comprising an RNA-guided nuclease and a reverse transcriptase (RT).
18. The vector of any one of embodiments 7 to 16, further comprising a second cassette comprising a coding sequence for a bicistronic polypeptide comprising an RNA-guided nuclease and a reverse transcriptase (RT), separated by a self-cleaving peptide.
19. The vector of embodiment 18, wherein the self-cleaving peptide is T2A or P2A.
20. The vector of any one of embodiments 17 to 19, wherein the coding sequence is codon optimized for mammalian cells.
21. The vector of any one of embodiments 17 to 20, wherein the RNA-guided nuclease is saCas9, spCas9, or Cpf1.
22. The vector of any one of embodiments 17 to 21, further comprising a promoter operably linked to the second cassette.
23. The vector of embodiment 22, wherein the promoter operably linked to the second cassette is an RNA polymerase II (Pol II) promoter.
24. A gRNA-msr-msd-donor RNA molecule for use in genomic editing in a mammalian cell comprising:
(a) a guide RNA (gRNA), wherein the target sequence of the gRNA is within a mammalian genetic locus; and
(b) a retron transcript comprising:
   (i) an msr region;
   (ii) a first inverted repeat sequence;
   (iii) an msd region;
   (iv) a donor DNA template coding region located within the msd region, wherein the encoded donor DNA template comprises homology to the mammalian genetic locus; and
   (v) a second inverted repeat sequence.
25. The gRNA-msr-msd-donor RNA molecule of embodiment 24, wherein the first inverted repeat sequence is located within the 5′ end of the msr region.
26. The gRNA-msr-msd-donor RNA molecule of embodiment 24 or 25, wherein the second inverted repeat sequence is located 3′ of the msd region.
27. The gRNA-msr-msd-donor RNA molecule of any one of embodiments 24 to 26, wherein the retron transcript is capable of self-priming reverse transcription by a reverse transcriptase (RT).
28. The gRNA-msr-msd-donor RNA molecule of any one of embodiments 24 to 27, wherein the gRNA is 5′ of the retron transcript.
29. The gRNA-msr-msd-donor RNA molecule of any one of embodiments 24 to 28, wherein reverse transcription of the retron transcript produces a hybrid molecule that comprises RNA and single stranded DNA (ssDNA), wherein the RNA within the hybrid molecule comprises the gRNA, wherein the ssDNA comprises the donor DNA template, and wherein the gRNA and donor DNA template are covalently linked.
30. The gRNA-msr-msd-donor RNA molecule of any one of embodiments 24 to 29, wherein the donor DNA template coding region comprises sequences encoding two homology arms, wherein each homology arm has at least about 70% to about 99% similarity to a portion of the mammalian genetic locus on either side of the gRNA target sequence.
31. A method for modifying one or more target nucleic acids of interest at one or more target loci within a genome of a mammalian host cell, the method comprising:
(a) transforming the mammalian host cell with a vector of any one of embodiments 7 to 23; and
(b) culturing the host cell or transformed progeny of the host cell under conditions sufficient for expressing from the vector a gRNA-msr-msd-donor RNA molecule,
wherein the retron transcript within the gRNA-msr-msd-donor RNA molecule self-primes reverse transcription by a reverse transcriptase (RT) expressed by the host cell or the transformed progeny of the host cell,
wherein at least a portion of the retron transcript is reverse transcribed to produce a hybrid molecule that comprises RNA and single stranded DNA (ssDNA),
wherein the RNA within the hybrid molecule comprises the gRNA,
wherein the ssDNA within the hybrid molecule comprises the donor DNA template,
wherein the gRNA and donor DNA template are covalently linked.
wherein the donor DNA template comprises homology to the one or more target loci and comprises sequence modifications compared to the one or more target nucleic acids,
wherein the one or more target loci are cut by an RNA-guided nuclease expressed by the host cell or transformed progeny of the host cell,
wherein the reverse transcriptase and the RNA-guided nuclease are present within a single fusion protein or a bicistronic polypeptide separated by a self-cleaving peptide,
wherein the site of cutting by the RNA-guided nuclease is determined by the target sequence of the gRNA, and
wherein the one or more donor DNA template sequences recombine with the one or more target nucleic acid sequences to insert, delete, and/or substitute one or more bases of the sequence of the one or more target nucleic acid sequences to induce one or more sequence modifications at the one or more target loci within the genome.
32. The method of embodiment 31, wherein the msr and msd regions of the retron transcript form a secondary structure, wherein the formation of the secondary structure is facilitated by base pairing between the first and second inverted repeat sequences, and wherein the secondary structure is recognized by the RT for the initiation of reverse transcription.
33. The method of embodiment 31 or 32, wherein the RNA-guided nuclease is saCas9, spCas9, or Cpf1.
34. The method of any one of embodiments 31 to 33, wherein the self-cleaving peptide is T2A or P2A.
35. The method of any one of embodiments 31 to 34, wherein the one or more donor DNA sequences comprise two homology arms, wherein each homology arm has at least about 70% to about 99% similarity to a portion of the mammalian genetic locus on either side of the gRNA target sequence.
36. The method of any one of embodiments 31 to 35, wherein the isolated mammalian host cell is a human cell.
37. The method of any one of embodiments 31 to 36, wherein about ten or more target loci are modified.
38. The method of any one of embodiments 31 to 37, wherein the host cell comprises a population of host cells.
39. The method of any one of embodiments 31 to 38, further comprising introducing a single-strand annealing protein (SSAP) into the host cell.
40. A method for screening one or more genetic loci of interest in a genome of a mammalian host cell, the method comprising:
(a) modifying one or more target nucleic acids of interest at one or more target loci within the genome of the host cell according to the method of any one of embodiments 31 to 39;
(b) incubating the modified host cell under conditions sufficient to elicit a phenotype that is controlled by the one or more genetic loci of interest;
(c) identifying the resulting phenotype of the modified host cell; and
(d) determining that the identified phenotype was the result of the modifications made to the one or more target nucleic acids of interest at the one or more target loci of interest.
41. The method of embodiment 40, wherein at least 1,000 to 1,000,000 genetic loci of interest are screened simultaneously.
42. The method of embodiment 40, wherein the phenotype is identified using a reporter.
43. The method of embodiment 42, wherein the reporter is selected from the group consisting of a fluorescent tagged protein, an antibody, a chemical stain, a chemical indicator, and a combination thereof.
44. The method of embodiment 42, wherein the reporter responds to the concentration of a metabolic product, a protein product, a synthesized drug of interest, a cellular phenotype of interest, or a combination thereof.
45. A mammalian host cell that has been transformed by a vector of any one of embodiments 7 to 23.
46. A pharmaceutical composition comprising:
(a) the guide RNA-retron cassette of any one of embodiments 1 to 6, the vector of any one of embodiments 7 to 23, the gRNA-msr-msd-donor RNA molecule of any one of embodiments 24 to 30, or a combination thereof; and
(b) a pharmaceutically acceptable carrier.
47. A method for preventing or treating a genetic disease in a subject, the method comprising administering to the subject an effective amount of the pharmaceutical composition of embodiment 46 to correct a mutation in a target gene associated with the genetic disease.
48. The method of embodiment 47, wherein the genetic disease is selected from the group consisting of X-linked severe combined immune deficiency, sickle cell anemia, thalassemia, hemophilia, neoplasia, cancer, age-related macular degeneration, schizophrenia, trinucleotide repeat disorders, fragile X syndrome, prion-related disorders, amyotrophic lateral sclerosis, drug addiction, autism, Alzheimer's disease, Parkinson's disease, cystic fibrosis, blood and coagulation diseases and disorders, inflammation, immune-related diseases and disorders, metabolic diseases and disorders, liver diseases and disorders, kidney diseases and disorders, muscular/skeletal diseases and disorders, neurological and neuronal diseases and disorders, cardiovascular diseases and disorders, pulmonary diseases and disorders, ocular diseases and disorders, and a combination thereof.
49. A kit for modifying one or more target nucleic acids of interest at one or more target loci within a genome of a host cell, the kit comprising one or a plurality of vectors of any one of embodiments 7 to 23.
50. The kit of embodiment 49, further comprising a mammalian host cell.
51. The kit of embodiment 49 or 50, further comprising one or more reagents for transforming the host cell with the one or plurality of vectors, one or more reagents for inducing expression of one or more cassettes within the one or plurality of vectors, or a combination thereof.
52. The kit of any one of embodiments 49 to 51, further comprising instructions for transforming the host cell, inducing expression of the one or more cassettes within the one or plurality of vectors, or a combination thereof.
53. A kit for modifying one or more target nucleic acids of interest at one or more target loci within a genome of a host cell, the kit comprising one or a plurality of gRNA-msr-msd-donor RNA molecules of any one of embodiments 24 to 30.
54. The kit of embodiment 53, further comprising a mammalian host cell.
55. The kit of embodiment 53 or 54, further comprising one or more reagents for introducing the one or plurality of gRNA-msr-msd-donor RNA molecules into the mammalian host cell.
56. The kit of embodiment 53, further comprising an RNA-guided nuclease-RT fusion protein or a plasmid for expressing an RNA-guided nuclease-RT fusion protein.
57. The kit of embodiment 53, further comprising instructions for introducing the one or plurality of gRNA-msr-msd-donor RNA molecules into the mammalian host cell, inducing expression of the RNA-guided nuclease-RT fusion protein, or a combination thereof.
58. The kit of embodiment 56 or 57, wherein the RNA-guided nuclease is saCas9, spCas9, or Cpf1.

### VII. Informal Sequence Listing

Certain sequences and constructs are referenced herein, which can have one or more of the following sequences, as applicable:

SEQ ID NO: 1
qPCR standard template-the single-stranded oligodeoxynucleotides (ssODNs)
sequence OBZ206:
GACGACGGCAACTACAAGACCCGCGCCGAGGTGAAGTTCGAGGGCGACACCCTGGTGAACCGCAT
CGAGCTGAAGGGCATCGACTTCAAGGAGGACGGCAACATCCTGGGGCACAAGCTGGAGTACAACT
ACAACAGCCACAACGTCTATATCACCGCCGACAAGCAGAAGAACGGCATCAAGGCCAACTTCAAG
ATCCG
SEQ ID NOS: 2-3
qPCR primers:
Forward primer: OBZ152 AGCTGAAGGGCATCGACTT (SEQ ID NO: 2)
Reverse primer: OBZ153 GGATCTTGAAGTTGGCCTTG (SEQ ID NO: 3)
SEQ ID NO: 4
Donor sequence (Donor sequence used in qPCR assay was amplified from the
TLR repair vector in Chu et al.):
TCTTCAAGGACGACGGCAACTACAAGACCCGCGCCGAGGTGAAGTTCGAGGGCGACACCCTGGTG
AACCGCATCGAGCTGAAGGGCATCGACTTCAAGGAGGACGGCAACATCCTGGGGCACAAGCTGGA
GTACAACTACAACAGCCACAACGTCTATATCACCGCCGACAAGCAGAAGAACGGCATCAAGGCCA
ACTTCAAGATCCG
SEQ ID NO: 5
Ec86 msr-msd sequence
ATGCGCACCCTTAGCGAGAGGTTTATCATTAAGGTCAACCTCTGGATGTTGTTTCGGCATCCTGCA
TTGAATCTGAGTTACTGTCTGTTTTCCTAGGAAACCCGTTTCTTCTGACGTAAGGGTGCGCA
SEQ ID NO: 6
Ec86 msr-msd sequence with “TTTT > TTTc” modification
ATGCGCACCCTTAGCGAGAGGTTTATCATTAAGGTCAACCTCTGGATGTTGTTTCGGCATCCTGCA
TTGAATCTGAGTTACTGTCTGTTTcCCTAGGAAACCCGTTTCTTCTGACGTAAGGGTGCGCA
SEQ ID NO: 7
Ec86 msr-msd sequence with “TTTT > TTTc” and “GGAAA > GGgAA” modifications
ATGCGCACCCTTAGCGAGAGGTTTATCATTAAGGTCAACCTCTGGATGTTGTTTCGGCATCCTGCA
TTGAATCTGAGTTACTGTCTGTTTcCCTAGGgAACCCGTTTCTTCTGACGTAAGGGTGCGCA
SEQ ID NO: 8
Ec86 msr-msd sequence with “TTTT > TTTc”, “GGAAA > GGgAA”, and “TTTTTT >
TTTcTT” modifications
ATGCGCACCCTTAGCGAGAGGTTTATCATTAAGGTCAACCTCTGGATGTTGTTTCGGCATCCTGCA
TTGAATCTGAGTTACTGTCTGTTTCCCTAGGgAACCCGTTTCTTCTGACGTAAGGGTGCGCA
SEQ ID NO: 9
Ec86 RT (amino acid sequence)
MKSAEYLNTFRLRNLGLPVMNNLHDMSKATRISVETLRLLIYTADFRYRIYTVEKKGPEKRMRTIYQPS
RELKALQGWVLRNILDKLSSSPFSIGFEKHQSILNNATPHIGANFILNIDLEDFFPSLTANKVFGVFHSLG
YNRLISSVLTKICCYKNLLPQGAPSSPKLANLICSKLDYRIQGYAGSRGLIYTRYADDLTLSAQSMKKVV
KARDFLFSIIPSEGLVINSKKTCISGPRSQRKVTGLVISQEKVGIGREKYKEIRAKIHHIFCGKSSEIEHVRG
WLSFILSVDSKSHRRLITYISKLEKKYGKNPLNKAKT
SEQ ID NO: 10
Ec107 msr-msd sequence
CGCCAGCAGTGGCAATAGCGTTTCCGGCCTTTTGTGCCGGGAGGGTCGGCGAGTCGCTGACTTAAC
GCCAGTAGTATGTCCATATACCCAAAGTCGCTTCATTGTACCTGAGTACGCTTCGCGTACGTCGCG
CTGACGCGCTCAGTACAGTTACGCGCCTTCGGGATGGTTTAATGGTATTGCCGCTGTTGGCG
SEQ ID NO: 11
Ec107 msr-msd sequence with “TTTT > TTTa” modification
CGCCAGCAGTGGCAATAGCGTTTCCGGCCTTTaGTGCCGGGAGGGTCGGCGAGTCGCTGACTTAAC
GCCAGTAGTATGTCCATATACCCAAAGTCGCTTCATTGTACCTGAGTACGCTTCGCGTACGTCGCG
CTGACGCGCTCAGTACAGTTACGCGCCTTCGGGATGGTTTAATGGTATTGCCGCTGTTGGOG
SEQ ID NO: 12
Ec107 RT (amino acid sequence)
MDATRTTLLALDLFGSPGWSADKEIQRLHALSNHAGRHYRRIILSKRHGGQRLVLAPDYLLKTVQRNI
LKNVLSQFPLSPFATAYRPGCPIVSNAQPHCQQPQILKLDIENFFDSISWLQVWRVFRQAQLPRNVVTM
LTWICCYNDALPQGAPTSPAISNLVMRRFDERIGEWCQARGITYTRYCDDMTFSGHFNARQVKNKVCG
LLAELGLSLNKRKGCLIAACKRQQVTGIVVNHKPQLAREARRALRQEVHLCQKYGVISHLSHRGELDP
SGDLHAQATAYLYALQGRINWLLQINPEDEAFQQARESVKRMLVAW
SEQ ID NO: 13
Mx65 msr-msd sequence
ggatccGGGCTCGCAGATGAGCCATGAGTACCGCGGTGTTTCGCCGCGGGGGTGTTCTGTCCCCATCT
CTTCGCCAGGGTCCCAGCGTACGCAACGCAGGGAGCCCCGGGTCCAACGCCTCGCAGGTCGTCCC
CTGGCCT
SEQ ID NO: 14
Mx65 RT (amino acid sequence)
MSWFDTTLSRLKGLFSRPVTRSTTGLDVPLDAHGRPQDVVTETVSTSGPLKPGHLRQVRRDARLLPKG
VRRYTPGRKKWMEAAEARRLFSATLRTRNRNLRDLLPDEAQLARYGLPVWRTEEDVAAALGVSVGV
LRHYSIHRPRERVRHYVTFAVPKRSGGVRLLHAPKRRLKALQRRMLALLVSKLPVSPQAHGFVPGRSI
KTGAAPHVGRRVVLKLDLKDFFPSVTFARVRGLLIALGYGYPVAATLAVLMTESERQPVELEGILFHVP
VGPRVCVQGAPTSPALCNAVLLRLDRRLAGLARRYGYTYTRYADDLTFSGDDVTALERVRALAARYV
QEEGFEVNREKTRVQRRGGAQRVTGVTVNTTLGLSREERPRLRAMLHQEARSEDVEAHRAHLDGLLA
YVKMLNPEQAERLARRRKPRGT
SEO ID NO: 15
Mx162 msr-msd sequence
GCGCGAGCAGCCGAGAGAGGTCCGGAGTGCATCAGCCTGAGCGCCTCGAGCGGCGGAGCGGCGTT
GCGCCGCTCCGGTTGGAATGCAGGACACTCTCCGCAAGGTAGCCTGTTCTTGGCTCTCTCCCTCCT
AGGCACTACGGCCAGGGTGGGTAGCGGAGCCAACGACGCGACCGCCGTTTACCCACCCCGGCCGT
AGTGCCTAGGAGGGGAGAGCCGGTGAGGCTACCGTGCCCC
SEO ID NO: 16
Mx162 RT (amino acid sequence)
MTARLDPFVPAASPQAVPTPELTAPSSDAAAKREARRLAHEALLVRAKAIDEAGGADDWVQAQLVSK
GLAVEDLDFSSASEKDKKAWKEKKKAEATERRALKRQAHEAWKATHVGHLGAGVHWAEDRLADAF
DVPHREERARANGLTELDSAEALAKALGLSVSKLRWFAFHREVDTATHYVSWTIPKRDGSKRTITSPK
PELKAAQRWVLSNVVERLPVHGAAHGFVAGRSILTNALAHQGADVVVKVDLKDFFPSVTWRRVKGL
LRKGGLREGTSTLLSLLSTEAPREAVQFRGKLLHVAKGPRALPQGAPTSPGITNALCLKLDKRLSALAK
RLGFTYTRYADDLTFSWTKAKQPKPRRTQRPPVAVLLSRVQEVVEAEGFRVHPDKTRVARKGTRQRV
TGLVVNAAGKDAPAARVPRDVVRQLRAAIHNRKKGKPGREGESLEQLKGMAAFIHMTDPAKGRAFL
AQLTELESTASAAPQAE
SEQ ID NO: 17
St85 msr-msd sequence
ACTCTTTAGCGTTAGGCTTTGATTTATAGCCTTGTCGAGCGTTTCGCCAGACACTAACTTATTGAGT
ACTTTTAGGGTTGCGCTAGAAAGTTTTCTACCGATCCTAGAAGTCTCTAGGATCGGTAGAAAACTT
TCTAGCGCCTCCTCTAGTAAAGAGT
SEO ID NO: 18
St85 msr-msd sequence with “TTTT > TTTc” modification
ACTCTTTAGCGTTAGGCTTTGATTTATAGCCTTGTCGAGCGTTTCGCCAGACACTAACTTATTGAGT
ACTTTCAGGGTTGCGCTAGAAAGTTTTCTACCGATCCTAGAAGTCTCTAGGATCGGTAGAAAACTTT
CTAGCGCCTCCTCTAGTAAAGAGT
SEO ID NO: 19
St85 msr-msd sequence with “TTTT > TTTc” and “GAAAA > GgAAA” modifications
ACTCTTTAGCGTTAGGCTTTGATTTATAGCCTTGTCGAGCGTTTCGCCAGACACTAACTTATTGAGT
ACTTTCAGGGTTGCGCTAGAAAGTTTTCTACCGATCCTAGAAGTCTCTAGGATCGGTAGgAAACTTT
CTAGCGCCTCCTCTAGTAAAGAGT
SEQ ID NO: 20
St85 RT (amino acid sequence)
MDILQHISDLLLTKKSEISFSLTAPYRYKIYKIAKRNSDKKRTIAHPSKELKFIQREITEYLTDKLPVHEC
AFAYKKGSSIKTNAQVHLHTKYLLKMDFENFFPSITPRLFFSKLRLANIDLTADDKVLLENILFFKSKRN
SNLRLSIGAPSSPLISNFVMYFWDIEVQEICSKIGVNYTRYADDLTFSTNNKDVLFDIPDMLENVLPKYSL
GRIRINHEKTVFSSKGHNRHVTGITLINDNKLSIGRERKRKISAMIHHFINGKLSTDECNKLVGLLAFAK
NIEPSFYKSMVIKYGSDNIYKLQKQKDK
SEQ ID NO: 21
Ec67 msr-msd sequence
CACGCATGTAGGCAGATTTGTTGGTTGTGAATCGCAACCAGTGGCCTTAATGGCAGGAGGAATCG
CCTCCCTAAAATCCTTGATTCAGAGCTATACGGCAGGTGTGCTGTGCGAAGGAGTGCCTGCATGCG
T
SEQ ID NO: 22
Ec67 RT (amino acid sequence)
MTKTSKLDALRAATSREDLAKILDIKLVFLTNVLYRIGSDNQYTQFTIPKKGKGVRTISAPTDRLKDIQR
RICDLLSDCRDEIFAIRKISNNYSFGFERGKSIILNAYKHRGKQIILNIDLKDFFESFNFGRVRGYFLSNQD
FLLNPVVATTLAKAACYNGTLPQGSPCSPIISNLICNIMDMRLAKLAKKYGCTYSRYADDITISTNKNTF
PLEMATVQPEGVVLGKVLVKEIENSGFEINDSKTRLTYKTSRQEVTGLTVNRIVNIDRCYYKKTRALAH
ALYRTGEYKVPDENGVLVSGGLDKLEGMFGFIDQVDKFNNIKKKLNKQPDRYVLTNATLHGFKLKLN
AREKAYSKFIYYKFFHGNTCPTIITEGKTDRIYLKAALHSLETSYPELFREKTDSKKKEINLNIFKSNEKT
KYFLDLSGGTADLKKFVERYKNNYASYYGSVPKQPVIMVLDNDTGPSDLLNFLRNKVKSCPDDVTEM
RKMKYIHVFYNLYIVLTPLSPSGEQTSMEDLFPKDILDIKIDGKKENKNNDGDSKTEYGKHIFSMRVVR
DKKRKIDFKAFCCIFDAIKDIKEHYKLMLNS
SEQ ID NO: 23
Sa163 msr-msd sequence
GTGCGAGCGACCGAGAGAGGTCCCAAGCCATCAGCCTCAGCGCCTCGAGCGCGAGAGCGGCGTTG
CGCCGCTCTGGTTGAATTGCAGGACACTCTCCGCAAGGTAGCCTGTTCTTGGCTCTCTTCCCTCCGG
TGAGTACCTCTCCGGCCGGGGAGCTGAACCAACGACGCAACCGCCGTTTCCCCGGCCGGAGAGGT
ACTCACCGGAGGGGAGAGCCGGTGAGGCTACCGTGCCCCAGGTGAGAAGGTGGTGCCTTCGGGCC
TCCCTCGACCGCTCGCGC
SEO ID NO: 24
Sa163 RT (amino acid sequence)
MTAKLESHVPAAPPVSAEAPAPTRPDAAKQEARRAHHEALRLRWKAIEEAGGTDAWVRQQLVAKGV
AAEEVDFESLSDKOKAAWKEKKKAEATERRAQKRLAWEAWKATHIHHLGVGVHWDEAGGPDKFDV
AGREERAKANGLPEGLDSVEALAKALGISVSRLRWFSFHREVDTGTHYQTWEIPKRDGGKRTLTAPKR
ELKAVQRWVLANVVERLPVHGAAHGFVAGRSILTNALAHQGADVVVKVDMKDFFPSVTWPRVKGLL
RKGGLPENLATLLALLSTEAPREVVRFRGETLYVAKGPRALPQGAPTSPALTNALCLRLDKRLSALSKR
LGFTYTRYADDLTFSWRRAKKSRQKELPLADAPVALLLARVKGVLEAEGFTLHPDKTRVQRKGSRQR
VTGLVVNEAPEGVPGARVPRDVVRRLRAAIHNREQGKPGPTGETLEQLKGLAAFLHMTDAEKGRAFL
RRLEALEKRQTA
SEQ ID NO: 25
Ec73 msr-msd sequence
AGAGCCAAACCTAGCATTTATGGGTTAATAGCCCATCGGGCCATGAGTCATGGTTTCGCCTAGTAT
TTTAGCTATGCCCGTCGTTATCGACGTGCTCAAGTAGGTTTGGCTCT
SEQ ID NO: 26
Ec73 RT (amino acid sequence)
MTKTSKLDALRAATSREDLAKILDIKLVFLTNVLYRIGSDNQYTQFTIPKKGKGVRTISAPTDRLKDIQR
RICDLLSDCRDEIFAIRKISNNYSFGFERGKSIILNAYKHRGKQIILNIDLKDFFESFNFGRVRGYFLSNQD
FLLNPVVATTLAKAACYNGTLPQGSPCSPIISNLICNIMDMRLAKLAKKYGCTYSRYADDITISTNKNTF
PLEMATVQPEGVVLGKVLVKEIENSGFEINDSKTRLTYKTSRQEVTGLTVNRIVNIDRCYYKKTRALAH
ALYRTGEYKVPDENGVLVSGGLDKLEGMFGFIDQVDKFNNIKKKLNKQPDRYVLTNATLHGFKLKLN
AREKAYSKFIYYKFFHGNTCPTIITEGKTDRIYLKAALHSLETSYPELFREKTDSKKKEINLNIFKSNEKT
KYFLDLSGGTADLKKFVERYKNNYASYYGSVPKQPVIMVLDNDTGPSDLLNFLRNKVKSCPDDVTEM
RKMKYIHVFYNLYIVLTPLSPSGEQTSMEDLFPKDILDIKIDGKKFNKNNDGDSKTEYGKHIFSMRVVR
DKKRKIDFKAFCCIFDAIKDIKEHYKLMLNS
Gene synthesis from Twist:
SEQ ID NO: 27
RT-EcoI/Ec86 in pTwist CMV:
ATGTACCCATACGACGTCCCAGACTACGCTCCTCCCAAGAAGAAAAGGAAAGTTAAGAGTGCGGA
ATACCTTAACACATTTAGGCTTAGAAACCTGGGACTCCCCGTTATGAATAACCTGCACGATATGTC
TAAGGCAACCCGAATCAGTGTAGAGACGCTTAGACTCTTGATATATACCGCCGACTTTCGATACAG
AATATATACCGTCGAGAAGAAGGGCCCAGAGAAACGAATGCGGACCATATACCAACCTAGTAGG
GAGCTGAAAGCGCTTCAGGGCTGGGTACTTCGAAATATCCTTGACAAACTTTCCAGTTCACCGTTC
AGCATTGGTTTCGAGAAACACCAGAGTATTCTGAATAACGCGACACCTCACATAGGAGCCAATTTC
ATCCTCAATATTGACCTGGAGGATTTCTTCCCTAGCCTTACTGCCAATAAAGTGTTCGGTGTATTCC
ACAGCCTCGGCTACAACCGACTGATTTCATCTGTACTCACAAAAATTTGTTGCTATAAGAACCTGC
TCCCTCAAGGGGCCCCAAGTAGCCCAAAACTGGCAAACCTCATCTGTTCAAAATTGGACTATCGAA
TTCAGGGCTATGCCGGATCCAGGGGCTTGATCTACACGAGATACGCAGACGACCTGACACTTTCAG
CACAATCCATGAAGAAAGTTGTTAAAGCGAGGGATTTTCTTTTTTCCATTATTCCGTCTGAAGGATT
GGTTATTAATTCTAAAAAAACTTGCATTAGTGGTCCTCGGTCTCAACGAAAGGTTACAGGCCTGGT
AATCTCTCAGGAGAAGGTTGGGATTGGAAGAGAGAAGTATAAAGAGATTCGCGCCAAAATACATC
ATATTTTTTGCGGTAAATCATCTGAAATCGAGCACGTAAGGGGATGGCTTTCTTTCATTCTTTCTGT
AGACTCCAAATCTCACCGACGACTCATCACATATATAAGTAAACTGGAGAAAAAATATGGGAAAA
ATCCGCTTAACAAGGCTAAAACTAAAAGGCCGGCGGCCACGAAAAAGGCCGGCCAGGCAAAAAA
GAAAAAGGACTATAAAGATGACGATGACAAGGACTACAAGGATGATGACGATAAAGATTATAAA
GACGACGATGATAAGAAAGGTCCGGGAAGCGGAGCTACTAACTTCAGCCTGCTGAAGCAGGCTGG
AGACGTGGAGGAGAACCCTGGACCTCCGGTCGCCACCAGCGAGCTGATTAAGGAGAACATGCACA
TGAAGCTGTACATGGAGGGCACCGTGGACAACCATCACTTCAAGTGCACATCCGAGGGCGAAGGC
AAGCCCTACGAGGGCACCCAGACCATGAGAATCAAGGTGGTCGAGGGCGGCCCTCTCCCCTTCGC
CTTCGACATCCTGGCTACTAGCTTCCTCTACGGCAGCAAGACCTTCATCAACCACACCCAGGGCAT
CCCCGACTTCTTCAAGCAGTCCTTCCCTGAGGGCTTCACATGGGAGAGAGTCACCACATACGAAGA
CGGGGGCGTGCTGACCGCTACCCAGGACACCAGCCTCCAGGACGGCTGCCTCATCTACAACGTCA
AGATCAGAGGGGTGAACTTCACATCCAACGGCCCTGTGATGCAGAAGAAAACACTCGGCTGGGAG
GCCTTCACCGAGACGCTGTACCCCGCTGACGGCGGCCTGGAAGGCAGAAACGACATGGCCCTGAA
GCTCGTGGGCGGGAGCCATCTGATCGCAAACATCAAGACCACATATAGATCCAAGAAACCCGCTA
AGAACCTCAAGATGCCTGGCGTCTACTATGTGGACTACAGACTGGAAAGAATCAAGGAGGCCAAC
AACGAGACCTACGTCGAGCAGCACGAGGTGGCAGTGGCCAGATACTGCGACCTCCCTAGCAAACT
GGGGCACAAGCTTAATTAATTAAGAATTCGTCGAGGGACCTAATAACTTCGTATAGCATACATTAT
ACGAAGTTATACATGTTTAAGGGTTCCGGTTCCACTAGGTACAATTCGATATCAAGCTTATCGATA
ATCAACCTCTGGATTACAAAATTTGTGAAAGATTGACTGGTATTCTTAACTATGTTGCTCCTTTTAC
GCTATGTGGATACGCTGCTTTAATGCCTTTGTATCATGCTATTGCTTCCCGTATGGCTTTCATTTTCT
CCTCCTTGTATAAATCCIGGTTGCTGTCTCTTTATGAGGAGTTGTGGCCCGTTGTCAGGCAACGTGG
CGTGGTGTGCACTGTGTTTGCTGACGCAACCCCCACTGGTTGGGGCATTGCCACCACCTGTCAGCT
CCTTTCCGGGACTTTCGCTTTCCCCCTCCCTATTGCCACGGCGGAACTCATCGCCGCCTGCCTTGCC
CGCTGCTGGACAGGGGCTCGGCTGTTGGGCACTGACAATTCCGTGGTGTTGTCGGGGAAATCATCG
TCCTTTCCTTGGCTGCTCGCCTGTGTTGCCACCTGGATTCTGCGCGGGACGTCCTTCTGCTACGTCC
CTTCGGCCCTCAATCCAGCGGACCTTCCTTCCCGCGGCCTGCTGCCGGCTCTGCGGCCTCTTCCGCG
TCTTCGCCTTCGCCCTCAGACGAGTCGGATCTCCCTTTGGGCCGCCTCCCCGCAGCGGCCGCGAGG
TGCTTGTAGATAACCTCCACGATGGTGCACCTTGGGCAACACAAAAGTGGCAAATCATCTACAATG
CGCACCCTTAGCGAGAGGTTTATCATTAAGGTCAACCTCTGGATGTTGTTTCGGCATCCTGCATTG
AATCTGAGTTACTGTCTGTTTTCCTGGCCATGCTGGCCAGCGGATAACAAGTTTAAACACGGCCAC
AGTGGCCAGGAAACCCGTTTCTTCTGACGTAAGGGTGCGCAGACACTGAGTGAGAAACGTCCCCG
TCGTAGTGTCGGTAATGCGTTGTTTCAACGTAGCCAATTCTCACGGCGCGCC
SEQ ID NO: 28
RT-EcoII/Ec67 in pTwist EF1 Alpha Puro:
ATGTACCCATACGACGTCCCAGACTACGCTCCTCCCAAGAAGAAAAGGAAAGTTATGACTAAAAC
CTCTAAACTCGACGCACTCAGAGCAGCAACATCCCGCGAAGACCTCGCCAAAATTTTGGATATTAA
ACTTGTGTTCCTTACAAATGTCCTGTATCGCATAGGGTCAGATAATCAATACACACAGTTTACGAT
CCCGAAGAAGGGTAAGGGCGTGCGAACCATATCAGCCCCGACCGACCGGCTGAAGGACATTCAGC
GGAGAATCTGTGATTTGTTGAGCGACTGTCGGGATGAAATCTTTGCGATTCGGAAGATAAGTAATA
ATTATTCTTTCGGATTTGAAAGGGGGAAATCCATAATTCTCAATGCCTATAAACACCGCGGTAAGC
AGATTATCCTTAACATTGATCTTAAAGATTTCTTTGAGTCCTTCAACTTCGGTAGGGTACGAGGTTA
CTTTCTTTCTAACCAGGATTTTTTGCTCAATCCTGTTGTGGCGACTACACTGGCAAAAGCGGCATGC
TATAATGGGACCCTGCCTCAGGGCAGCCCTTGCAGCCCAATAATATCAAACCTTATCTGCAACATC
ATGGATATGCGCTTGGCAAAGCTGGCCAAGAAATACGGATGTACATACAGTCGGTACGCCGATGA
TATCACCATCTCTACAAATAAGAATACTTTTCCTCTCGAAATGGCCACAGTTCAACCGGAGGGCGT
CGTCCTGGGGAAGGTGCTCGTAAAAGAAATCGAAAACTCTGGTTTCGAGATCAATGATTCTAAGA
CGAGACTGACTTATAAAACTAGTAGACAGGAGGTTACGGGGCTGACCGTAAACCGCATAGTAAAT
ATCGATCGATGCTACTATAAGAAAACAAGGGCGCTTGCACACGCACTTTATCGGACAGGTGAATA
TAAGGTCCCTGACGAGAACGGCGTCCTGGTTTCAGGCGGGCTGGATAAGCTCGAAGGAATGTTTG
GCTTTATCGATCAAGTCGACAAATTCAACAATATAAAGAAAAAGCTTAACAAACAGCCAGATAGG
TACGTCTTGACAAACGCCACACTGCATGGGTTCAAGCTGAAACTCAACGCTCGCGAGAAGGCGTA
CTCAAAGTTCATTTACTACAAGTTTTTCCATGGTAATACGTGTCCTACTATCATAACTGAGGGGAA
GACAGACCGCATATATCTGAAGGCAGCTCTCCACAGTCTCGAGACCTCATATCCCGAACTTTTTCG
CGAGAAGACGGATTCAAAAAAGAAGGAAATAAATTTGAATATCTTCAAATCAAACGAAAAGACA
AAATATTTCCTCGACCTCTCTGGCGGGACCGCGGATCTTAAGAAATTCGTAGAGCGGTACAAGAAT
AATTACGCGTCCTATTATGGATCCGTTCCCAAGCAGCCAGTCATAATGGTTCTCGATAACGACACT
GGGCCGAGCGATCTCCTCAATTTTCTGAGAAACAAAGTGAAATCCTGTCCTGATGATGTGACTGAA
ATGCGCAAAATGAAGTATATACACGTCTTCTACAATCTCTACATCGTGCTGACGCCATTGAGCCCC
TCAGGGGAGCAAACGTCAATGGAGGACCTCTTCCCCAAGGATATTCTCGACATCAAGATAGATGG
AAAAAAGTTCAACAAAAACAATGATGGGGATTCAAAGACGGAATATGGAAAACACATCTTCTCAA
TGCGAGTCGTACGCGATAAAAAAAGGAAGATCGACTTCAAAGCTTTTTGTTGCATTTTCGACGCAA
TCAAGGATATAAAAGAACATTATAAGCTTATGTTGAACAGCAAAAGGCCGGCGGCCACGAAAAAG
GCCGGCCAGGCAAAAAAGAAAAAGGACTATAAAGATGACGATGACAAGGACTACAAGGATGATG
ACGATAAAGATTATAAAGACGACGATGATAAG
Twist gene fragments:
SEQ ID NO: 29
RT-EcoIII/Ec73
GAAGTGCCATTCCGCCTGACCTATGTACCCATACGACGTCCCAGACTACGCTCCTCCCAAGAAGAA
AAGGAAAGTTATGCGAATATACTCCCTTATAGACAGTCAGACCCTCATGACCAAGGGGTTTGCCTC
AGAGGTCATGCGGTCACCAGAACCCCCGAAGAAGTGGGACATAGCAAAAAAGAAGGGTGGTATG
CGGACAATTTATCACCCATCATCTAAAGTTAAACTTATCCAGTACTGGCTCATGAACAACGTATTT
AGTAAGCTGCCAATGCACAACGCGGCTTATGCTTTTGTTAAGAATCGGTCAATCAAGTCTAACGCG
CTTCTCCACGCAGAGTCAAAAAACAAATACTACGTTAAGATCGATCTCAAAGACTTCTTTCCCTCA
ATTAAGTTCACGGATTTTGAGTACGCTTTCACTAGGTATCGCGACAGAATCGAATTTACAACCGAG
TATGACAAAGAATTGCTGCAGTTGATCAAGACTATCTGTTTTATCTCTGACAGCACGCTCCCGATA
GGTTTCCCAACCAGCCCACTCATTGCGAATTTCGTGGCGAGGGAGCTGGACGAGAAACTTACCCA
GAAACTCAATGCTATAGATAAACTTAATGCGACATATACCCGCTATGCAGACGATATTATTGTCTC
AACTAATATGAAAGGAGCGAGCAAATTGATCCTTGACTGCTTCAAGCGAACTATGAAGGAGATCG
GGCCGGACTTCAAGATCAATATCAAAAAGTTCAAAATATGTAGCGCCAGCGGGGGGTCAATAGTG
GTCACGGGGCTGAAAGTGTGCCACGATTTTCACATTACGCTTCATCGGAGTATGAAAGATAAAATA
AGGTTGCACTTGAGCTTGCTCTCTAAGGGAATTTTGAAAGACGAAGACCATAACAAACTTTCAGGT
TATATTGCATACGCTAAAGACATAGACCCCCACTTTTATACCAAACTTAACCGGAAATACTTCCAA
GAAATTAAGTGGATCCAAAACCTGCATAATAAAGTTGAAAAAAGGCCGGCGGCCACGAAAAAGG
CCGGCCAGGCAAAAAAGAAAAAGGACTATAAAGATGACGATGACAAGGACTACAAGGATGATGA
CGATAAAGATTATAAAGACGACGATGATAAGAGGCTAGGTGGAGGCTCAGTG
SEQ ID NO: 30
RT-EcoV/Ec107
GAAGTGCCATTCCGCCTGACCTATGTACCCATACGACGTCCCAGACTACGCTCCTOCCAAGAAGAA
AAGGAAAGTTATGGACGCCACCCGAACCACTCTTCTTGCGTTGGATCTCTTCGGGTCACCAGGGTG
GTCCGCCGACAAGGAGATACAGCGCCTTCACGCTCTGAGCAATCATGCTGGCCGCCATTATAGGA
GGATCATTCTTTCTAAACGGCACGGAGGCCAGAGACTCGTCTTGGCGCCGGATTACTTGTTGAAAA
CTGTCCAACGGAATATACTGAAAAACGTATTGTCCCAATTTCCGTTGTCACCCTTTGCCACTGCCTA
TCGACCAGGATGCCCTATCGTATCTAACGCGCAACCACACTGCCAGCAACCACAGATACTGAAATT
GGACATTGAAAATTTCTTTGACTCTATCAGTTGGCTCCAAGTATGGCGCGTTTTCAGACAGGCTCA
GCTCCCTCGAAATGTTGTGACAATGCTTACCTGGATTTGTTGTTACAACGATGCCCTCCCGCAAGG
TGCGCCAACATCCCCTGCGATATCTAATCTCGTCATGAGGCGGTTCGATGAGAGAATCGGGGAATG
GTGCCAGGCTCGCGGAATCACATACACGCGGTATTGTGACGACATGACATTTTCCGGCCATTTCAA
CGCCCGACAAGTAAAGAACAAAGTGTGTGGCCTTCTCGCAGAGCTTGGGTTGTCTCTGAATAAGC
GAAAAGGGTGCCTTATTGCGGCTTGTAAGCGGCAGCAAGTGACAGGTATTGTGGTGAACCATAAA
CCGCAACTTGCAAGAGAGGCCCGCCGGGCTCTCCGACAGGAGGTGCATCTCTGTCAGAAATATGG
GGTCATCTCACACTTGAGTCACAGGGGTGAACTCGACCCGAGTGGAGACCTGCACGCCCAGGCGA
CGGCCTACTTGTACGCGCTTCAAGGAAGGATAAACTGGCTCCTTCAAATCAATCCTGAAGACGAA
GCGTTCCAGCAGGCCAGGGAGTCTGTTAAACGGATGCTCGTAGCATGGAAAAGGCCGGCGGCCAC
GAAAAAGGCCGGCCAGGCAAAAAAGAAAAAGGACTATAAAGATGACGATGACAAGGACTACAAG
GATGATGACGATAAAGATTATAAAGACGACGATGATAAGAGGCTAGGTGGAGGCTCAGTG
SEO ID NO: 31
RT-MaxII/Mx65
GAAGTGCCATTCCGCCTGACCTATGTACCCATACGACGTCCCAGACTACGCTCCTOCCAAGAAGAA
AAGGAAAGTTATGAGTTGGTTCGATACAACTCTGTCTCGGTTGAAAGGGCTTTTTTCAAGGCCGGT
TACTCGGAGCACAACAGGACTGGATGTCCCATTGGACGCGCACGGGAGGCCGCAGGACGTTGTTA
CAGAAACTGTCAGTACCAGTGGTCCTCTCAAGCCAGGTCATCTTCGCCAAGTGAGACGAGATGCTC
GGCTTCTCCCGAAAGGTGTTCGACGCTACACCCCCGGCCGAAAGAAATGGATGGAGGCCGCGGAG
GCCAGGCGACTCTTTTCTGCCACGCTGCGAACGAGAAACCGAAATCTGAGAGATCTGTTGCCAGA
CGAAGCACAACTTGCTCGCTATGGTTTGCCAGTCTGGCGCACCGAAGAAGACGTGGCAGCGGCCC
TCGGAGTATCAGTTGGCGTCCTTAGGCATTACTCTATACATAGGCCGCGCGAGCGGGTGAGGCACT
ACGTAACGTTCGCGGTCCCAAAACGAAGCGGTGGCGTCAGGTTGCTCCATGCACCTAAACGGCGG
CTTAAAGCACTCCAGCGCCGGATGCTGGCACTTCTGGTAAGCAAACTCCCTGTGTCCCCACAGGCT
CATGGTTTCGTGCCCGGAAGAAGTATAAAGACTGGGGCGGCCCCTCATGTAGGTAGGCGCGTAGT
CCTCAAGCTCGATTTGAAGGACTTCTTTCCGTCTGTTACGTTTGCGCGGGTTAGAGGCCTGCTTATT
GCACTCGGCTATGGTTATCCTGTTGCTGCTACACTCGCAGTCCTTATGACGGAATCAGAGCGCCAA
CCTGTCGAGTTGGAAGGAATCCTGTTTCATGTCCCAGTTGGCCCAAGAGTTTGCGTACAGGGGGCG
CCTACTTCCCCAGCACTTTGCAACGCTGTTCTTCTTCGATTGGACAGGAGACTTGCCGGCCTTGCAA
GACGATATGGTTACACGTACACACGGTATGCGGACGACTTGACATTCTCAGGAGACGACGTCACG
GCGTTGGAGCGAGTTAGAGCTCTTGCGGCAAGATATGTCCAAGAAGAAGGTTTCGAGGTGAACAG
AGAGAAAACTCGCGTGCAGAGGCGAGGCGGAGCCCAGCGAGTGACGGGCGTGACCGTGAACACG
ACACTGGGACTGTCAAGGGAAGAACGCCCTAGGTTGCGAGCAATGCTCCACCAGGAGGCACGATC
TGAAGATGTGGAAGCACACCGAGCCCATCTGGATGGCTTGTTGGCCTACGTCAAAATGCTGAACC
CCGAACAAGCCGAACGGTTGGCCCGACGCCGCAAGCCGCGGGGGACCAAAAGGCCGGCGGCCAC
GAAAAAGGCCGGCCAGGCAAAAAAGAAAAAGGACTATAAAGATGACGATGACAAGGACTACAAG
GATGATGACGATAAAGATTATAAAGACGACGATGATAAGAGGCTAGGTGGAGGCTCAGTG
SEQ ID NO: 32
RT-Mxa1/Mx162
GAAGTGCCATTCCGCCTGACCTATGTACCCATACGACGTCCCAGACTACGCTCCTCCCAAGAAGAA
AAGGAAAGTTATGACAGCAAGGTTGGACCCCTTTGTCCCAGCAGCTTCACCCCAGGCAGTGCCAA
CCCCAGAACTGACGGCGCCCAGCTCCGATGCGGCTGCAAAACGAGAGGCACGGCGCCTGGCGCAT
GAAGCGTTGTTGGTAAGGGCGAAAGCTATCGATGAAGCTGGGGGTGCCGACGATTGGGTTCAGGC
CCAACTGGTGTCAAAAGGATTGGCGGTGGAAGATCTCGACTTCTCTTCTGCCTCAGAGAAAGATAA
AAAGGCATGGAAGGAAAAGAAGAAAGCGGAGGCAACAGAAAGGCGGGCCCTTAAGAGGCAAGC
CCATGAGGCGTGGAAAGCAACCCATGTTGGACACTTGGGCGCGGGAGTCCACTGGGCCGAAGACA
GACTCGCGGATGCCTTCGACGTACCGCATAGGGAAGAACGGGCACGGGCGAATGGGCTGACCGAG
CTGGACAGTGCGGAAGCGTTGGCAAAAGCCCTTGGCTTGTCAGTGTCAAAACTCCGATGGTTTGCA
TTCCATAGAGAGGTAGATACAGCCACTCATTATGTATCTTGGACGATCCCAAAGAGGGACGGCTCC
AAGCGAACGATAACATCCCCCAAGCCCGAGTTGAAAGCCGCTCAAAGGTGGGTTCTCTCAAATGT
GGTGGAAAGACTCCCGGTCCATGGTGCTGCTCACGGGTTTGTCGCTGGTCGCAGCATACTCACTAA
CGCTCTTGCTCATCAGGGAGCGGATGTAGTCGTAAAAGTTGACCTTAAAGACTTTTTTCCTAGTGT
GACCTGGCGCAGGGTAAAGGGCCTCTTGAGGAAAGGTGGACTCAGAGAGGGCACAAGTACGTTGC
TTAGTCTTCTTTCTACCGAAGCACCTCGCGAGGCTGTCCAATTTCGAGGCAAATTGCTCCATGTTGC
AAAGGGTCCACGGGCGCTTCCACAGGGAGCCCCTACCTCACCGGGGATCACCAACGCACTCTGTC
TCAAGCTTGATAAGCGCCTTTCAGCCCTCGCCAAGAGGTTGGGTTTTACTTACACGCGCTACGCGG
ACGATTTGACCTTTTCTTGGACTAAGGCGAAGCAGCCAAAGCCACGACGGACACAACGGCCTCCA
GTCGCGGTTCTCTTGTCTCGCGTCCAAGAAGTGGTTGAGGCCGAAGGCTTTAGGGTCCATCCTGAC
AAGACCCGAGTTGCCCGGAAGGGAACGCGACAACGCGTTACGGGGTTGGTGGTAAATGCGGCGG
GGAAAGACGCCCCAGCCGCTCGGGTCCCCCGCGACGTGGTAAGGCAGCTTAGGGCCGCGATTCAT
AACCGAAAGAAGGGCAAGCCCGGACGGGAAGGGGAGTCTCTGGAACAATTGAAGGGCATGGCTG
CATTTATTCACATGACAGACCCCGCAAAAGGTCGGGCATTTCTGGCACAGCTTACGGAGCTGGAA
AGTACTGCGTCCGCCGCACCTCAAGCAGAAAAAAGGCCGGCGGCCACGAAAAAGGCCGGCCAGG
CAAAAAAGAAAAAGGACTATAAAGATGACGATGACAAGGACTACAAGGATGATGACGATAAAGA
TTATAAAGACGACGATGATAAGAGGCTAGGTGGAGGCTCAGTG
SEQ ID NO: 33
RT-SenII/S185
GAAGTGCCATTCCGCCTGACCTATGTACCCATACGACGTCCCAGACTACGCTCCTOCCAAGAAGAA
AAGGAAAGTTATGGATATTCTGCAACACATATCCGACCTGCTGCTGACAAAGAAGTCAGAAATTA
TTTCTTTTAGTCTCACCGCACCTTATCGGTACAAAATTTATAAGATCGCTAAAAGAAACTCCGACA
AGAAAAGAACCATCGCCCACCCGAGTAAAGAACTTAAATTCATACAGAGAGAGATTACTGAGTAC
CTGACAGATAAGCTGCCCGTGCACGAATGCGCCTTTGCTTACAAAAAGGGCTCTAGTATAAAAAC
AAACGCACAAGTTCATCTCCATACGAAATATCTGTTGAAAATGGATTTTGAAAACTTTTTTCCCTCC
ATAACGCCGCGGCTCTTCTTCTCAAAACTGCGCTTGGCTAACATCGACTTGACTGCTGATGATAAG
GTTTTGTTGGAGAATATCTTGTTTTTTAAATCCAAACGCAACAGTAATCTGCGCTTGAGTATCGGTG
CGCCGTCTTCTCCATTGATTTCCAACTTCGTTATGTACTTCTGGGACATTGAGGTGCAAGAGATATG
CTCCAAGATCGGGGTTAATTATACCCGCTACGCAGATGATTTGACCTTTAGCACAAATAATAAAGA
CGTGCTGTTTGATATACCTGATATGTTGGAAAACGTTCTGCCGAAGTATAGTTTGGGGCGGATACG
GATTAACCACGAAAAGACTGTGTTCTCTAGCAAGGGCCACAACCGACATGTAACCGGGATAACTC
TTACTAATGATAACAAGCTGTCTATCGGTCGGGAGCGGAAAAGGAAGATCAGCGCGATGATCCAC
CACTTCATCAATGGAAAGCTGTCAACAGATGAGTGCAACAAATTGGTGGGCCTCCTGGCCTTCGCC
AAAAATATAGAGCCATCCTTTTACAAAAGCATGGTTATTAAGTATGGGAGTGATAACATTTATAAA
CTCCAGAAGCAGAAAGACAAGAAAAGGCCGGCGGCCACGAAAAAGGCCGGCCAGGCAAAAAAG
AAAAAGGACTATAAAGATGACGATGACAAGGACTACAAGGATGATGACGATAAAGATTATAAAG
ACGACGATGATAAGAGGCTAGGTGGAGGCTCAGTG
gBlock Gene Fragments ordered from IDT:
SEQ ID NO: 34
RT-SauI_Sal63
ATGTACCCATACGACGTCCCAGACTACGCTCCTCCCAAGAAGAAAAGGAAAGTTATGACGGCAAA
ACTGGAAAGCCACGTGCCTGCTGCACCACCAGTCTCAGCGGAAGCGCCGGCACCGACGCGACCTG
ACGCTGCTAAGCAGGAGGCTCGACGAGCCCACCATGAAGCCCTGAGACTGCGGTGGAAAGCAATC
GAGGAAGCTGGGGGCACTGACGCCTGGGTACGACAGCAGTTGGTTGCGAAAGGCGTAGCGGCGG
AAGAGGTGGACTTCGAAAGTCTCTCCGACAAGCAAAAAGCGGCTTGGAAAGAGAAAAAAAAAGC
TGAGGCAACCGAGCGGAGGGCTCAAAAACGACTTGCCTGGGAAGCCTGGAAAGCGACCCATATTC
ATCATTTGGGCGTTGGAGTTCACTGGGACGAAGCCGGGGGCCTGACAAGTTCGATGTGGCAGGG
AGGGAAGAAAGGGCAAAGGCAAACGGGTTGCCCGAAGGTCTTGACAGCGTAGAAGCATTGGCGA
AAGCGCTGGGAATCTCAGTATCCCGATTGCGCTGGTTCTCTTTTCACAGGGAAGTTGATACAGGGA
CACACTACCAAACTTGGGAGATCCCAAAGAGGGATGGGGGCAAACGCACCCTCACGGCCCCGAAA
AGGGAGCTTAAGGCAGTACAAAGGTGGGTACTCGCCAATGTGGTGGAAAGACTTCCCGTACACGG
AGCTGCTCATGGGTTTGTTGCAGGACGCTCCATACTCACTAATGCGCTCGCTCACCAAGGAGCTGA
CGTGGTTGTTAAAGTTGATATGAAAGATTTTTTTOCCAGTGTCACATGGCCTCGAGTTAAAGGCCT
GCTCAGAAAGGGAGGTTTGCCTGAAAACTTGGCGACCCTGCTCGCCCTTCTGAGTACCGAAGCTCC
ACGGGAAGTAGTCCGATTCAGAGGGGAAACGCTGTACGTCGCTAAGGGCCCACGCGCTCTGCCAC
AGGGCGCGCCTACCAGTCCTGCGCTTACTAACGCATTGTGTCTTAGGTTGGATAAGCGGTTGAGCG
CGCTGAGTAAGCGGTTGGGTTTCACATACACCAGATACGCTGACGATCTTACATTTAGTTGGAGGC
GCGCTAAAAAATCTCGCCAGAAAGAGTTGCCGCTGGCTGATGCTCCCGTCGCGCTTCTCCTGGCCC
GGGTTAAGGGAGTGTTGGAAGCAGAAGGTTTCACTTTGCATCCTGATAAGACCAGGGTTCAGCGG
AAGGGAAGCCGGCAACGAGTGACCGGCCTCGTGGTTAATGAAGCGCCCGAAGGAGTGCCAGGTG
CCCGAGTTCCCCGAGATGTTGTTCGGAGATTGCGAGCGGCAATACACAATAGAGAGCAAGGTAAA
CCAGGGCCGACTGGAGAAACGTTGGAACAACTCAAAGGCTTGGCAGCTTTTCTGCACATGACAGA
CGCTGAAAAAGGAAGAGCGTTTCTGAGAAGACTTGAGGCCCTGGAGAAGAGGCAAACTGCGAAA
AGGCCGGCGGCCACGAAAAAGGCCGGCCAGGCAAAAAAGAAAAAGGACTATAAAGATGACGATG
ACAAGGACTACAAGGATGATGACGATAAAGATTATAAAGACGACGATGATAAG
Plasmids used in FACS plots
SEQ ID NO: 35
pBZ186-pU6-(BbsI)_CBh-sv40NLS-Cas9-NILS TZA-mCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccgggtcttcgagaagacctgttttagagctagaaatagcaagttaaaataaggctagtccgttatcaacttgaaaaagtggcaccga
gtcggtgcttttttgttttagagctagaaatagcaagttaaaataaggctagtccgttttlagcgcgtgcgccaattctgcagacaaatggctctagaggtacccttac
ataacttacggtaaatggcccgcctggctgaccgcccaacgacccccgcccattgacgtcaatagtaacgccaatagggactttccattgacgtcaatgggtggag
tatttacggtaaactgcccacttggcagtacatcaagtgtatcatatgccaagtacgccccctattgacgtcaatgacggtaaatggcccgcctggcattgtgcccagt
acatgaccttatgggactttcctacttggcagtacatctacgtattagtcatcgctattaccatggtcgaggtgagccccacgttctgcttcactctccccatctcccccc
ctccccacccccaattttgtatttatttattttttaattattttgtgcagcgatgggggcggggggggggggggggcgcgcgccaggcggggcggggcggggcg
aggggggggcggggcgaggcggagaggtgcggcggcagccaatcagagcggcgcgctccgaaagtttccttttatggcgaggcggcggcggcggcggc
cctataaaaagcgaagcgcgcggggggggagtcgctgcgacgctgccttcgccccgtgccccgctccgccgccgcctcgcgccgcccgccccggctctg
actgaccgcgttactcccacaggtgagcggggggacggcccttctcctccgggctgtaattagctgagcaagaggtaagggtttaagggatggttggttggtgg
ggtattaatgtttaattacctggagcacctgcctgaaatcactttttttcaggttggaccggtgccaccatggactataaggaccacgacggagactacaaggatcatg
atattgattacaaagacgatgacgataagatggccccaaagaagaagcggaaggtcggtatccacggagtcccagcagccgacaagaagtacagcatcggcct

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

aaaaggccggcggccacgaaaaaggccggccaggcaaaaaagaaaaagcttgaggg
cagaggaagtctgctaacatgcggtgacgtggaggagaatcccggccctgcta

[symbol]

[symbol]

[symbol]

[symbol]

[symbol]

[symbol]

[symbol]

[symbol]

agcctcgactgtgccttctagttgccagccatctgttgtttgcccctcccccgtgccttccttgaccctggaaggtgccactcccactgtcctttcctaataaaatgagg
aaattgcatcgcattgtctgagtaggtgtcattctattctggggggtggggggggcaggacagcaagggggaggattgggaagagaatagcaggcatgctggg
gagcggccgcaggaacccctagtgatggagttggccactccctctctgcgcgctcgctcgctcactgaggccgggcgaccaaaggtcgcccgacgcccgggc
tttgcccgggcggcctcagtgagcgagcgagcgcgcagctgcctgcaggggcgcctgatgcggtattttctccttacgcatctgtgcggtatttcacaccgcatac
gtcaaagcaaccatagtacgcgccctgtagcggcgcattaagcgcggcgggtgtggtggttacgcgcagcgtgaccgctacacttgccagcgccctagcgccc
gctcctttcgctttcttcccttcctttctcgccacgttcgccggctttccccgtcaagctctaaatcgggggctccctttagggttccgatttagtgctttacggcacctcg
accccaaaaaacttgatttgggtgatggttcacgtagtgggccatcgccctgatagacggtttttcgccctttgacgttggagtccacgttctttaatagtggactcttgt
tccaaactggaacaacactcaaccctatctcgggctattcttttgatttataagggattttgccgatttcggCctattggttaaaaaatgagctgatttaacaaaaatttaa
cgcgaattttaacaaaatattaacgtttacaattttatggtgcactctcagtacaatctgctctgatgccgcatagttaagccagccccgacacccgccaacacccgct
gacgcgccctgacgggcttgtctgctcccggcatccgcttacagacaagctgtgaccgtctccgggagctgcatgtgtcagaggttttcaccgtcatcaccgaaac
gcgcgagacgaaagggcctcgtgatacgcctatttttataggttaatgtcatgataataatggtttcttagacgtcaggtggcacttttcggggaaatgtgcgcggaac
ccctatttgtttatttttctaaatacattcaaatatgtatccgctcatgagacaataaccctgataaatgcttcaataatattgaaaaaggaagagtatgagtattcaacattt
ccgtgtcgcccttattcccttttttgcggcattttgccttcctgtttttgctcacccagaaacgctggtgaaagtaaaagatgctgaagatcagttgggtgcacgagtggg
ttacatcgaactggatctcaacagcggtaagatccttgagagttttcgccccgaagaacgttttccaatgatgagcacttttaaagttctgctatgtggcgcggtattatc
ccgtattgacgccgggcaagagcaactcggtcgccgcatacactattctcagaatgacttggttgagtactcaccagtcacagaaaagcatcttacggatggcatg
acagtaagagaattatgcagtgctgccataaccatgagtgataacactgcggccaacttacttctgacaacgatcggaggaccgaaggagctaaccgcttttttgca
caacatgggggatcatgtaactcgccttgatcgttgggaaccggagctgaatgaagccataccaaacgacgagcgtgacaccacgatgcctgtagcaatggcaa
caacgttgcgcaaactattaactggcgaactacttactctagcttcccggcaacaattaatagactggatggaggcggataaagttgcaggaccacttctgcgctcg
gcccttccggctggctggtttattgctgataaatctggagccggtgagcgtggaagccgcggtatcattgcagcactggggccagatggtaagccctcccgtatcg
tagttatctacacgacggggagtcaggcaactatggatgaacgaaatagacagatcgctgagataggtgcctcactgattaagcattggtaactgtcagaccaagtt
tactcatatatactttagattgatttaaaacttcatttttaatttaaaaggatctaggtgaagatcctttttgataatctcatgaccaaaatcccttaacgtgagttttcgttccac
tgagcgtcagaccccgtagaaaagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgtccttctagtgtagccgtagttaggccaccactt
caagaactctgtagcaccgcctacatacctcgctctgctaatcctgttaccagtggctgctgccagtggcgataagtcgtgtcttaccgggttggactcaagacgata
gttaccggataaggcgcagcggtcgggctgaacggggggttcgtgcacacagcccagcttggagcgaacgacctacaccgaactgagatacctacagcgtga
gctatgagaaagcgccacgcttcccgaagggagaaaggcggacaggtatccggtaagcggcagggtcggaacaggagagcgcacgagggagcttccaggg
ggaaacgcctggtatctttatagtcctglcgggtttcgccacctctgacttgagcgtcgatttttgtgatgctcgtcaggggggcggagcctatggaaaaacgccagc
aacgcggcctttttacggttcctggccttttgctggccttttgctcacatgt
SEQ ID NO: 36
pBZ191-pU6-sgBFP-CBh-sv40NLS-Cas9-NLS-T2A-mCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcttttttgttttagagctagaaatagcaagttaaaataaggctagtccgtttttagcgcgtgcgccaattctgcagacaaatggctctaga
ggtacccgttacataacttacggtaaatggcccgcctggctgaccgcccaacgacccccgcccattgacgtcaatagtaacgccaatagggactttccattgacgt
caatgggtggagtatttacggtaaactgcccacttggcagtacatcaagtgtatcatatgccaagtacgccccctattgacgtcaatgacggtaaatggcccgcctg
gcattgtgcccagtacatgaccttatgggactttcctacttggcagtacatctacgtattagtcatcgctattaccatggtcgaggtgagccccacgttctgcttcactct
ccccatctcccccccctccccacccccaattttgtatttatttattttttaattattttgtgcagcgatgggggcggggggggggggggggcgcgcgccaggcgggg
cggggcggggcgaggggcggggcggggcgaggcggagaggtgcggcggcagccaatcagagcggcgcgctccgaaagtttccttttatggcgaggcggc
ggcggcggcggccctataaaaagcgaagcgcgcggcgggcgggagtcgctgcgacgctgccttcgccccgtgccccgctccgccgccgcctcgcgccgcc
cgccccggctctgactgaccgcgttactcccacaggtgagcggggggacggcccttctcctccgggctgtaattagctgagcaagaggtaagggtttaagggat
ggttggttggtggggtattaatgtttaattacctggagcacctgcctgaaatcactttttttcaggttggaccggtgccaccatggactataaggaccacgacggagac
tacaaggatcatgatattgattacaaagacgatgacgataagatggccccaaagaagaagcggaaggtcggtatccacggagtcccagcagccgacaagaagta

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

aaaaggccggcggccacgaaaaaggccggccaggcaaaaaagaaaa
agcttgaggcagaggaagtctgctaacatgcggtgacgtggaggagaatcccggccctgctagc

[symbol]

[symbol]

[symbol]

[symbol]

[symbol]

[symbol]

[symbol]

[symbol]

gctcgctgatcagcctcgactgtgccttctagttgccagccatctgttgtttgcccctcccccgtgccttccttgaccctggaaggtgccactcccactgtcctttcctaa
taaaatgaggaaattgcatcgcattgtctgagtaggtgtcattctattctggggggggggtggggcaggacagcaagggggaggattgggaagagaatagcag
gcatgctggggagcggccgcaggaacccctagtgatggagttggccactccctctctgcgcgctcgctcgctcactgaggccgggcgaccaaaggtcgcccga
cgcccgggctttgcccgggcggcctcagtgagcgagcgagcgcgcagctgcctgcaggggcgcctgatgcggtattttctccttacgcatctgtgcggtatttcac
accgcatacgtcaaagcaaccatagtacgcgccctgtagcggcgcattaagcgcggcgggtgtggtggttacgcgcagcgtgaccgctacacttgccagcgcc
ctagcgcccgctcctttcgctttcttcccttcctttctcgccacgttcgccggctttccccgtcaagctctaaatcgggggctccctttagggttccgatttagtgctttac
ggcacctcgaccccaaaaaacttgatttgggtgatggttcacgtagtgggccatcgccctgatagacggtttttcgccctttgacgttggagtccacgttctttaatagt
ggactcttgttccaaactggaacaacactcaaccctatctcgggctattcttttgatttataagggattttgccgatttcggCctattggttaaaaaatgagctgatttaac
aaaaatttaacgcgaattttaacaaaatattaacgtttacaattttatggtgcactctcagtacaatctgctctgatgccgcatagttaagccagccccgacacccgcca
acacccgctgacgcgccctgacgggcttgtctgctcccggcatccgcttacagacaagctgtgaccgtctccgggagctgcatgtgtcagaggttttcaccgtcat
caccgaaacgcgcgagacgaaagggcctcgtgatacgcctatttttataggttaatgtcatgataataatggtttcttagacgtcaggtggcacttttcggggaaatgt
gcgcggaacccctatttgtttatttttctaaatacattcaaatatgtatccgctcatgagacaataaccctgataaatgcttcaataatattgaaaaaggaagagtatgagt
attcaacatttccgtgtcgcccttattcccttttttgcggcattttgccttcctgtttttgctcacccagaaacgctggtgaaagtaaaagatgctgaagatcagttgggtgc
acgagtgggttacatcgaactggatctcaacagcggtaagatccttgagagttttcgccccgaagaacgttttccaatgatgagcacttttaaagttctgctatgtggc
gcggtattatcccgtattgacgccgggcaagagcaactcggtcgccgcatacactattctcagaatgacttggttgagtactcaccagtcacagaaaagcatcttac
ggatggcatgacagtaagagaattatgcagtgctgccataaccatgagtgataacactgcggccaacttacttctgacaacgatcggaggaccgaaggagctaac
cgcttttttgcacaacatgggggatcatgtaactcgccttgatcgttgggaaccggagctgaatgaagccataccaaacgacgagcgtgacaccacgatgcctgta
gcaatggcaacaacgttgcgcaaactattaactggcgaactacttactctagcttcccggcaacaattaatagactggatggaggcggataaagttgcaggaccact
tctgcgctcggcccttccggctggctggtttattgctgataaatctggagccggtgagcgtggaagccgcggtatcattgcagcactggggccagatggtaagccc
tcccgtatcgtagttatctacacgacggggagtcaggcaactatggatgaacgaaatagacagatcgctgagataggtgcctcactgattaagcattggtaactgtc
agaccaagtttactcatatatactttagattgatttaaaacttcatttttaatttaaaaggatctaggtgaagatcctttttgataatctcatgaccaaaatcccttaacgtgag
ttttcgttccactgagcgtcagaccccgtagaaaagatcaaaggatcttcttgagatcctttttttctgcgcgtaatctgctgcttgcaaaaaaaaaaccaccgctacc
agcggtggtttgtttgccggatcaagagctaccaactctttttccgaaggtaactggcttcagcagagcgcagataccaaatactgtccttctagtgtagccgtagtta
ggccaccacttcaagaactctgtagcaccgcctacatacctcgctctgctaatcctgttaccagtggctgctgccagtggcgataagtcgtgtcttaccgggttggac
tcaagacgatagttaccggataaggcgcagcggtcgggctgaacggggggttcgtgcacacagcccagcttggagcgaacgacctacaccgaactgagatacc
tacagcgtgagctatgagaaagcgccacgcttcccgaagggagaaaggcggacaggtatccggtaagcggcagggtcggaacaggagagcgcacgaggga
gcttccagggggaaacgcctggtatctttatagtcctgtcgggtttcgccacctctgacttgagcgtcgatttttgtgatgctcgtcaggggggcggagcctatggaa
aaacgccagcaacgcggcctttttacggttcctggccttttgctggccttttgct
SEQ ID NO: 37
pBZ193-pU6-sgBFP20- [symbol] -CBh-sv40NLS-Cas9-NLS-T2A-mCherry-P2A- [symbol]
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaaCACCGCTGAAGCACTGCACGCCATgttttagagctagaaatagcaagttaaaataaggctagtccgttatcaacttga
aaaagtggcaccgagtcggtgcCGTACGGTGCGAGCGACCGAGAGAGGTCCCAAGCCATCAGCCTCAGCGCCT
CGAGCGCGAGAGCGGCGTTGCGCCGCTCTGGTTGAATTGCAGGACACTCTCCGCAAC=GGTAGCCTG

[symbol]

[symbol]

[symbol]

CCTAGGCGCAACCGCCGTTTCCCCGGCCGGAGAGGTACTCACCGGAGGGGAGAGCCGGTGAGGCT
ACCGTGCCCCAGGTGAGAAGGTGGTGCCTTCGGGCCTCCCTCGACCGCTCGCGCtttttttctagaggtacccgtt
acataacttacggtaaatggcccgcctggctgaccgcccaacgacccccgcccattgacgtcaatagtaacgccaatagggactttccattgacgtcaatgggtgg
agtatttacggtaaactgcccacttggcagtacatcaagtgtatcatatgccaagtacgccccctattgacgtcaatgacggtaaatggcccgcctggcattgtgccc
agtacatgaccttatgggactttcctacttggcagtacatctacgtattagtcatcgctattaccatggtcgaggtgagccccacgttctgcttcactctcccctctccc
ccccctccccacccccaattttgtatttatttattttttaattattttgtgcagcgatgggggcggggggggggggggggcgcgcgccaggcggggcggggcggg
gcgaggggcggggcggggcgaggcggagaggtgcggcggcagccaatcagagcggcgcgctccgaaagtttccttttatggcgaggcggcggcggcggc
ggccctataaaaagcgaagcgcgcggcgggcgggagtcgtcgcgacgctgccttcgccccgtgccccgctccgccgccgcctcgcgccgcccgccccggct
ctgactgaccgcgttactcccacaggtgagcgggcgggacggcccttctcctccgggctgtaattagctgagcaagaggtaagggtttaagggatggttggttggt
ggggtattaatgtttaattacctggagcacctgcctgaaatcactttttttcaggttggaccggtgccaccatggactataaggaccacgacggagactacaaggatc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

tgccttccttgaccctggaaggtgccactcccactgtcctttcctaataaaatgaggaaattgcatcgcattgtctgagtaggtgtcattctattctggggggtggggtg
gggcaggacagcaagggggaggattgggaagagaatagcaggcatgctggggagcggccgcaggaacccctagtgatggagttggccactccctctctgcg
cgctcgctcgctcactgaggccgggcgaccaaaggtcgcccgacgcccgggctttgcccgggcggcctcagtgagcgagcgagcgcgcagctgcctgcagg
ggcgcctgatgcggtattttctccttacgcatctgtgcggtatttcacaccgcatacgtcaaagcaaccatagtacgcgccctgtagcggcgcattaagcgcggcgg
gtgtggtggttacgcgcagcgtgaccgctacacttgccagcgccctagcgcccgctcctttcgctttcttcccttcctttctcgccacgttcgccggctttccccgtcaa
gctctaaatcgggggctccctttagggttccgatttagtgctttacggcacctcgaccccaaaaaacttgatttgggtgatggttcacgtagtgggccatcgccctgat
agacggtttttcgccctttgacgttggagtccacgttctttaatagtggactcttgttccaaactggaacaacactcaaccctatctcgggctattcttttgatttataaggg
attttgccgatttcggcctattggttaaaaaatgagctgatttaacaaaaatttaacgcgaattttaacaaaatattaacgtttacaattttatggtgcactctcagtacaatc
tgctctgatgccgcatagttaagccagccccgacacccgccaacacccgctgacgcgccctgacgggcttgtctgctcccggcatccgcttacagacaagctgtg
accgtctccgggagctgcatgtgtcagaggttttcaccgtcatcaccgaaacgcgcgagacgaaagggcctcgtgatacgcctatttttataggttaatgtcatgata
ataatggtttcttagacgtcaggtggcacttttcggggaaatgtgcgcggaacccctatttgtttatttttctaaatacattcaaatatgtatccgctcatgagacaataacc
ctgataaatgcttcaataatattgaaaaaggaagagtatgagtattcaacatttccgtgtcgcccttattcccttttttgcggcattttgccttcctgtttttgctcacccaga
aacgctggtgaaagtaaaagatgctgaagatcagttgggtgcacgagtgggttacatcgaactggatctcaacagcggtaagatccttgagagttttcgccccgaa
gaacgttttccaatgatgagcacttttaaagttctgctatgtggcgcggtattatcccgtattgacgccgggcaagagcaactcggtcgccgcatacactattctcaga
atgacttggttgagtactcaccagtcacagaaaagcatcttacggatggcatgacagtaagagaattatgcagtgctgccataaccatgagtgataacactgcggcc
aacttacttctgacaacgatcggaggaccgaaggagctaaccgcttttttgcacaacatgggggatcatgtaactcgccttgatcgttgggaaccggagctgaatga
agccataccaaacgacgagcgtgacaccacgatgcctgtagcaatggcaacaacgttgcgcaaactattaactggcgaactacttactctagcttcccggcaacaa
ttaatagactggatggaggcggataaagttgcaggaccacttctgcgctcggcccttccggctggctggtttattgctgataaatctggagccggtgagcgtggaag
ccgcggtatcattgcagcactggggccagatggtaagccctcccgtatcgtagttatctacacgacggggagtcaggcaactatggatgaacgaaatagacagat
cgctgagataggtgcctcactgattaagcattggtaactgtcagaccaagtttactcatatatactttagattgatttaaaacttcatttttaatttaaaaggatctaggtgaa
gatcctttttgataatctcatgaccaaaatcccttaacgtgagttttcgttccactgagcgtcagaccccgtagaaaagatcaaaggatcttcttgagatcctttttttctgc
gcgtaatctgctgcttgcaaacaaaaaaaccaccgctaccagcggtggtttgtttgccggatcaagagctaccaactctttttccgaaggtaactggcttcagcagag
cgcagataccaaatactgtccttctagtgtagccgtagttaggccaccacttcaagaactctgtagcaccgcctacatacctcgctctgctaatcctgttaccagtggc
tgctgccagtggcgataagtcgtgtcttaccgggttggactcaagacgatagttaccggataaggcgcagcggtcgggctgaacggggggttcgtgcacacagc
ccagcttggagcgaacgacctacaccgaactgagatacctacagcgtgagctatgagaaagcgccacgcttcccgaagggagaaaggcggacaggtatccgg
taagcggcagggtcggaacaggagagcgcacgagggagcttccagggggaaacgcctggtatctttatagtcctgtcgggtttcgccacctctgacttgagcgtc
gatttttgtgatgctcgtcaggggggcggagcctatggaaaaacgccagcaacgcggcctttttacggttcctggccttttgctggccttttgctcacatgt
SEQ ID NO: 38
pBZ194-pU6-sgBFP- [symbol] -CBh-sv40NLS-Cas9-NLS-T2A-mCherry-P2A- [symbol]
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaaCACCGCTGAAGCACTGCACGCCATgttttagagctagaaatagcaagttaaaataaggctagtccgttatcaacttga
aaaagtggcaccgagtcggtgcCGTACGGTGCGAGCGACCGAGAGAGGTCCCAAGCCATCAGCCTCAGCGCCT
CGAGCGCGAGAGCGGCGTTGCGCCGCTCTGGTTGAATTGCAGGACACTCTCCGCAAGGTAGCCTG
TTCTTGGCTCTCTTCCCTCCGGTGAGTACCTCTCCGGCCGGGGAGCTGAACCAACGAACTAGTICA

[symbol]

[symbol]

[symbol]

CTACCGTGCCCCAGGTGAGAAGGTGGTGCCTTCGGGCCTCCCTCGACCGCTCGCGCtttttttctagaggtacc
cgttacataacttacggtaaatggcccgcctggctgaccgcccaacgacccccgcccattgacgtcaatagtaacgccaatagggactttccattgacgtcaatgg
gtggagtatttacggtaaactgcccacttggcagtacatcaagtgtatcatatgccaagtacgccccctattgacgtcaatgacggtaaatggcccgcctggcattgt
gcccagtacatgaccttatgggactttcctacttggcagtacatctacgtattagtcatcgctattaccatggtcgaggtgagccccacgttctgcttcactctccccatc
tcccccccctccccacccccaattttgtatttatttattttttaattattttgtgcagcgatggggggggggggggggggggggggccaggcggggggggc
ggggcgaggggggggggggcgaggcggagaggtgcggcggcagccaatcagagcggcgcgctccgaaagtttccttttatggcgaggcggcggcggc
ggcggccctataaaaagcgaagcgcgcggcgggcgggagtcgctgcgacgctgccttcgccccgtgccccgctccgccgccgcctcgcgccgcccgcccc
ggctctgactgaccgcgttactcccacaggtgagcggggggacggcccttctcctccgggctgtaattagctgagcaagaggtaagggtttaagggatggttgg
ttggggggtattaatgtttaattacctggagcacctgcctgaaatcactttttttcaggttggaccggtgccaccatggactataaggaccacgacggagactacaag

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ccgtgccttccttgaccctggaaggtgccactcccactgtcctttcctaataaaatgaggaaattgcatcgcattgtctgagtaggtgtcattctattctggggggtggg
gtggggcaggacagcaagggggaggattgggaagagaatagcaggcatgctggggagcggccgcaggaacccctagtgatggagttggccactccctctct
gcgcgctcgctcgctcactgaggccgggcgaccaaaggtcgcccgacgcccgggctttgcccgggcggcctcagtgagcgagcgagcgcgcagctgcctgc
aggggcgcctgatgcggtattttctccttacgcatctgtgcggtatttcacaccgcatacgtcaaagcaaccatagtacgcgccctgtagcggcgcattaagcgcgg
cgggtgtggtggttacgcgcagcgtgaccgctacacttgccagcgccctagcgcccgctcctttcgctttcttcccttcctttctcgccacgttcgccggctttccccg
tcaagctctaaatcgggggctccctttagggttccgatttagtgctttacggcacctcgaccccaaaaaacttgatttgggtgatggttcacgtagtgggccatcgccc
tgatagacggtttttcgccctttgacgttggagtccacgttctttaatagtggactcttgttccaaactggaacaacactcaaccctatctcgggctattcttttgatttataa
gggattttgccgatttcggcctattggttaaaaaatgagctgatttaacaaaaatttaacgcgaattttaacaaaatattaacgtttacaattttatggtgcactctcagtac
aatctgctctgatgccgcatagttaagccagccccgacacccgccaacacccgctgacgcgccctgacgggcttgtctgctcccggcatccgcttacagacaagc
tgtgaccgtctccgggagctgcatgtgtcagaggttttcaccgtcatcaccgaaacgcgcgagacgaaagggcctcgtgatacgcctatttttataggttaatgtcat
gataataatggtttcttagacgtcaggtggcacttttcggggaaatgtgcgcggaacccctatttgtttatttttctaaatacattcaaatatgtatccgctcatgagacaat
aaccctgataaatgcttcaataatattgaaaaaggaagagtatgagtattcaacatttccgtgtcgcccttattcccttttttgcggcattttgccttcctgtttttgctcaccc
agaaacgctggtgaaagtaaaagatgctgaagatcagttgggtgcacgagtgggttacatcgaactggatctcaacagcggtaagatccttgagagttttcgcccc
gaagaacgttttccaatgatgagcacttttaaagttctgctatgtggcgcggtattatcccgtattgacgccgggcaagagcaactcggtcgccgcatacactattctc
agaatgacttggttgagtactcaccagtcacagaaaagcatcttacggatggcatgacagtaagagaattatgcagtgctgccataaccatgagtgataacactgcg
gccaacttacttctgacaacgatcggaggaccgaaggagctaaccgcttttttgcacaacatgggggatcatgtaactcgccttgatcgttgggaaccggagctga
atgaagccataccaaacgacgagcgtgacaccacgatgcctgtagcaatggcaacaacgttgcgcaaactattaactggcgaactacttactctagcttcccggca
acaattaatagactggatggaggcggataaagttgcaggaccacttctgcgctcggcccttccggctggctggtttattgctgataaatctggagccggtgagcgtg
gaagccgcggtatcattgcagcactggggccagatggtaagccccccgtatcgtagttatctacacgacggggagtcaggcaactatggatgaacgaaatagac
agatcgctgagataggtgcctcactgattaagcattggtaactgtcagaccaagtttactcatatatactttagattgatttaaaacttcatttttaatttaaaaggatctagg
tgaagatcctttttgataatctcatgaccaaaatcccttaacgtgagttttcgttccactgagcgtcagaccccgtagaaaagatcaaaggatcttcttgagatccttttttt
ctgcgcgtaatctgctgcttgcaaacaaaaaaaccaccgctaccagcggtggtttgtttgccggatcaagagctaccaactctttttccgaaggtaactggcttcagc
agagcgcagataccaaatactgtccttctagtgtagccgtagttaggccaccacttcaagaactctgtagcaccgcctacatacctcgctctgctaatcctgttaccag
tggctgctgccagtggcgataagtcgtgtcttaccgggttggactcaagacgatagttaccggataaggcgcagcggtcgggctgaacggggggttcgtgcaca
cagcccagcttggagcgaacgacctacaccgaactgagatacctacagcgtgagctatgagaaagcgccacgcttcccgaagggagaaaggcggacaggtat
ccggtaagcggcagggtcggaacaggagagcgcacgagggagcttccagggggaaacgcctggtatctttatagtcctgtcgggtttcgccacctctgacttga
gcgtcgatttttgtgatgctcgtcaggggggcggagcctatggaaaaacgccagcaacgcggcctttttacggttcctggccttttgctggccttttgctcacatgt
SEQ ID NO: 39
pBZ195-pU6-sgBFP- [symbol] -CBh-sv40NLS-Cas9-NLS-T2A-mCherry-P2A- [symbol]
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaaCACCGCTGAAGCACTGCACGCCATgttttagagctagaaatagcaagttaaaataaggctagtccgttatcaacttga
aaaagtggcaccgagtcggtgcCGTACGGTGCGAGCGACCGAGAGAGGTCCCAAGCCATCAGCCTCAGCGCCT
CGAGCGCGAGAGCGGCGTTGCGCCGCTCTGGTTGAATTGCAGGACACTCTCCGCAAGGTAGCCTG

[symbol]

[symbol]

[symbol]

GCAACCGCCGTTTCCCCGGCCGGAGAGGTACTCACCGGAGGGGAGAGCCGGTGAGGCTACCGTGC
CCCAGGTGAGAAGGTGGTGCCTTCGGGCCTCCCTCGACCGCTCGCGCtttttttctagaggtacccgttacataacttacg
gtaaatggcccgcctggctgaccgcccaacgacccccgcccattgacgtcaatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattgtgcccagtacatgacct
tatgggactttcctacttggcagtacatctacgtattagtcatcgctattaccatggtcgaggtgagccccacgttctgcttcactctccccatctcccccccctccccac
ccccaattttgtatttatttattttttaattattttgtgcagcgatgggggcggggggggggggggggcgcgcgccaggcggggggggggggcgagggggg
ggcggggcgaggcggagaggtgcggcggcagccaatcagagcggcgcgctccgaaagtttccttttatggcgaggcggcggcggcggcggccctataaaaa
gcgaagcgcgcggcggggggagtcgctgcgacgctgccttcgccccgtgccccgctccgccgccgcctcgcgccgcccgccccggctctgactgaccgcg
ttactcccacaggtgagcggggggacggcccttctcctccgggctgtaattagctgagcaagaggtaagggtttaagggatggttggttggtggggtattaatgtt
taattacctggagcacctgcctgaaatcactttttttcaggttggaccggtgccaccatggactataaggaccacgacggagactacaaggatcatgatattgattaca

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gaaggtgccactcccactgtcctttcctaataaaatgaggaaattgcatcgcattgtctgagtaggtgtcattctattctggggggggggtggggcaggacagcaa
gggggaggattgggaagagaatagcaggcatgctggggagcggccgcaggaacccctagtgatggagttggccactccctctctgcgcgctcgctcgctcact
gaggccgggcgaccaaaggtcgcccgacgcccgggctttgcccgggcggcctcagtgagcgagcgagcgcgcagctgcctgcaggggcgcctgatgcggt
attttctccttacgcatctgtgcggtatttcacaccgcatacgtcaaagcaaccatagtacgcgccctgtagcggcgcattaagcgcggcgggtgtggtggttacgc
gcagcgtgaccgctacacttgccagcgccctagcgcccgctccttcgctttcttcccttcctttctcgccacgttcgccggctttccccgtcaagctctaaatcgggg
gctccctttagggttccgatttagtgctttacggcacctcgaccccaaaaaacttgatttgggtgatggttcacgtagtgggccatcgccctgatagacggtttttcgcc
ctttgacgttggagtccacgttctttaatagtggactcttgttccaaactggaacaacactcaaccctatctcgggctattcttttgatttataagggattttgccgatttcgg
cctattggttaaaaaatgagctgatttaacaaaaatttaacgcgaattttaacaaaatattaacgtttacaattttatggtgcactctcagtacaatctgctctgatgccgca
tagttaagccagccccgacacccgccaacacccgctgacgcgccctgacgggcttgtctgctcccggcatccgcttacagacaagctgtgaccgtctccgggag
ctgcatgtgtcagaggttttcaccgtcatcaccgaaacgcgcgagacgaaagggcctcgtgatacgcctatttttataggttaatgtcatgataataatggtttcttaga
cgtcaggtggcacttttcggggaaatgtgcgcggaaccccfatttgtttatttttctaaatacattcaaatatgtatccgctcatgagacaataaccctgataaatgcttca
ataatattgaaaaaggaagagtatgagtattcaacatttccgtgtcgcccttattcccttttttgcggcattttgccttcctgtttttgctcacccagaaacgctggtgaaag
taaaagatgctgaagatcagttgggtgcacgagtgggttacatcgaactggatctcaacagcggtaagatccttgagagttttcgccccgaagaacgttttccaatga
tgagcacttttaaagttctgctatgtggcgcggtattatcccgtattgacgccgggcaagagcaactcggtcgccgcatacactattctcagaatgacttggttgagta
ctcaccagtcacagaaaagcatcttacggatggcatgacagtaagagaattatgcagtgctgccataaccatgagtgataacactgcggccaacttacttctgacaa
cgatcggaggaccgaaggagctaaccgcttttttgcacaacatgggggatcatgtaactcgccttgatcgttgggaaccggagctgaatgaagccataccaaacg
acgagcgtgacaccacgatgcctgtagcaatggcaacaacgttgcgcaaactattaactggcgaactacttactctagcttcccggcaacaattaatagactggatg
gaggcggataaagttgcaggaccacttctgcgctcggcccttccggctggctggtttattgctgataaatctggagccggtgagcgtggaagccgcggtatcattg
cagcactggggccagatggtaagccctcccgtatcgtagttatctacacgacggggagtcaggcaactatggatgaacgaaatagacagatcgctgagataggtg
cctcactgattaagcattggtaactgtcagaccaagtttactcatatatactttagattgatttaaaacttcatttttaatttaaaaggatctaggtgaagatcctttttgataat
ctcatgaccaaaatcccttaacgtgagttttcgttccactgagcgtcagaccccgtagaaaagatcaaaggatcttcttgagatcctttttttctgcgcgtaatctgctgct
tgcaaacaaaaaaaccaccgctaccagcggtggtttgtttgccggatcaagagctaccaactctttttccgaaggtaactggcttcagcagagcgcagataccaaat
actgtccttctagtgtagccgtagttaggccaccacttcaagaactctgtagcaccgcctacatacctcgctctgctaatcctgttaccagtggctgctgccagtggcg
ataagtcgtgtcttaccgggttggactcaagacgatagttaccggataaggcgcagcggtcgggctgaacggggggttcgtgcacacagcccagcttggagcga
acgacctacaccgaactgagatacctacagcgtgagctatgagaaagcgccacgcttcccgaagggagaaaggcggacaggtatccggtaagcggcagggtc
ggaacaggagagcgcacgagggagcttccagggggaaacgcctggtatctttatagtcctgtcgggtttcgccacctctgacttgagcgtcgatttttgtgatgctc
gtcaggggggcggagcctatggaaaaacgccagcaacgcggcctttttacggttcctggccttttgctggccttttgctcacatgt
SEQ ID NO: 40
pBZ196-pU6-sgBFP- [symbol] -CBh-sv40NLS-Cas9-NLS-T2A-Cherry-P2A- [symbol]
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaaCACCGCTGAAGCACTGCACGCCATgttttagagctagaaatagcaagttaaaataaggctagtccgttatcaacttga
aaaagtggcaccgagtcggtgcCGTACGGTGCGAGCGACCGAGAGAGGTCCCAAGCCATCAGCCTCAGCGCCT
CGAGCGCGAGAGCGGCGTTGCGCCGCTCTGGTTGAATTGCAGGACACTCTCCGCAAGGTAGCCTG

[symbol]

[symbol]

GCGCAACCGCCGTTTCCCCGGCCGGAGAGGTACTCACCGGAGGGGAGAGCCGGTGAGGCTACCGT
GCCCCAGGTGAGAAGGTGGTGCCTTCGGGCCTCCCTCGACCGCTCGCGCtttttttctagaggtacccgttacataactt
acggtaaatggcccgcctggctgaccgcccaacgacccccgcccattgacgtcaatagtaacgccaatagggactttccattgacgtcaatgggtggagtatttac
ggtaaactgcccacttggcagtacatcaagtgtatcatatgccaagtacgccccctattgacgtcaatgacggtaaatggcccgcctggcattgtgcccagtacatg
accttatgggactttcctacttggcagtacatctacgtattagtcatcgctattaccatggtcgaggtgagccccacgttctgcttcactctccccatctcccccccctcc
ccacccccaattttgtatttatttattttttaattattttgtgcagcgatgggggcggggggggggggggggcgcgcgccaggcggggggggggggcgaggg
gcggggggggcgaggcggagaggtgcggcggcagccaatcagagcggcgcgctccgaaagtttccttttatggcgaggcggcggcggcggcggccctat
aaaaagcgaagcgcgcggggggggagtcgctgcgacgctgccttcgccccgtgccccgctccgccgccgcctcgcgccgcccgccccggctctgactga
ccgcgttactcccacaggtgagcggggggacggcccttctcctccgggctgtaattagctgagcaagaggtaagggtttaagggatggttggttggtggggtatt
aatgtttaattacctggagcacctgcctgaaatcactttttttcaggttggaccggtgccaccatggactataaggaccacgacggagactacaaggatcatgatattg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gaccctggaaggtgccactcccactgtcctttcctaataaaatgaggaaattgcatcgcattgtctgagtaggtgtcattctattctggggggtggggggggcagga
cagcaagggggaggattgggaagagaatagcaggcatgctggggagcggccgcaggaacccctagtgatggagttggccactccctctctgcgcgctcgctc
gctcactgaggccgggcgaccaaaggtcgcccgacgcccgggctttgcccgggcggcctcagtgagcgagcgagcgcgcagctgcctgcaggggcgcctg
atgcggtattttctccttacgcatctgtgcggtatttcacaccgcatacgtcaaagcaaccatagtacgcgccctgtagcggcgcattaagcgcggcgggtgtggtg
gttacgcgcagcgtgaccgctacacttgccagcgccctagcgcccgctcctttcgctttcttcccttcctttctcgccacgttcgccggctttccccgtcaagctctaaa
tcgggggctccctttagggttccgatttagtgctttacggcacctcgaccccaaaaaacttgatttgggtgatggttcacgtagtgggccatcgccctgatagacggtt
tttcgccctttgacgttggagtccacgttctttaatagtggactcttgttccaaactggaacaacactcaaccctatctcgggctattcttttgatttataagggattttgccg
atttcggcctattggttaaaaaatgagctgatttaacaaaaatttaacgcgaattttaacaaaatattaacgtttacaattttatggtgcactctcagtacaatctgctctgat
gccgcatagttaagccagccccgacacccgccaacacccgctgacgcgccctgacgggcttgtctgctcccggcatccgcttacagacaagctgtgaccgtctc
cgggagctgcatgtgtcagaggttttcaccgtcatcaccgaaacgcgcgagacgaaagggcctcgtgatacgcctatttttataggttaatgtcatgataataatggtt
tcttagacgtcaggtggcacttttcggggaaatgtgcgcggaacccctatttgtttatttttctaaatacattcaaatatgtatccgctcatgagacaataaccctgataaa
tgcttcaataatattgaaaaaggaagagtatgagtattcaacatttccgtgtcgcccttattcccttttttgcggcattttgccttcctgtttttgctcacccagaaacgctgg
tgaaagtaaaagatgctgaagatcagttgggtgcacgagtgggttacatcgaactggatctcaacagcggtaagatccttgagagttttcgccccgaagaacgtttt
ccaatgatgagcacttttaaagttctgctatgtggcgcggtattatcccgtattgacgccgggcaagagcaactcggtcgccgcatacactattctcagaatgacttgg
ttgagtactcaccagtcacagaaaagcatcttacggatggcatgacagtaagagaattatgcagtgctgccataaccatgagtgataacactgcggccaacttacttc
tgacaacgatcggaggaccgaaggagctaaccgcttttttgcacaacatgggggatcatgtaactcgccttgatcgttgggaaccggagctgaatgaagccatacc
aaacgacgagcgtgacaccacgatgcctgtagcaatggcaacaacgttgcgcaaactattaactggcgaactacttactctagcttcccggcaacaattaatagact
ggatggaggggataaagttgcaggaccacttctgcgctcggcccttccggctggctggtttattgctgataaatctggagccggtgagcgtggaagccgcggtat
cattgcagcactggggccagatggtaagccctcccgtatcgtagttatctacacgacggggagtcaggcaactatggatgaacgaaatagacagatcgctgagat
aggtgcctcactgattaagcattggtaactgtcagaccaagtttactcatatatactttagattgatttaaaacttcatttttaatttaaaaggatctaggtgaagatccttttt
gataatctcatgaccaaaatcccttaacgtgagttttcgttccactgagcgtcagaccccgtagaaaagatcaaaggatcttcttgagatcctttttttctgcgcgtaatct
gctgcttgcaaacaaaaaaaccaccgctaccagcggtggtttgtttgccggatcaagagctaccaactctttttccgaaggtaactggcttcagcagagcgcagata
ccaaatactgtccttctagtgtagccgtagttaggccaccacttcaagaactctgtagcaccgcctacatacctcgctctgctaatcctgttaccagtggctgctgcca
gtggcgataagtcgtgtcttaccgggttggactcaagacgatagttaccggataaggcgcagcggtcgggctgaacggggggttcgtgcacacagcccagcttg
gagcgaacgacctacaccgaactgagatacctacagcgtgagctatgagaaagcgccacgcttcccgaagggagaaaggcggacaggtatccggtaagcggc
agggtcggaacaggagagcgcacgagggagcttccagggggaaacgcctggtatctttatagtcctgtcgggtttcgccacctctgacttgagcgtcgatttttgtg
atgctcgtcaggggggcggagcctatggaaaaacgccagcaacgcggcctttttacggttcctggccttttgctggccttttgctcacatgt
SEQ ID NO: 41
pBZ197-pU6-sgBFP- [symbol] -CBh-sv40NLS-Cas9-NLS-T2A-mCherry-P2A- [symbol]
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaaCACCGCTGAAGCACTGCACGCCATgttttagagctagaaatagcaagttaaaataaggctagtccgttatcaacttga
aaaagtggcaccgagtcggtgcCGTACGGTGCGAGCGACCGAGAGAGGTCCCAAGCCATCAGCCTCAGCGCCT
CGAGCGCGAGAGCGGCGTTGCGCCGCTCTGGTTGAATTGCAGGACACTCTCCGCAAGGTAGCCTG

[symbol]

[symbol]

[symbol]

CCGTTTCCCCGGCCGGAGAGGTACTCACCGGAGGGGAGAGCCGGTGAGGCTACCGTGCCCCAGGT
GAGAAGGTGGTGCCTTCGGGCCTCCCTCGACCGCTCGCGCtttttttctagaggtacccgttacataacttacggtaaatggccc
gcctggctgaccgcccaacgacccccgcccattgacgtcaatagtaacgccaatagggactttccattgacgtcaatgggtggagtatttacggtaaactgcccact
tggcagtacatcaagtgtatcatatgccaagtacgccccctattgacgtcaatgacggtaaatggcccgcctggcattgtgcccagtacatgaccttatgggactttc
ctacttggcagtacatctacgtattagtcatcgctattaccatggtcgaggtgagccccacgttctgcttcactctccccatctcccccccctccccacccccaattttgt
atttatttattttttaattattttgtgcagcgatgggggcggggggggggggggggcgcgcgccaggcggggggggggggcgaggggggggggggcga
ggcggagaggtgcggcggcagccaatcagagcggcgcgctccgaaagtttccttttatggcgaggcggcggcggcggcggccctataaaaagcgaagcgcg
cggcgggcgggagtcgctgcgacgctgccttcgccccgtgccccgctccgccgccgcctcgcgccgcccgccccggctctgactgaccgcgttactcccaca
ggtgagcggggggacggcccttctcctccgggctgtaattagctgagcaagaggtaagggtttaagggatggttggttggtggggtattaatgtttaattacctgg
agcacctgcctgaaatcactttttttcaggttggaccggtgccaccatggactataaggaccacgacggagactacaaggatcatgatattgattacaaagacgatg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cactcccactgtcctttcctaataaaatgaggaaattgcatcgcattgtctgagtaggtgtcattctattctggggggtggggggggcaggacagcaagggggagg
attgggaagagaatagcaggcatgctggggagcggccgcaggaacccctagtgatggagttggccactccctctctgcgcgctcgctcgctcactgaggccgg
gcgaccaaaggtcgcccgacgcccgggctttgcccgggcggcctcagtgagcgagcgagcgcgcagctgcctgcaggggcgcctgatgcggtattttctcctt
acgcatctgtgcggtatttcacaccgcatacgtcaaagcaaccatagtacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagctctaaatcgggggctcccttta
gggttccgatttagtgctttacggcacctcgaccccaaaaaacttgatttgggtgatggttcacgtagtgggccatcgccctgatagacggtttttcgccctttgacgtt
ggagtccacgttctttaatagtggactcttgttccaaactggaacaacactcaaccctatctcgggctattcttttgatttataagggattttgccgatttcggcctattggtt
aaaaaatgagctgatttaacaaaaatttaacgcgaattttaacaaaatattaacgtttacaattttatggtgcactctcagtacaatctgctctgatgccgcatagttaagc
cagccccgacacccgccaacacccgctgacgcgccctgacgggcttgtctgctcccggcatccgcttacagacaagctgtgaccgtctccgggagctgcatgtg
tcagaggttttcaccgtcatcaccgaaacgcgcgagacgaaagggcctcgtgatacgcctatttttataggttaatgtcatgataataatggtttcttagacgtcaggtg
gcacttttcggggaaatgtgcgcggaacccctatttgtttatttttctaaatacattcaaatatgtatccgctcatgagacaataaccctgataaatgcttcaataatattga
aaaaggaagagtatgagtattcaacatttccgtgtcgcccttattcccttttttgcggcattttgccttcctgtttttgctcacccagaaacgctggtgaaagtaaaagatg
ctgaagatcagttgggtgcacgagtgggttacatcgaactggatctcaacagcggtaagatccttgagagttttcgccccgaagaacgttttccaatgatgagcactt
ttaaagttctgctatgtggcgcggtattatcccgtattgacgccgggcaagagcaactcggtcgccgcatacactattctcagaatgacttggttgagtactcaccagt
cacagaaaagcatcttacggatggcatgacagtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggagctgaatgaagccataccaaacgacgagcgt
gacaccacgatgcctgtagcaatggcaacaacgttgcgcaaactattaactggcgaactacttactctagcttcccggcaacaattaatagactggatggaggcgg
ataaagttgcaggaccacttctgcgctcggcccttccggctggctggtttattgctgataaatctggagccggtgagcgtggaagccgcggtatcattgcagcactg
gggccagatggtaagccccccgtatcgtagttatctacacgacggggagtcaggcaactatggatgaacgaaatagacagatcgctgagataggtgcctcactg
attaagcattggtaactgtcagaccaagtttactcatatatactttagattgatttaaaacttcatttttaatttaaaaggatctaggtgaagatcctttttgataatctcatgac
caaaatcccttaacgtgagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgcagataccaaatactgtcct
tctagtgtagccgtagttaggccaccacttcaagaactctgtagcaccgcctacatacctcgctctgctaatcctgttaccagtggctgctgccagtggcgataagtc
gtgtcttaccgggttggactcaagacgatagttaccggataaggcgcagcggtcgggctgaacggggggttcgtgcacacagcccagcttggagcgaacgacct
acaccgaactgagatacctacagcgtgagctatgagaaagcgccacgcttcccgaagggagaaaggcggacaggtatccggtaagcggcagggtcggaaca
ggagagcgcacgagggagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgt
SEQ ID NO: 42
pBZ198-pU6- [symbol] - [symbol] -CBh-sv40NLS-Cas9-NLS-T2A-mCherry-P2A-S [symbol]
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaaCACCGCTGAAGCACTGCACGCCATgttttagagctagaaatagcaagttaaaataaggctagtccgttatcaacttga
aaaagtggcaccgagtcggtgcCGTACGGTGCGAGCGACCGAGAGAGGTCCCAAGCCATCAGCCTCAGCGCCT
CGAGCGCGAGAGCGGCGTTGCGCCGCTCTGGTTGAATTGCAGGACACTCTCCGCAAGGTAGCCTG

[symbol]

[symbol]

[symbol]

CCGTTTCCCCGGCCGGAGAGGTACTCACCGGAGGGGAGAGCCGGTGAGGCTACCGTGCCCCAGGT
GAGAAGGTGGTGCCTTCGGGCCTCCCTCGACCGCTCGCGCtttttttctagaggtacccgttacataacttacggtaaatggccc
gcctggctgaccgcccaacgacccccgcccattgacgtcaatagtaacgccaatagggactttccattgacgtcaatgggtggagtatttacggtaaactgcccact
tggcagtacatcaagtgtatcatatgccaagtacgccccctattgacgtcaatgacggtaaatggcccgcctggcattgtgcccagtacatgaccttatgggactttc
ctacttggcagtacatctacgtattagtcatcgctattaccatggtcgaggtgagccccacgttctgcttcactctccccatctcccccccctccccacccccaattttgt
atttatttattttttaattattttgtgcagcgatgggggcggggggggggggggggcgcgcgccaggcggggggggggggcgaggggggggggggcga
ggcggagaggtgcggcggcagccaatcagagcggcgcgctccgaaagtttccttttatggcgaggcggcggcggcggcggccctataaaaagcgaagcgcg
cggggggggagtcgctgcgacgctgccttcgccccgtgccccgctccgccgccgcctcgcgccgcccgccccggctctgactgaccgcgttactcccaca
ggtgagcggggggacggcccttctcctccgggctgtaattagctgagcaagaggtaagggtttaagggatggttggttggtggggtattaatgtttaattacctgg
agcacctgcctgaaatcactttttttcaggttggaccggtgccaccatggactataaggaccacgacggagactacaaggatcatgatattgattacaaagacgatg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cactcccactgtcctttcctaataaaatgaggaaattgcatcgcattgtctgagtaggtgtcattctattctggggggtggggggggcaggacagcaagggggagg
attgggaagagaatagcaggcatgctggggagcggccgcaggaacccctagtgatggagttggccactccctctctgcgcgctcgctcgctcactgaggccgg
gcgaccaaaggtcgcccgacgcccgggctttgcccgggcggcctcagtgagcgagcgagcgcgcagctgcctgcaggggcgcctgatgcggtattttctcctt
acgcatctgtgcggtatttcacaccgcatacgtcaaagcaaccataglacgcgccctgtagcggcgcattaagcgcggcgggtgtggtggtlacgcgcagcgtga
ccgctacacttgccagcgccctagcgcccgctcctttcgctttcttcccttcctttctcgccacgttcgccggctttccccgtcaagctctaaatcgggggctcccttta
gggttccgatttagtgctttacggcacctcgaccccaaaaaacttgatttgggtgatggttcacgtagtgggccatcgccctgatagacggtttttcgccctttgacgtt
ggagtccacgttctttaatagtggactcttgttccaaactggaacaacactcaaccctatctcgggctattcttttgatttataagggattttgccgatttcggcctattggtt
aaaaaatgagctgatttaacaaaaatttaacgcgaattttaacaaaatattaacgtttacaattttatggtgcactctcagtacaatctgctctgatgccgcatagttaagc
cagccccgacacccgccaacacccgctgacgcgccctgacgggcttgtctgctcccggcatccgcttacagacaagctgtgaccgtctccgggagctgcatgtg
tcagaggttttcaccgtcatcaccgaaacgcgcgagacgaaagggcctcgtgatacgcctatttttataggttaatgtcatgataataatggtttcttagacgtcaggtg
gcacttttcggggaaatgtgcgcggaacccctatttgtttatttttctaaatacattcaaatatgtatccgctcatgagacaataaccctgataaatgcttcaataatattga
aaaaggaagagtatgagtattcaacatttccgtgtcgcccttattcccttttttgcggcattttgccttcctgtttttgctcacccagaaacgctggtgaaagtaaaagatg
ctgaagatcagttgggtgcacgagtgggttacatcgaactggatctcaacagcggtaagatccttgagagttttcgccccgaagaacgttttccaatgatgagcactt
ttaaagttctgctatgtggcgcggtattatcccgtattgacgccgggcaagagcaactcggtcgccgcatacactattctcagaatgacttggttgagfactcaccagt
cacagaaaagcatcttacggatggcatgacagtaagagaattatgcagtgctgccataaccatgagtgataacactgcggccaacttacttctgacaacgatcgga
ggaccgaaggagctaaccgcttttttgcacaacatgggggatcatgtaactcgccttgatcgttgggaaccggagctgaatgaagccataccaaacgacgagcgt
gacaccacgatgcctgtagcaatggcaacaacgttgcgcaaactattaactggcgaactacttactctagcttcccggcaacaattaatagactggatggaggcgg
ataaagttgcaggaccacttctgcgctcggcccttccggctggctggtttattgctgataaatctggagccggtgagcgtggaagccgcggtatcattgcagcactg
gggccagatggtaagccctcccgtatcgtagttatctacacgacggggagtcaggcaactatggatgaacgaaatagacagatcgctgagataggtgcctcactg
attaagcattggtaactgtcagaccaagtttactcatatatactttagattgatttaaaacttcatttttaatttaaaaggatctaggtgaagatcctttttgataatctcatgac
caaaatcccttaacgtgagttttcgttccactgagcgtcagaccccgtagaaaagatcaaaggatcttcttgagatcctttttttctgcgcgtaatctgctgcttgcaaac
aaaaaaaccaccgctaccagcggtggtttgtttgccggatcaagagctaccaactctttttccgaaggtaactggcttcagcagagcgcagataccaaatactgtcct
tctagtgtagccgtagttaggccaccacttcaagaactctgtagcaccgcctacatacctcgctctgctaatcctgttaccagtggctgctgccagtggcgataagtc
gtgtcttaccgggttggactcaagacgatagttaccggataaggcgcagcggtcgggctgaacggggggttcgtgcacacagcccagcttggagcgaacgacct
acaccgaactgagatacctacagcgtgagctatgagaaagcgccacgcttcccgaagggagaaaggcggacaggtatccggtaagcggcagggtcggaaca
ggagagcgcacgagggagcttccagggggaaacgcctggtatctttatagtcctgtcgggtttcgccacctctgacttgagcgtcgatttttgtgatgctcgtcagg
ggggcggagcctatggaaaaacgccagcaacgcggcctttttacggttcctggccttttgctggccttttgctcacatgt
SEQ ID NO: 43
pBZ199-pU6-sgBFP- [symbol] -CBh-sv40NLS-Cas9- [symbol] NLS-T2A-mCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcCGTACGGTGCGAGCGACCGAGAGAGGTCCCAAGCCATCAGCCTCAGCGCCTC
GAGCGCGAGAGCGGCGTTGCGCCGCTCTGGTTGAATTGCAGGACACTCTCCGCAAGGTAGCCTGTT

[symbol]

[symbol]

[symbol]

TAGGCGCAACCGCCGTTTCCCCGGCCGGAGAGGTACTCACCGGAGGGGAGAGCCGGTGAGGCTAC
CGTGCCCCAGGTGAGAAGGTGGTGCCTTCGGGCCTCCCTCGACCGCTCGCGCtttttttctagaggtacccttaca
taacttacggtaaatggcccgcctggctgaccgcccaacgacccccgcccattgacgtcaatagtaacgccaatagggactttccattgacgtcaatgggggagt
atttacggtaaactgcccacttggcagtacatcaagtgtatcatatgccaagtacgccccctattgacgtcaatgacggtaaatggcccgcctggcattgtgcccagt
acatgaccttatgggactttcctacttggcagtacatctacgtattagtcatcgctattaccatggtcgaggtgagccccacgttctgcttcactctccccatctcccccc
cctccccacccccaattttgtatttatttattttttaattattttgtgcagcgatggggggggggggggggggggggggccaggggggggggggggg
aggggggggcggggcgaggcggagaggtgcggggcagccaatcagagcggcgcgctccgaaagtttccttttatggcgaggcggcggcggcggcggc
cctataaaaagcgaagcgcgcggcggggggagtcgctgcgacgctgccttcgccccgtgccccgctccgccgccgcctcgcgccgcccgccccggctctg
actgaccgcgttactcccacaggtgagcggggggacggcccttctcctccgggctgtaattagctgagcaagaggtaagggtttaagggatggttggttggtgg
ggtattaatgtttaattacctggagcacctgcctgaaatcactttttttcaggttggaccggtgccaccatggactataaggaccacgacggagactacaaggatcatg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ttgccagccatctgttgtttgcccctcccccgtgccttccttgaccctggaaggtgccactcccactgtcctttcctaataaaatgaggaaattgcatcgcattgcatcgcattgtctgag
taggtgtcattctattctggggggtggggggggcaggacagcaagggggaggattgggaagagaatagcaggcatgctggggagcggccgcaggaacccct
agtgatggagttggccactccctctctgcgcgctcgctcgctcactgaggccgggcgaccaaaggtcgcccgacgcccgggctttgcccgggcggcctcagtg
agcgagcgagcgcgcagctgcctgcaggggcgcctgatgcggtattttctccttacgcatctgtgcggtatttcacaccgcatacgtcaaagcaaccatagtacgc
gccctgtagcggcgcattaagcgcggcgggtgtggtggttacgcgcagcgtgaccgctacacttgccagcgccctagcgcccgctcctttcgctttcttcccttcct
ttctcgccacgttcgccggctttccccgtcaagctctaaatcgggggctccctttagggttccgatttagtgctttacggcacctcgaccccaaaaaacttgatttgggt
gatggttcacgtagtgggccatcgccctgatagacggtttttcgccctttgacgttggagtccacgttctttaatagtggactcttgttccaaactggaacaacactcaa
ccctatctcgggctattcttttgatttataagggattttgccgatttcggCctattggttaaaaaatgagctgatttaacaaaaatttaacgcgaattttaacaaaatattaac
gtttacaattttatggtgcactctcagtacaatctgctctgatgccgcatagttaagccagccccgacacccgccaacacccgctgacgcgccctgacgggcttgtct
gctcccggcatccgcttacagacaagctgtgaccgtctccgggagctgcatgtgtcagaggttttcaccgtcatcaccgaaacgcgcgagacgaaagggcctcgt
gatacgcctatttttataggttaatgtcatgataataatggtttcttagacgtcaggtggcacttttcggggaaatgtgcgcggaacccctatttgtttatttttctaaatacat
tcaaatatgtatccgctcatgagacaataaccctgataaatgcttcaataatattgaaaaaggaagagtatgagtattcaacatttccgtgtcgcccttattcccttttttgc
ggcattttgccttcctgtttttgctcacccagaaacgctggtgaaagtaaaagatgctgaagatcagttgggtgcacgagtgggttacatcgaactggatctcaacag
cggtaagatccttgagagttttcgccccgaagaacgttttccaatgatgagcacttttaaagttctgctatgtggcgcggtattatcccgtattgacgccgggcaagag
caactcggtcgccgcatacactattctcagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttgcacaacatgggggatcatgtaactc
gccttgatcgttgggaaccggagctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggcccttccggctggctggtttattg
ctgataaatctggagccggtgagcgtggaagccgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcatatatactttagattgattta
aaacttcatttttaatttaaaaggatctaggtgaagatcctttttgataatctcatgaccaaaatcccttaacgtgagttttcgttccactgagcgtcagaccccgtagaaa
agatcaaaggatcttcttgagatcctttttttctgcgcgtaatctgctgcttgcaaacaaaaaaaccaccgctaccagcggtggtttgtttgccggatcaagagctacca
actctttttccgaaggtaactggcttcagcagagcgcagataccaaatactgtccttctagtgtagccgtagttaggccaccacttcaagaactctgtagcaccgccta
catacctcgctctgctaatcctgttaccagtggctgctgccagtggcgataagtcgtgtcttaccgggttggactcaagacgatagttaccggataaggcgcagcgg
tcgggctgaacggggggttcgtgcacacagcccagcttggagcgaacgacctacaccgaactgagatacctacagcgtgagctatgagaaagcgccacgcttc
ccgaagggagaaaggcggacaggtatccggtaagcggcagggtcggaacaggagagcgcacgagggagcttccagggggaaacgcctggtatctttatagt
cctgtcgggtttcgccacctctgacttgagcgtcgatttttgtgatgctcgtcaggggggcggagcctatggaaaaacgccagcaacgcggcctttttacggttcctg
gccttttgctggccttttgctcacatgt
SEQ ID NO: 44
pBZ200-pU6-sgBFP- [symbol] CBh-sv40NLS-Cas9- [symbol] -NLS-T2A-mCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcCGTACGGTGCGAGCGACCGAGAGAGGTCCCAAGCCATCAGCCTCAGCGCCTC
GAGCGCGAGAGCGGCGTTGCGCCGCTCTGGTTGAATTGCAGGACACTCTCCGCAAGGTAGCCTGTT

[symbol]

[symbol]

[symbol]

CCTAGGCGCAACCGCCGTTTCCCCGGCCGGAGAGGTACTCACCGGAGGGGAGAGCCGGTGAGGCT
ACCGTGCCCCAGGTGAGAAGGTGGTGCCTTCGGGCCTCCCTCGACCGCTCGCGCtttttttctagaggtacccgtt
acataacttacggtaaatggcccgcctggctgaccgcccaacgacccccgcccattgacgtcaatagtaacgccaatagggactttccattgacgtcaatgggtgg
agtatttacggtaaactgcccacttggcagtacatcaagtgtatcatatgccaagtacgccccctattgacgtcaatgacggtaaatggcccgcctggcattgtgccc
agtacatgaccttatgggactttcctacttggcagtacatctacgtattagtcatcgctattaccatggtcgaggtgagccccacgttctgcttcactctccccatctccc
ccccctccccacccccaattttgtatttatttattttttaattattttgtgcagcgatggggggggggggggggggggggggccaggggggggggggg
gcgaggggggggcggggcgaggcggagaggtgcggcggcagccaatcagagcggcgcgctccgaaagtttccttttatggcgaggcggcggcggcggc
ggccctataaaaagcgaagcgcgcggcgggcgggagtcgctgcgacgctgccttcgccccgtgccccgctccgccgccgcctcgcgccgcccgccccggct
ctgactgaccgcgttactcccacaggtgagcggggggacggcccttctcctccgggctgtaattagctgagcaagaggtaagggtttaagggatggttggttggt
ggggtattaatgtttaattacctggagcacctgcctgaaatcactttttttcaggttggaccggtgccaccatggactataaggaccacgacggagactacaaggatc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ttgccagccatctgttgtttgcccctcccccgtgccttccttgaccctggaaggtgccactcccactgtcctttcctaataaaatgaggaaattgcatcgcattgtctgag
taggtgtcattctattctggggggtggggggggcaggacagcaagggggaggattgggaagagaatagcaggcatgctggggagcggccgcaggaacccct
agtgatggagttggccactccctctctgcgcgctcgctcgctcactgaggccgggcgaccaaaggtcgcccgacgcccgggctttgcccgggcggcctcagtg
agcgagcgagcgcgcagctgcctgcaggggcgcctgatgcggtattttctccttacgcatctgtgcggtatttcacaccgcatacgtcaaagcaaccatagtacgc
gccctgtagcggcgcattaagcgcggcgggtgtggtggtlacgcgcagcgtgaccgctacacttgccagcgccctagcgcccgctcctttcgctttcttcccttcct
ttctcgccacgttcgccggctttccccgtcaagctctaaatcgggggctccctttagggttccgatttagtgctttacggcacctcgaccccaaaaaacttgatttgggt
gatggttcacgtagtgggccatcgccctgatagacggtttttcgccctttgacgttggagtccacgttctttaatagtggactcttgttccaaactggaacaacactcaa
ccctatctcgggctattcttttgatttataagggattttgccgatttcggCctattggttaaaaaatgagctgatttaacaaaaatttaacgcgaattttaacaaaatattaac
gtttacaattttatggtgcactctcagtacaatctgctctgatgccgcatagttaagccagccccgacacccgccaacacccgctgacgcgccctgacgggcttgtct
gctcccggcatccgcttacagacaagctgtgaccgtctccgggagctgcatgtgtcagaggttttcaccgtcatcaccgaaacgcgcgagacgaaagggcctcgt
gatacgcctatttttataggttaatgtcatgataataatggtttcttagacgtcaggtggcacttttcggggaaatgtgcgcggaacccctatttgtttatttttctaaatacat
tcaaatatgtatccgctcatgagacaataaccctgataaatgcttcaataatattgaaaaaggaagagtatgagtattcaacatttccgtgtcgcccttattcccttttttgc
ggcattttgccttcctgtttttgctcacccagaaacgctggtgaaagtaaaagatgctgaagatcagttgggtgcacgagtgggttacatcgaactggatctcaacag
cggtaagatccttgagagttttcgccccgaagaacgttttccaatgatgagcacttttaaagttctgctatgtggcgcggtattatcccgtattgacgccgggcaagag
caactcggtcgccgcatacactattctcagaatgacttggttgagtactcaccagtcacagaaaagcatcttacggatggcatgacagtaagagaattatgcagtgct
gccataaccatgagtgataacactgcggccaacttacttctgacaacgatcggaggaccgaaggagctaaccgcttttttgcacaacatgggggatcatgtaactc
gccttgatcgttgggaaccggagctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggcccttccggctggctggtttattg
ctgataaatctggagccggtgagcgtggaagccgcggtatcattgcagcactggggccagatggtaagccctcccgtatcgtagttatctacacgacggggagtc
aggcaactatggatgaacgaaatagacagatcgctgagataggtgcctcactgattaagcattggtaactgtcagaccaagtttactcatatatactttagattgattta
aaacttcatttttaatttaaaaggatctaggtgaagatcctttttgataatctcatgaccaaaatcccttaacgtgagttttcgttccactgagcgtcagaccccgtagaaa
agatcaaaggatcttcttgagatccttttttttgcgcgtaatctgctgcttgcaaacaaaaaaaccaccgctaccagcggtggtttgtttgccggatcaagagctacca
actctttttccgaaggtaactggcttcagcagagcgcagataccaaatactgtccttctagtgtagccgtagttaggccaccacttcaagaactctgtagcaccgccta
catacctcgctctgctaatcctgttaccagtggctgctgccagtggcgataagtgtgtcttaccgggttggactcaagacgatagttaccggataaggcgcagcgg
tcgggctgaacggggggttcgtgcacacagcccagcttggagcgaacgacctacaccgaactgagatacctacagcgtgagctatgagaaagcgccacgcttc
ccgaagggagaaaggcggacaggtatccggtaagcggcagggtcggaacaggagagcgcacgagggagcttccagggggaaacgcctggtatctttatagt
cctgtcgggtttcgccacctctgacttgagcgtcgatttttgtgatgctcgtcaggggggcggagcctatggaaaaacgccagcaacgcggcctttttacggttcctg
gccttttgctggccttttgctcacatgt
SEQ ID NO: 45
pBZ201-pU6- [symbol] - [symbol] CBh-sv40NLS-Cas9- [symbol] -NLS-T2A-wCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcCGTACGGTGCGAGCGACCGAGAGAGGTCCCAAGCCATCAGCCTCAGCGCCTC
GAGCGCGAGAGCGGCGTTGCGCCGCTCTGGTTGAATTGCAGGACACTCTCCGCAAGGTAGCCTGTT

[symbol]

[symbol]

[symbol]

AACCGCCGTTTCCCCGGCCGGAGAGGTACTCACCGGAGGGGAGAGCCGGTGAGGCTACCGTGCCC
CAGGTGAGAAGGTGGTGCCTTCGGGCCTCCCTCGACCGCTCGCGCtttttttctagaggtacccgttacataacttacggta
aatggcccgcctggctgaccgcccaacgacccccgcccattgacgtcaatagtaacgccaatagggactttccattgacgtcaatgggtggagtatttacggtaaa
ctgcccacttggcagtacatcaagtgtatcatatgccaagtacgccccctattgacgtcaatgacggtaaatggcccgcctggcattgtgcccagtacatgaccttat
gggactttcctacttggcagtacatctacgtattagtcatcgctattaccatggtcgaggtgagccccacgttctgcttcactctccccatctcccccccctccccaccc
ccaattttgtatttatttattttttaattattttgtgcagcgatgggggcggggggggggggggggcgcgcgccaggcggggggggggggcgagggggggg
cggggcgaggcggagaggtgcggggcagccaatcagagcggcgcgctccgaaagtttccttttatggcgaggcggcggcggcggcggccctataaaaagc
gaagcgcgcggcgggcgggagtcgctgcgacgctgccttcgccccgtgccccgctccgccgccgcctcgcgccgcccgccccggctctgactgaccgcgtta
ctcccacaggtgagcggggggacggcccttctcctccgggctgtaattagctgagcaagaggtaagggtttaagggatggttggttggtggggtattaatgtttaa
ttacctggagcacctgcctgaaatcactttttttcaggttggaccggtgccaccatggactataaggaccacgacggagactacaaggatcatgatattgattacaaa

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gttgtttgcccctcccccgtgccttccttgaccctggaaggtgccactcccactgtcctttcctaataaaatgaggaaattgcatcgcattgtctgagtaggtgtcattct
attctggggggtggggggggcaggacagcaagggggaggattgggaagagaatagcaggcatgctggggagcggccgcaggaacccctagtgatggagtt
ggccactccctctctgcgcgctcgctcgctcactgaggccgggcgaccaaaggtcgcccgacgcccgggctttgcccgggcggcctcagtgagcgagcgagc
gcgcagctgcctgcaggggcgcctgatgcggtattttctccttacgcatctgtgcggtatttcacaccgcatacgtcaaagcaaccatagtacgcgccctgtagcgg
cgcattaagcgcggcgggtgtggtggttacgcgcagcgtgaccgcfacacttgccagcgccctagcgcccgctccttcgctttcttcccttcctttctcgccacgtt
cgccggctttccccgtcaagctctaaatcgggggctccctttagggttccgatttagtgctttacggcacctcgaccccaaaaaacttgatttgggtgatggttcacgt
agtgggccatcgccctgatagacggtttttcgccctttgacgttggagtccacgttctttaatagtggactcttgttccaaactggaacaacactcaaccctatctcggg
ctattcttttgatttataagggattttgccgatttcggCctattggttaaaaaatgagctgatttaacaaaaatttaacgcgaattttaacaaaatattaacgtttacaattttat
ggtgcactctcagtacaatctgctctgatgccgcatagttaagccagccccgacacccgccaacacccgctgacgcgccctgacgggcttgtctgctcccggcat
ccgcttacagacaagctgtgaccgtctccgggagctgcatgtgtcagaggttttcaccgtcatcaccgaaacgcgcgagacgaaagggcctcgtgatacgcctatt
tttataggttaatgtcatgataataatggtttcttagacgtcaggtggcacttttcggggaaatgtgcgcggaacccctatttgtttatttttctaaatacattcaaatatgtat
ccgctcatgagacaataaccctgataaatgcttcaataatattgaaaaaggaagagtatgagtattcaacatttccgtgtcgcccttattcccttttttgcggcattttgcct
tcctgtttttgctcacccagaaacgctggtgaaagtaaaagatgctgaagatcagttgggtgcacgagtgggttacatcgaactggatctcaacagcggtaagatcct
tgagagttttcgccccgaagaacgttttccaatgatgagcacttttaaagttctgctatgtggcgcggtattatcccgtattgacgccgggcaagagcaactcggtcgc
cgcatacactattctcagaatgacttggttgagtactcaccagtcacagaaaagcatcttacggatggcatgacagtaagagaattatgcagtgctgccataaccatg
agtgataacactgcggccaacttacttctgacaacgatcggaggaccgaaggagctaaccgcttttttgcacaacatgggggatcatgtaactcgccttgatcgttg
ggaaccggagctgaatgaagccataccaaacgacgagcgtgacaccacgatgcctgtagcaatggcaacaacgttgcgcaaactattaactggcgaactactta
ctctagcttcccggcaacaattaatagactggatggaggcggataaagttgcaggaccacttctgcgctcggcccttccggctggctggtttattgctgataaatctg
gagccggtgagcgtggaagccgcggtatcattgcagcactggggccagatggtaagccctcccgtatcgtagttatctacacgacggggagtcaggcaactatg
gatgaacgaaatagacagatcgctgagataggtgcctcactgattaagcattggtaactgtcagaccaagtttactcatatatactttagattgatttaaaacttcattttt
aatttaaaaggatctaggtgaagatcctttttgataatctcatgaccaaaatcccttaacgtgagttttcgttccactgagcgtcagaccccgtagaaaagatcaaagga
tcttcttgagatcctttttttctgcgcgtaatctgctgcttgcaaacaaaaaaaccaccgctaccagcggtggtttgtttgccggatcaagagctaccaactctttttccga
aggtaactggcttcagcagagcgcagataccaaatactgtccttctagtgtagccgtagttaggccaccacttcaagaactctgtagcaccgcctacatacctcgctc
tgctaatcctgttaccagtggctgctgccagtggcgataagtcgtgtcttaccgggttggactcaagacgatagttaccggataaggcgcagcggtcgggctgaac
ggggggttcgtgcacacagcccagcttggagcgaacgacctacaccgaactgagatacctacagcgtgagctatgagaaagcgccacgcttcccgaagggag
aaaggcggacaggtatccggtaagcggcagggtcggaacaggagagcgcacgagggagcttccagggggaaacgcctggtatctttatagtcctgtcgggttt
cgccacctctgacttgagcgtcgatttttgtgatgctcgtcaggggggcggagcctatggaaaaacgccagcaacgcggcctttttacggttcctggccttttgctgg
ccttttgctcacatgt
SEQ ID NO: 46
pBZ202-pU6- [symbol] - [symbol] CBh-sv40NLS-Cas9- [symbol] -NLS-T2A-mCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcCGTACGGTGCGAGCGACCGAGAGAGGTCCCAAGCCATCAGCCTCAGCGCCTC
GAGCGCGAGAGCGGCGTTGCGCCGCTCTGGTTGAATTGCAGGACACTCTCCGCAAGGTAGCCTGTT

[symbol]

[symbol]

[symbol]

GCAACCGCCGTTTCCCCGGCCGGAGAGGTACTCACCGGAGGGGAGAGCCGGTGAGGCTACCGTGC
CCCAGGTGAGAAGGTGGTGCCTTCGGGCCTCCCTCGACCGCTCGCGCtttttttctagaggtacccgttacataacttacg
gtaaatggcccgcctggctgaccgcccaacgacccccgcccattgacgtcaatagtaacgccaatagggactttccattgacgtcaatgggtggagtatttacggt
aaactgcccacttggcagtacatcaagtgtatcatatgccaagtacgccccctattgacgtcaatgacggtaaatggcccgcctggcattgtgcccagtacatgacct
tatgggactttcctacttggcagtacatctacgtattagtcatcgctattaccatggtcgaggtgagccccacgttctgcttcactctccccatctcccccccctccccac
ccccaattttgtatttatttattttttaattattttgtgcagcgatgggggcggggggggggggggggcgcgcgccaggcggggggggggggcgagggggg
ggcggggcgaggcggagaggtgcggcggcagccaatcagagcggcgcgctccgaaagtttccttttatggcgaggcggcggcggcggcggccctataaaaa
gcgaagcgcgcggcgggcgggagtcgctgcgacgctgccttcgccccgtgccccgctccgccgccgcctcgcgccgcccgccccggctctgactgaccgcg
ttactcccacaggtgagcggggggacggcccttctcctccgggctgtaattagctgagcaagaggtaagggtttaagggatggttggttggtggggtattaatgtt
taattacctggagcacctgcctgaaatcactttttttcaggttggaccggtgccaccatggactataaggaccacgacggagactacaaggatcatgatattgattaca

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

tctgttgtttgcccctcccccgtgccttccttgaccctggaaggtgccactcccactgtcctttcctaataaaatgaggaaattgcatcgcattgtctgagtaggtgtcatt
ctattctggggggtggggtggggcaggacagcaagggggaggattgggaagagaatagcaggcatgctggggagcggccgcaggaacccctagtgatgga
gttggccactccctctctgcgcgctcgctcgctcactgaggccgggcgaccaaaggtcgcccgacgcccgggctttgcccgggcggcctcagtgagcgagcga
gcgcgcagctgcctgcaggggcgcctgatgcggtattttctccttacgcatctgtgcggtatttcacaccgcatacgtcaaagcaaccatagtacgcgccctgtagc
ggcgcattaagcgcggcgggtgtggtggttacgcgcagcgtgaccgctacacttgccagcgccctagcgcccgctcctttcgctttcttcccttcctttctcgccac
gttcgccggctttccccgtcaagctctaaatcgggggctccctttagggttccgatttagtgctttacggcacctcgaccccaaaaaacttgatttgggtgatggttcac
gtagtgggccatcgccctgatagacggtttttcgccctttgacgttggagtccacgttctttaatagtggactcttgttccaaactggaacaacactcaaccctatctcg
ggctattcttttgatttataagggattttgccgatttcggCctattggttaaaaaatgagctgatttaacaaaaatttaacgcgaattttaacaaaatattaacgtttacaattt
tatggtgcactctcagtacaatctgctctgatgccgcatagttaagccagccccgacacccgccaacacccgctgacgcgccctgacgggcttgtctgctcccggc
atccgcttacagacaagctgtgaccgtctccgggagctgcatgtgtcagaggttttcaccgtcatcaccgaaacgcgcgagacgaaagggcctcgtgatacgcct
atttttataggttaatgtcatgataataatggtttcttagacgtcaggtggcacttttcggggaaatgtgcgcggaacccctatttgtttatttttctaaatacattcaaatatg
tatccgctcatgagacaataaccctgataaatgcttcaataatattgaaaaaggaagagtatgagtattcaacatttccgtgtcgcccttattcccttttttgcggcattttg
ccttcctgtttttgctcacccagaaacgctggtgaaagtaaaagatgctgaagatcagttgggtgcacgagtgggttacatcgaactggatctcaacagcggtaagat
ccttgagagttttcgccccgaagaacgttttccaatgatgagcacttttaaagttctgctatgtggcgcggtattatcccgtattgacgccgggcaagagcaactcggt
cgcgcatacactattctcagaatgacttggttgagtactcaccagtcacagaaaagcatcttacggatggcatgacagtaagagaattatgcagtgctgccataacc
atgagtgataacactgcggccaacttacttctgacaacgatcggaggaccgaaggagctaaccgcttttttgcacaacatgggggatcatgtaactcgccttgatcg
ttgggaaccggagctgaatgaagccataccaaacgacgagcgtgacaccacgatgcctgtagcaatggcaacaacgttgcgcaaactattaactggcgaactact
tactctagcttcccggcaacaattaatagactggatggaggcggataaagttgcaggaccacttctgcgctcggcccttccggctggctggtttattgctgataaatct
ggagccggtgagcgtggaagccgcggtatcattgcagcactggggccagatggtaagccctcccgtatcgtagttatctacacgacggggagtcaggcaactat
ggatgaacgaaatagacagatcgctgagataggtgcctcactgattaagcattggtaactgtcagaccaagtttactcatatatactttagattgatttaaaacttcatttt
taatttaaaaggatctaggtgaagatcctttttgataatctcatgaccaaaatcccttaacgtgagttttcgttccactgagcgtcagaccccgtagaaaagatcaaagg
atcttcttgagatcctttttttctgcgcgtaatctgctgcttgcaaacaaaaaaaccaccgctaccagcggtggtttgtttgccggatcaagagctaccaactctttttccg
aaggtaactggcttcagcagagcgcagataccaaatactgtccttctagtgtagccgtagttaggccaccacttcaagaactctgtagcaccgcctacatacctcgct
ctgctaatcctgttaccagtggctgctgccagtggcgataagtcgtgtcttaccgggttggactcaagacgatagttaccggataaggcgcagcggtcgggctgaa
cggggggttcgtgcacacagcccagcttggagcgaacgacctacaccgaactgagatacctacagcgtgagctatgagaaagcgccacgcttcccgaaggga
gaaaggcggacaggtatccggtaagcggcagggtcggaacaggagagcgcacgagggagcttccagggggaaacgcctggtatctttatagtcctgtcgggtt
tcgccacctctgacttgagcgtcgatttttgtgatgctcgtcaggggggggagcctatggaaaaacgccagcaacgcggcctttttacggttcctggccttttgctg
gccttttgctcacatgt
SEQ ID NO: 47
pBZ203-pU6- [symbol] - [symbol] _CBh-sv40NLS-Cas9- [symbol] -NLS-T2A-mCherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcCGTACGGTGCGAGCGACCGAGAGAGGTCCCAAGCCATCAGCCTCAGCGCCTC
GAGCGCGAGAGCGGCGTTGCGCCGCTCTGGTTGAATTGCAGGACACTCTCCGCAAGGTAGCCTGTT

[symbol]

[symbol]

[symbol]

CGTTTCCCCGGCCGGAGAGGTACTCACCGGAGGGGAGAGCCGGTGAGGCTACCGTGCCCCAGGTG
AGAAGGTGGTGCCTTCGGGCCTCCCTCGACCGCTCGCGCtttttttctagaggtacccgttacataacttacggtaaatggcccg
cctggctgaccgcccaacgacccccgcccattgacgtcaatagtaacgccaatagggactttccattgacgtcaatgggtggagtatttacggtaaactgcccactt
ggcagtacatcaagtgtatcatatgccaagtacgccccctattgacgtcaatgacggtaaatggcccgcctggcattgtgcccagtacatgaccttatgggactttcc
tacttggcagtacatctacgtattagtcatcgctattaccatggtcgaggtgagccccacgttctgcttcactctccccatctcccccccctccccacccccaattttgta
tttatttattttttaattattttgtgcagcgatggggggggggggggggggggggggccaggggggggggggcagggggggggggg
ggcggagaggtgcggcggcagccaatcagagcggcgcgctccgaaagtttccttttatggcgaggcggcggcggcggcggccctataaaaagcgaagcgcg
cggcgggcgggagtcgctgcgacgctgccttcgccccgtgccccgctccgccgccgcctcgcgccgcccgccccggctctgactgaccgcgttactcccaca
ggtgagcgggcgggacggcccttctcctccgggctgtaattagctgagcaagaggtaagggtttaagggatggttggttggtggggtattaatgtttaattacctgg
agcacctgcctgaaatcactttttttcaggttggaccggtgccaccatggactataaggaccacgacggagactacaaggatcatgatattgattacaaagacgatg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ccctcccccgtgccttccttgaccctggaaggtgccactcccactgtcctttccctaataaaatgaggaaattgcatcgcattgtcgagtaggtgtcattctattctggg
gggtggggtggggcaggacagcaagggggaggattgggaagagaatagcaggcatgctggggagcggccgcaggaacccctagtgatggagttggccact
ccctctctgcgcgctcgctcgctcactgaggccgggcgaccaaaggtcgcccgacgcccgggctttgcccgggcggcctcagtgagcgagcgagcgcgcag
ctgcctgcaggggcgcctgatgcggtattttctccttacgcatctgtgcggtatttcaccgcatacgtcaaagcaaccatagtacgcgccctgtagcggcgcatta
agcgcggcgggtgtggtggttacgcgcagcgtgaccgctacacttgccagcgccctagcgcccgctcctttcgctttcttcccttcctttctcgccacgttcgccgg
ctttccccgtcaagctctaaatcgggggctccctttagggttccgatttagtgctttacggcacctcgaccccaaaaaacttgatttgggtgatggttcacgtagtgggc
catcgccctgatagacggtttttcgccctttgacgttggagtccacgttcctttaatagtggactcttgttccaaactggaacaacactcaaccctatctcgggctattctttt
gatttataagggattttgccgatttcggCctattggttaaaaaatgagctgatttaacaaaaatttaacgcgaattttaacaaaatattaacgtttacaattttatggtgcact
ctcagtacaatctgctctgatgccgcatagttaagccagccccgacacccgccaacacccgctgacgcgccctgacgggcttgtctgctcccggcatccgcttaca
gacaagctgtgaccgtctccgggagctgcatgtgtcagaggttttcaccgtcatcaccgaaacgcgcgagacgaaagggcctcgtgatacgcctatttttataggtt
aatgtcatgataataatggtttcttagacgtcaggtggcacttttcggggaaatgtgcgcggaacccctatttgtttatttttctaaatacattcaaatatgtatccgctcatg
agacaataaccctgataaatgcttcaataatattgaaaaaggaagagtatgagtattcaacatttccgtgtcgcccttattcccttttttgcggcattttgccttcctgtttt
gctcacccagaaacgctggtgaaagtaaaagatgctgaagatcagttgggtgcacgagtgggttacatcgaactggatctcaacagcggtaagatccttgagagtt
ttcgccccgaagaacgttttccaatgatgagcacttttaaagttctgctatgtggcgcggtattatcccglattgacgccgggcaagagcaactcggtcgccgcatac
actattctcagaatgacttggttgagtactcaccagtcacagaaaagcatcttacggatggcatgacagtaagagaattatgcagtgctgccataaccatgagtgata
acactgcggccaactlacttctgacaacgatcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaactattaactggcgaactacttactctagctt
cccggcaacaattaatagactggatggaggcggataaagttgcaggaccacttctgcgctcggcccttccggctggctggtttattgctgataaatctggagccggt
gagcgtggaagccgcggtatcattgcagcactggggccagatggtaagccctcccgtatcgtagttatctacacgacggggagtcaggcaactatggatgaacg
aaatagacagatcgctgagataggtgcctcactgattaagcattggtaactgtcagaccaagtttactcatatatactttagattgatttaaaacttcatttttaatttaaaa
ggatctaggtgaagatcctttttgataatctcatgaccaaaatcccttaacgtgagttttcgttccactgagcgtcagaccccgtagaaaagatcaaaggatcttcttga
gatcctttttttctgcgcgtaatctgctgcttgcaaacaaaaaaaccaccgctaccagcggtggtttgtttgccggatcaagagctaccaactctttttccgaaggtaact
ggcttcagcagagcgcagataccaaatactgtccttctagtgtagccgtagttaggccaccacttcaagaactctgtagcaccgcctacatacctcgctctgctaatc
ctgttaccagtggctgctgccagtggcgataagtcgtgtcttaccgggttggactcaagacgatagttaccggataaggcgcagcggtcgggctgaacggggggt
tcgtgcacacagcccagcttggagcgaacgacctacaccgaactgagatacctacagcgtgagctatgagaaagcgccacgcttcccgaagggagaaaggcg
gacaggtatccggtaagcggcagggtcggaacaggagagcgcacgagggagcttccagggggaaacgcctggtatctttatagtcctgtcgggtttcgccacct
ctgacttgagcgtcgatttttgtgatgctcgtcaggggggcggagcctatggaaaaacgccagcaacgcggcctttttacggttcctggccttttgctggccttttgct
cacatgt
SEQ ID NO: 48
pBZ204-pU6-sgBFP-Dn CBh-sv40NLS-Cas9-89/6387-NLS-T2A-Cherry
gagggcctatttcccatgattccttcatatttgcatatacgatacaaggctgttagagagataattggaattaatttgactgtaaacacaaagatattagtacaaaatacgt
gacgtagaaagtaataatttcttgggtagtttgcagttttaaaattatgttttaaaatggactatcatatgcttaccgtaacttgaaagtatttcgatttcttggctttatatatct
tgtggaaaggacgaaacaccGCTGAAGCACTGCACGCCATgttttagagctagaaatagcaagttaaaataaggctagtccgttatcaacttgaaa
aagtggcaccgagtcggtgcCGTACGGTGCGAGCGACCGAGAGAGGTCCCAAGCCATCAGCCTCAGCGCCTC
GAGCGCGAGAGCGGCGTTGCGCCGCTCTGGTTGAATTGCAGGACACTCTCCGCAAGGTAGCCTGTT

[symbol]

[symbol]

[symbol]

CGTTTCCCCGGCCGGAGAGGTACTCACCGGAGGGGAGAGCCGGTGAGGCTACCGTGCCCCAGGTG
AGAAGGTGGTGCCTTCGGGCCTCCCTCGACCGCTCGCGCtttttttctagaggtacccgttacataacttacggtaaatggcccg
cctggctgaccgcccaacgacccccgcccattgacgtcaatagtaacgccaatagggactttccattgacgtcaatgggtggagtatttacggtaaactgcccactt
ggcagtacatcaagtgtatcatatgccaagtacgccccctattgacgtcaatgacggtaaatggcccgcctggcattgtgcccagtacatgaccttatgggactttcc
tacttggcagtacatctacgtattagtcatcgctattaccatggtcgaggtgagccccacgttctgcttcactctccccattcccccccctccccacccccaattttgta
tttatttattttttaattattttgtgcagcgatgggggcggggggggggggggggcgcgcgccaggcggggggggggggcgaggggggggggggcga
ggcggagaggtgcggggcagccaatcagagcggcgcgctccgaaagtttccttttatggcgaggcggcggcggcggcggccctataaaaagcgaagcgcg
cggcggggggagtcgctgcgacgctgccttcgccccgtgccccgctccgccgccgcctcgcgccgcccgccccggctctgactgaccgcgttactcccaca
ggtgagcggggggacggcccttctcctccgggctgtaattagctgagcaagaggtaagggtttaagggatggttggttggtggggtattaatgtttaattacctgg
agcacctgcctgaaatcactttttttcaggttggaccggtgccaccatggactataaggaccacgacggagactacaaggatcatgatattgattacaaagacgatg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ccctcccccgtgccttccttgaccctggaaggtgccactcccactgtcctttcctaataaaatgaggaaattgcatcgcattgtctgagtaggtgtcattctattctggg
gggtggggtggggcaggacagcaagggggaggattgggaagagaatagcaggcatgctggggagcggccgcaggaacccctagtgatggagttggccact
ccctctctgcgcgctcgctcgctcactgaggccgggcgaccaaaggtcgcccgacgcccgggctttgcccgggcggcctcagtgagcgagcgagcgcgcag
ctgcctgcaggggcgcctgatgcggtattttcctcctacgcatctgtgcggtatttcacaccgcatacgtcaaagcaaccatagtacgcgccctgtagcggcgcatta
agcgcggcgggtgtggtggttacgcgcagcgtgaccgctacacttgccagcgccctagcgcccgctcctttcgctttcttcccttcctttctcgccacgttcgccgg
ctttccccgtcaagctctaaatcgggggctccctttagggttccgatttagtgctttacggcacctcgaccccaaaaaacttgatttgggtgatggttcacgtagtgggc
catcgccctgatagacggtttttcgccctttgacgttggagtccacgttctttaatagtggactcttgttccaaactggaacaacactcaaccctatctcgggctattctttt
gatttataagggattttgccgatttcggCctattggttaaaaaatgagctgatttaacaaaaatttaacgcgaattttaacaaatattaacgtttacaattttatggtgcact
ctcagtacaatctgctctgatgccgcatagttaagccagccccgacacccgccaacacccgctgacgcgccctgacgggcttgtctgctcccggcatccgcttaca
gacaagctgtgaccgtctccgggagctgcatgtgtcagaggttttcaccgtcatcaccgaaacgcgcgagacgaaagggcctcgtgatacgcctatttttataggtt
aatgtcatgataataatggtttcttagacgtcaggtggcacttttcggggaaatgtgcgcggaacccctatttgtttatttttctaaatacattcaaatatgtatccgctcatg
agacaataaccctgataaatgcttcaataatattgaaaaaggaagagtatgagtattcaacatttccgtgtcgcccttattcccttttttgcggcattttgccttcctgttttt
gctcacccagaaacgctggtgaaagtaaaagatgctgaagatcagttgggtgcacgagtgggttacatcgaactggatctcaacagcggtaagatccttgagagtt
ttcgccccgaagaacgttttccaatgatgagcacttttaaagttctgctatgtggcgcggtattatcccglattgacgccgggcaagagcaactcggtcgccgcatac
actattctcagaatgacttggttgagtactcaccagtcacagaaaagcatcttacggatggcatgacagtaagagaattatgcagtgctgccataaccatgagtgata
acactgcggccaacttacttctgacaacgatcggaggaccgaaggagctaaccgcttttttgcacaacatgggggatcatgtaactcgccttgatcgttgggaaccg
gagctgaatgaagccataccaaacgacgagcgtgacaccacgatgcctgtagcaatggcaacaacgttgcgcaaactattaactggcgaactacttactctagctt
cccggcaacaattaatagactggatggaggggataaagttgcaggaccacttctgcgctcggcccttccggctggctggtttattgctgataaatctggagccggt
gagcgtggaagccgcggtatcattgcagcactggggccagatggtaagccctcccgtatcgtagttatctacacgacggggagtcaggcaactatggatgaacg
aaatagacagatcgctgagataggtgcctcactgattaagcattggtaactgtcagaccaagtttactcatatatactttagattgatttaaaacttcatttttaatttaaaa
ggatctaggtgaagatcctttttgataatctcatgaccaaaatcccttaacgtgagttttcgttccactgagcgtcagaccccgtagaaaagatcaaaggatcttcttga
gatcctttttttctgcgcgtaatctgctgcttgcaaaaaaaaaaccaccgctaccagcggtggtttgtttgccggatcaagagctaccaactctttttccgaaggtaact
ggcttcagcagagcgcagataccaaatactgtccttctagtgtagccgtagttaggccaccacttcaagaactctgtagcaccgcctacatacctcgctctgctaatc
ctgttaccagtggctgctgccagtggcgataagtcgtgtcttaccgggttggactcaagacgatagttaccggataaggcgcagcggtcgggctgaacggggggt
tcgtgcacacagcccagcttggagcgaacgacctacaccgaactgagatacctacagcgtgagctatgagaaagcgccacgcttcccgaagggagaaaggcg
gacaggtatccggtaagcggcagggtcggaacaggagagcgcacgagggagcttccagggggaaacgcctggtatctttatagtcctgtcgggtttcgccacct
ctgacttgagcgtcgatttttgtgatgctcgtcaggggggcggagcctatggaaaaacgccagcaacgcggcctttttacggttcctggccttttgctggccttttgct
cacatgt
SEQ ID NO: 49
pBZ207-pU6- [symbol] - [symbol] _CBh-sv40NLS-Cas9- [symbol] -NLS-TZA-mCherry
catgtgagggcctatttcccatgattccttcatatttgcatatacgatacaaggctgttagagagataattggaattaatttgactgtaaacacaaagatattagtacaaaa
tacgtgacgtagaaagtaataatttcttgggtagtttgcagttttaaaattatgttttaaaatggactatcatatgcttaccgtaacttgaaagtatttcgatttcttggctttat
atatcttgtggaaaggacgaaacaccGCTGAAGCACTGCACGCCATgttttagagctagaaatagcaagttaaaataaggctagtccgttatcaactt
gaaaaagtggcaccgaglcggtgcCGTACGATGCGCACCCTTAGCGAGAGGTTTATCATTAAGGTCAACCTCTG

[symbol]

[symbol]

[symbol]

AACCCGTTTCTTCTGACGTAAGGGTGCGCAtttttttctagaggtacccgttacataacttacggtaaatggcccgcctggctgaccgcc
caacgacccccgcccattgacgtcaatagtaacgccaatagggactttccattgacgtcaatgggggagtatttacggtaaactgcccacttggcagtacatcaag
tgtatcatatgccaagtacgccccctattgacgtcaatgacggtaaatggcccgcctggcattgtgcccagtacatgaccttatgggactttcctacttggcagtacat
ctacgtattagtcatcgctattaccatggtcgaggtgagccccacgttctgcttcactctccccatctcccccccctccccacccccaattttgtatttatttattttttaatta
ttttgtgcagcgatgggggcggggggggggggggggcgcgcgccaggggggcggggggggcgaggggggggggggcgaggcggagaggtgcg
gcggcagccaatcagagcggcgcgctccgaaagtttccttttatggcgaggcggcggcggcggcggccctataaaaagcgaagcgcgcggggggggagt
cgctgcgacgctgccttcgccccgtgccccgctccgccgccgcctcgcgccgcccgccccggctctgactgaccgcgttactcccacaggtgagcgggggg
acggcccttctcctccgggctgtaattagctgagcaagaggtaagggtttaagggatggttggttggtggggtattaatgtttaattacctggagcacctgcctgaaat
cactttttttcaggttggaccggtgccaccatggactataaggaccacgacggagactacaaggatcatgatattgattacaaagacgatgacgataagatggcccc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ttcctagagctcgctgatcagcctcgactgtgccttctagttgccagccatctgttgtttgcccctcccccgtgccttccttgaccctggaaggtgccactcccactgtc
ctttcctaataaaatgaggaaattgcatcgcattgtctgagtaggtgtcattctattctggggggtggggggggcaggacagcaagggggaggattgggaagaga
atagcaggcatgctggggagcggccgcaggaacccctagtgatggagttggccactccctcttgcgcgctcgctcgctcactgaggccggggaccaaaggt
cgcccgacgcccgggctttgcccgggcggcctcagtgagcgagcgagcgcgcagctgcctgcaggggcgcctgatgcggtattttctccttacgcatctgtgcg
gtatttcacaccgcatacgtcaaagcaaccatagtacgcgccctgtagcggcgcattaagcgcggcgggtgtggtggttacgcgcagcgtgaccgctacacttgc
cagcgccctagcgcccgctcctttcgctttcttcccttcctttctcgccacgttcgccggctttccccgtcaagctctaaatcgggggctccctttagggttccgatttag
tgctttacggcacctcgaccccaaaaaacttgatttgggtgatggttcacgtagtgggccatcgccctgatagacggtttttcgccctttgacgttggagtccacgttct
ttaatagtggactcttgttccaaactggaacaacactcaaccctatctcgggctattcttttgatttataagggattttgccgatttcggCctattggttaaaaaatgagct
gatttaacaaaaatttaacgcgaattttaacaaaatattaacgtttacaattttatggtgcactctcagtacaatctgctctgatgccgcatagttaagccagccccgaca
cccgccaacacccgctgacgcgccctgacgggcttgtctgctcccggcatccgcttacagacaagctgtgaccgtctccgggagctgcatgtgtcagaggttttca
ccgtcatcaccgaaacgcgcgagacgaaagggcctcgtgatacgcctatttttataggttaatgtcatgataataatggtttcttagacgtcaggtggcacttttcggg
gaaatgtgcgcggaacccctatttgtttatttttctaaatacattcaaatatgtatccgctcatgagacaataaccctgataaatgcttcaataatattgaaaaaggaagag
tatgagtattcaacatttccgtgtcgcccttattcccttttttgcggcattttgccttcctgtttttgctcacccagaaacgctggtgaaagtaaaagatgctgaagatcagtt
gggtgcacgagtgggttacatcgaactggatctcaacagcggtaagatccttgagagttttcgccccgaagaacgttttccaatgatgagcacttttaaagttctgcta
tctggcgcggtattatcccgtattgacgccgggcaagagcaactcggtcgccgcatacactattctcagaatgacttggttgagtactcaccagtcacagaaaagca
tcttacggatggcatgacagtaagagaattatgcagtgctgccataaccatgagtgataacactgcggccaacttacttctgacaacgatcggaggaccgaaggag
ctaaccgcttttttgcacaacatgggggatcatgtaactcgccttgatcgttgggaaccggagctgaatgaagccataccaaacgacgagcgtgacaccacgatgc
ctgtagcaatggcaacaacgttgcgcaaactattaactggcgaactacttactctagcttcccggcaacaattaatagactggatggaggggataaagttgcagga
ccacttctgcgctcggcccttccggctggctggtttattgctgataaatctggagccggtgagcgtggaagccgcggtatcattgcagcactggggccagatggta
agccctcccgtatcgtagttatctacacgacggggagtcaggcaactatggatgaacgaaatagacagatcgctgagataggtgcctcactgattaagcattggtaa
ctgtcagaccaagtttactcatatatactttagattgatttaaaacttcatttttaatttaaaaggatctaggtgaagatcctttttgataatctcatgaccaaaatcccttaac
gtgagttttcgttccactgagcgtcagaccccgtagaaaagatcaaaggatcttcttgagatcctttttttctgcgcgtaatctgctgcttgcaaacaaaaaaaccaccg
ctaccagcggtggtttgtttgccggatcaagagctaccaactctttttccgaaggtaactggcttcagcagagcgcagataccaaatactgttcttctagtgtagccgt
agttaggccaccacttcaagaactctgtagcaccgcctacatacctcgctctgctaatcctgttaccagtggctgctgccagtggcgataagtcgtgtcttaccgggtt
ggactcaagacgatagttaccggataaggcgcagcggtcgggctgaacggggggttcgtgcacacagcccagcttggagcgaacgacctacaccgaactgag
atacctacagcgtgagctatgagaaagcgccacgcttcccgaagggagaaaggcggacaggtatccggtaagcggcagggtcggaacaggagagcgcacga
gggagcttccagggggaaacgcctggtatctttatagtcctgtcgggtttcgccacctctgacttgagcgtcgatttttgtgatgctcgtcaggggggcggagcctat
ggaaaaacgccagcaacgcggc
SEO ID NO: 50
pBZ208-pU6- [symbol] - [symbol] _CBh-sv40NLS-Cas9- [symbol] -NLS-T2A-sxCherry
catgtgagggcctatttcccatgattccttcatatttgcatatacgatacaaggctgttagagagataattggaattaatttgactgtaaacacaaagatattagtacaaaa
tacgtgacgtagaaagtaataatttcttgggtagtttgcagttttaaaattatgttttaaaatggactatcatatgcttaccgtaacttgaaagtatttcgatttcttggctttat
atatcttgtggaaaggacgaaacaccGCTGAAGCACTGCACGCCATgttttagagctagaaatagcaagttaaaataaggctagtccgttatcaactt
gaaaaagtggcaccgagtcggtgcCGTACGATGCGCACCCTTAGCGAGAGGTTTATCATTAAGGTCAACCTCTG

[symbol]

[symbol]

[symbol]

GAACCCGTTTCTTCTGACGTAAGGGTGCGCAtttttttctagaggtacccgttacataacttacggtaaatggcccgcctggctgaccgc
ccaacgacccccgcccattgacgtcaatagtaacgccaatagggactttccattgacgtcaatgggggagtatttacggtaaactgcccacttggcagtacatcaa
gtgtatcatatgccaagtacgccccctattgacgtcaatgacggtaaatggcccgcctggcattgtgcccagtacatgaccttatgggactttcctacttggcagtaca
tctacgtattagtcatcgctattaccatggtcgaggtgagccccacgttctgcttcactctccccatctcccccccctccccacccccaattttgtatttatttattttttaatt
attttgtgcagcgatgggggcggggggggggggggggcgcgcgccaggcggggcggggcggggcgaggggcggggcggggcgaggcggagaggtgc
ggcggcagccaatcagagcggcgcgctccgaaagtttccttttatggcgaggggggggcggcggccctataaaaagcgaagcgcgcggcgggcggga
gtcgctgcgacgctgccttcgccccgtgccccgctccgccgccgcctcgcgccgcccgccccggctctgactgaccgcgttactcccacaggtgagcgggcgg
gacggcccttctcctccgggctgtaattagctgagcaagaggtaagggtttaagggatggttggttggtggggtattaatgtttaattacctggagcacctgcctgaa
atcactttttttcaggttggaccggtgccaccatggactataaggaccacgacggagactacaaggatcatgatattgattacaaagacgatgacgataagatggcc

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

ttcctagagctcgctgatcagcctcgactgtgccttctagttgccagccatctgttgtttgcccctcccccgtgccttccttgaccctggaaggtgccactcccactgtc
ctttcctaataaaatgaggaaattgcatcgcattgtctgagtaggtgtcattctattctggggggtggggtggggcaggacagcaagggggaggattgggaagaga
atagcaggcatgctggggagcggccgcaggaacccctagtgatggagttggccactccctctctgcgcgctcgctcgctcactgaggccgggcgaccaaaggt
cgcccgacgcccgggctttgcccgggcggcctcagtgagcgagcgagcgcgcagctgcctgcaggggcgcctgatgcggtattttctccttacgcatctgtgcg
gtatttcacaccgcatacgtcaaagcaaccatagtacgcgccctgtagcggcgcattaagcgcggcgggtgtggtggttacgcgcagcgtgaccgctacacttgc
cagcgccctagcgcccgctcctttcgctttcttcccttcctttctcgccacgttcgccggctttccccgtcaagctctaaatcgggggctccctttagggttccgatttag
tgctttacggcacctcgaccccaaaaaacttgatttgggtgatggttcacgtagtgggccatcgccctgatacgacggtttttcgccctttgacgttggagtccacgttct
ttaatagtggactcttgttccaaactggaacaacactcaaccctatctcgggctattcttttgatttataagggattttgccgatttcggCctattggttaaaaaatgagct
gatttaacaaaaatttaacgcgaattttaacaaaatattaacgtttacaattttatggtgcactcagtacaatctgctctgatgccgcatagttaagccagccccgaca
cccgccaacacccgctgacgcgccctgacgggcttgtctgctcccggcatccgcttacagacaagctgtgaccgtctccgggagctgcatgtgtcagaggttttca
ccgtcatcaccgaaacgcgcgagacgaaagggcctcgtgatacgcctatttttataggttaatgtcatgataataatggtttcttagacgtcaggtggcacttttcggg
gaaatgtgcgcggaacccctatttgtttatttttctaaatacattcaaatatgtatccgctcatgagacaataaccctgataaatgcttcaataatattgaaaaaggaagag
tatgagtattcaacatttccgtgtcgcccttattcccttttttgcggcattttgccttcctgtttttgctcacccagaaacgctggtgaaagtaaaagatgctgaagatcagtt
gggtgcacgagtgggttacatcgaactggatctcaacagcggtaagatccttgagagttttcgccccgaagaacgttttccaatgatgagcacttttaaagttctgcta
tgtggcgcggtattatcccgtattgacgccgggcaagagcaactcggtcgccgcatacactattctcagaatgacttggttgagtactcaccagtcacagaaaagca
tcttacggatggcatgacagtaagagaattatgcagtgctgccataaccatgagtgataacactgcggccaacttacttctgacaacgatcggaggaccgaaggag
ctaaccgcttttttgcacaacatgggggatcatgtaactcgccttgatcgttgggaaccggagctgaatgaagccataccaaacgacgagcgtgacaccacgatgc
ctgtagcaatggcaacaacgttgcgcaaactattaactggcgaactacttactctagcttcccggcaacaattaatagactggatggaggcggataaagttgcagga
ccacttctgcgctcggcccttccggctggctggtttattgctgataaatctggagccggtgagcgtggaagccgcggtatcattgcagcactggggccagatggta
agccctcccgtatcgtagttatctacacgacggggagtcaggcaactatggatgaacgaaatagacagatcgctgagataggtgcctcactgattaagcattggtaa
ctgtcagaccaagtttactcatatatactttagattgatttaaaacttcatttttaatttaaaaggatctaggtgaagatcctttttgataatctcatgaccaaaatcccttaac
gtgagttttcgttccactgagcgtcagaccccgtagaaaagatcaaaggatcttcttgagatccttttttctgcgcgtaatctgctgcttgcaaacaaaaaaaccaccg
ctaccagcggtggtttgtttgccggatcaagagctaccaactctttttccgaaggtaactggcttcagcagagcgcagcataccaaatactgttcttctagtgtagccgt
agttaggccaccacttcaagaactctgtagcaccgcctacatacctcgctctgctaatcctgttaccagtggctgctgccagtggcgataagtcgtgtccttaccgggtt
ggactcaagacgatagttaccggataaggcgcagcggtcgggctgaacggggggttcgtgcacacagcccagcttggagcgaaccgacctacaccgaactgag
atacctacagcgtgagctatgagaaagcgccacgcttcccgaagggagaaaggcggacaggtatccggtaagcggcagggtcggaacaggagagcgcacga
gggagcttccagggggaaacgcctggtatctttatagtcctgtcgggtttcgccacctctgacttgagcgtcgatttttgtgatgctcgtcaggggggcggagcctat
ggaaaaacgccagcaacgcggc
SEQ ID NO: 51
pBZ209-pU6- [symbol] - [symbol] _CBh-sv40NLS-Cas9- [symbol] -NLS-T2A-mCherry
catgtgagggcctatttcccatgattccttcatatttgcatatacgatacaaggctgttagagagataattggaattaatttgactgtaaacacaaagatattagtacaaaa
tacgtgacgtagaaagtaataatttcttgggtagtttgcagttttaaaattatgttttaaaatggactatcatatgcttaccgtaacttgaaagtatttcgatttcttggctttat
atatcttgtggaaaggacgaaacaccGCTGAAGCACTGCACGCCATgttttagagctagaaatagcaagttaaaataaggctagtccgttatcaactt
gaaaaagtggcaccgagtcggtgcCGTACGATGCGCACCCTTAGCGAGAGGTTTATCATTAAGGTCAACCTCTG

[symbol]

[symbol]

[symbol]

TCTTCTGACGTAAGGGTGCGCAtttttttctagaggtacccgttacataacttacggtaaatggcccgcctggctgaccgcccaacgacccccg
cccattgacgtcaatagtaacgccaatagggactttccattgacgtcaatgggtggagtatttacggtaaactgcccacttggcagtacatcaagtgtatcatatgcca
agtacgccccctattgacgtcaatgacggtaaatggcccgcctggcattgtgcccagtacatgaccttatgggactttcctacttggcagtacatctacgtattagtcat
cgctattaccatggtcgaggtgagccccacgttctgcttcactctccccatctcccccccctccccacccccaattttgtatttatttattttttaattattttgtgcagcgat
gggggcggggggggggggggggcgcgcgccaggcggggcggggcggggcgaggggcggggcggggcgaggcggagaggtgcggcggcagccaagt
cagagcggcgcgctccgaaagtttccttttatggcgaggggcggcggcggcggccctataaaaagcgaagcgcgcggggggggagtcgctgcgacgct
gccttcgccccgtgccccgctccgcgccgcctcgcgccgcccgccccggctctgactgaccgcgttactcccacaggtgagcggggggacggcccttctcc
tccgggctgtaattagctgagcaagaggtaagggtttaagggatggttggttggtggggtattaatgtttaattacctggagcacctgcctgaaatcactttttttcaggt
tggaccggtgccaccatggactataaggaccacgacggagactacaaggatcatgatattgattacaaagacgatgacgataagatggccccaaagaagaagcg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

cagcctcgactgtgccttctagttgccagccatctgttgtttgcccctcccccgtgccttccttgaccctggaaggtgccactcccactgtcctttcctaataaaatgag
gaaattgcatcgcattgtctgagtaggtgtcattctattctggggggtggggtggggcaggacagcaagggggaggattgggaagagaatagcaggcatgctgg
ggagcggccgcaggaacccctagtgatggagttggccactccctctctgcgcgctcgctcgctcactgaggccgggcgaccaaaggtcgcccgacgcccggg
ctttgcccgggcggcctcagtgagcgagcgagcgcgcagctgcctgcaggggcgcctgatgcggtattttctccttacgcatctgtgcggtatttcacaccgcata
cgtcaaagcaaccatagtacgcgccctgtagcggcgcatlaagcgcggcgggtgtggtggttacgcgcagcgtgaccgctacacttgccagcgccctagcgcc
cgctcctttcgctttcttcccttcctttctcgccacgttcgccggctttccccgtcaagctctaaatcgggggctccctttagggttccgatttagtgctttacggcacctc
gaccccaaaaaacttgatttgggtgatggttcacgtagtgggccatcgccctgatagacggtttttcgccctttgacgttggagtccacgttctttaatagtggactcttg
ttccaaactggaacaacactcaaccctatctcgggctattcttttgatttataagggattttgccgatttcggCctattggttaaaaaatgagctgatttaacaaaaatttaa
cgcgaattttaacaaaatattaacgtttacaattttatggtgcactctcagtacaatctgctctgatgccgcatagttaagccagccccgacacccgccaacacccgct
gacgcgccctgacgggcttgtctgctcccggcatccgcttacagacaagctgtgaccgtctccgggagctgcatgtgtcagaggttttcaccgtcatcaccgaaac
gcgcgagacgaaagggcctcgtgatacgcctatttttataggttaatgtcatgataataatggtttcttagacgtcaggtggcacttttcggggaaatgtgcgcggaac
ccctatttgtttatttttctaaatacattcaaatatgtatccgctcatgagacaataaccctgataaatgcttcaataatattgaaaaaggaagagtatgagtattcaacattt
ccgtgtcgcccttattcccttttttgcggcattttgccttcctgtttttgctcacccagaaacgctggtgaaagtaaaagatgctgaagatcagttgggtgcacgagtggg
ttacatcgaactggatctcaacagcggtaagatccttgagagttttcgccccgaagaacgttttccaatgatgagcacttttaaagttctgctatgtggcgcggtattatc
ccgtattgacgccgggcaagagcaactcggtcgccgcatacactattctcagaatgacttggttgagtactcaccagtcacagaaaagcatcttacggatggcatg
acagtaagagaattatgcagtgctgccataaccatgagtgataacactgcggccaacttacttctgacaacgatcggaggaccgaaggagctaaccgcttttttgca
caacatgggggatcatgtaactcgccttgatcgttgggaaccggagctgaatgaagccataccaaacgacgagcgtgacaccacgatgcctgtagcaatggcaa
caacgttgcgcaaactattaactggcgaactacttactctagcttcccggcaacaattaatagactggatggaggcggataaagttgcaggaccacttctgcgctcg
gcccttccggctggctggtttattgctgataaatctggagccggtgagcgtggaagccgcggtatcattgcagcactggggccagatggtaagccctcccgtatcg
tagttatctacacgacggggagtcaggcaactatggatgaacgaaatagacagatcgctgagataggtgcctcactgattaagcattggtaactgtcagaccaagtt
tactcatatatactttagattgatttaaaacttcatttttaatttaaaaggatctaggtgaagatcctttttgataatctcatgaccaaaatcccttaacgtgagttttcgttccac
tgagcgtcagaccccgtagaaaagatcaaaggatcttcttgagatcctttttttctgcgcgtaatctgctgcttgcaaacaaaaaaaccaccgctaccagcggtggttt
gtttgccggatcaagagctaccaactctttttccgaaggtaactggcttcagcagagcgcagataccaaatactgttcttctagtgtagccgtagttaggccaccactt
caagaactctgtagcaccgcctacatacctcgctctgctaatcctgttaccagtggctgctgccagtggcgataagtcgtgtcttaccgggttggactcaagacgata
gttaccggataaggcgcagcggtcgggctgaacggggggttcgtgcacacagcccagcttggagcgaacgacctacaccgaactgagatacctacagcgtga
gctatgagaaagcgccacgcttcccgaagggagaaaggcggacaggtatccggtaagcggcagggtcggaacaggagagcgcacgagggagcttccaggg
ggaaacgcctggtatctttatagtcctgtcgggtttcgccacctctgacttgagcgtcgatttttgtgatgctcgtcagggggggcggagcctatggaaaaacgccagc
aacgcggc
SEQ ID NO: 52
pBZ210-pU6- [symbol] - [symbol] _CBh-sv40NLS-Cas9- [symbol] -NLS-T2A-mCherry
catgtgagggcctatttcccatgattccttcatatttgcatatacgatacaaggctgttagagagataattggaattaatttgactgtaaacacaaagatattagtacaaaa
tacgtgacgtagaaagtaataatttcttgggtagtttgcagttttaaaattatgttttaaaatggactatcatatgcttaccgtaacttgaaagtatttcgatttcttggctttat
atatcttgtggaaaggacgaaacaccGCTGAAGCACTGCACGCCATgttttagagctagaaatagcaagttaaaataaggctagtcgttatcaactt
gaaaaagtggcaccgagtcggtgcCGTACGATGCGCACCCTTAGCGAGAGGTTTATCATTAAGGTCAACCTCTG

[symbol]

[symbol]

[symbol]

TTTCTTCTGACGTAAGGGTGCGCAtttttttctagaggtacccgttacataacttacggtaaatggcccgcctggctgaccgcccaacgacccc
cgcccattgacgtcaatagtaacgccaatagggactttccattgacgtcaatgggtggagtatttacggtaaactgcccacttggcagtacatcaagtgtatcatatgc
caagtacgccccctattgacgtcaatgacggtaaatggcccgcctggcattgtgcccagtacatgaccttatgggactttcctacttggcagtacatctacgtattagt
catcgctattaccatggtcgaggtgagccccacgttctgcttcactctccccatctcccccccctccccacccccaattttgtatttatttattttttaattattttgtgcagcg
atggggcggggggggggggggggcgcgcgccaggcggggcggggcggggcgaggggcggggcggggcgaggcggagaggtgcggcggcagcca
atcagagcggcgcgctccgaaagtttccttttatggcgaggcggcggcggcggcggccctataaaaagcgaagcgcgcggcgggcgggagtcgctgcgacg
ctgccttcgccccgtgccccgctccgccgccgcctcgcgccgcccgccccggctctgactgaccgcgttactcccacaggtgagcgggcgggacggcccttct
cctccgggctgtaattagctgagcaagaggtaagggtttaagggatggttggttggtggggtattaatgtttaattacctggagcacctgcctgaaatcactttttttca
ggttggaccggtgccaccatggactataaggaccacgacggagactacaaggatcatgatattgattacaaagacgatgacgataagatggccccaaagaagaa

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

tgatcagcctcgactgtgccttctagttgccagccatctgttgtttgcccctcccccgtgccttccttgaccctggaaggtgccactcccactgtcctttcctaataaaat
gaggaaattgcatcgcattgtctgagtaggtgtcattctattctggggggtggggggggcaggacagcaagggggaggattgggaagagaatagcaggcatgc
tggggagcggccgcaggaacccctagtgatggagttggccactccctctctgcgcgctcgctcgctcactgaggccgggcgaccaaaggtcgcccgacgccc
gggctttgcccgggcggcctcagtgagcgagcgagcgcgcagctgcctgcaggggcgcctgatgcggtattttctccttacgcatctgtgcggtatttcacaccgc
atacgtcaaagcaaccatagtacgcgccctgtagcggcgcattaagcgcgggggtgtggtggttacgcgcagcgtgaccgctacacttgccagcgccctagcg
cccgctcctttcgctttcttcccttcctttctcgccacgttcgccggctttccccgtcaagctctaaatcgggggctccctttagggttccgatttagtgctttacggcacct
cgaccccaaaaaacttgatttgggtgatggttcacgtagtgggccatcgccctgatagacggtttttcgccctttgacgttggagtccacgttctttaatagtggactctt
gttccaaactggaacaacactcaaccctatctcgggctattcttttgatttataagggattttgccgatttcggCctattggttaaaaaatgagctgatttaacaaaaattt
aacgcgaattttaacaaaatattaacgtttacaattttatggtgcactctcagtacaatctgctctgatgccgcatagttaagccagccccgacacccgccaacacccg
ctgacgcgccctgacgggcttgtctgctcccggcatccgcttacagacaagctgtgaccgtctccgggagctgcatgtgtcagaggttttcaccgtcatcaccgaa
acgcgcgagacgaaagggcctcgtgatacgcctatttttataggttaatgtcatgataataatggtttcttagacgtcaggtggcacttttcggggaaatgtgcgcgga
acccctatttgtttatttttctaaatacattcaaatatgtatccgctcatgagacaataaccctgataaatgcttcaataatattgaaaaaggaagagtatgagtattcaacat
ttccgtgtcgcccttattcccttttttgcggcattttgccttcctgtttttgctcacccagaaacgctggtgaaagtaaaagatgctgaagatcagttgggtgcacgagtg
ggttacatcgaactggatctcaacagcggtaagatccttgagagttttcgccccgaagaacgttttccaatgatgagcacttttaaagttctgctatgtggcgcggtatt
atcccgtattgacgccgggcaagagcaactcggtcgccgcatacactattctcagaatgacttggttgagtactcaccagtcacagaaaagcatcttacggatggca
tgacagtaagagaattatgcagtgctgccataaccatgagtgataacactgcggccaacttacttctgacaacgatcggaggaccgaaggagctaaccgcttttttg
cacaacatgggggatcatgtaactcgccttgatcgttgggaaccggagctgaatgaagccataccaaacgacgagcgtgacaccacgatgcctgtagcaatggc
aacaacgttgcgcaaactattaactggcgaactacttactctagcttcccggcaacaattaatagactggatggaggcggataaagttgcaggaccacttctgcgct
cggcccttccggctggctggtttattgctgataaatctggagccggtgagcgtggaagccgcggtatcattgcagcactggggccagatggtaagccctcccgtat
cgtagttatctacacgacggggagtcaggcaactatggatgaacgaaatagacagatcgctgagataggtgcctcactgattaagcattggtaactgtcagaccaa
gtttactcatatatactttagattgatttaaaacttcatttttaatttaaaaggatctaggtgaagatcctttttgataatctcatgaccaaaatcccttaacgtgagttttcgttc
cactgagcgtcagaccccgtagaaaagatcaaaggatcttcttgagatcctttttttctgcgcgtaatctgctgcttgcaaacaaaaaaaccaccgctaccagcggtg
gtttgtttgccggatcaagagctaccaactctttttccgaaggtaactggcttcagcagagcgcagataccaaatactgttcttctagtgtagccgtagttaggccacca
cttcaagaactctgtagcaccgcctacatacctcgctctgctaatcctgttaccagtggctgctgccagtggcgataagtcgtgtcttaccgggttggactcaagacg
atagttaccggataaggcgcagcggtcgggctgaacggggggttcgtgcacacagcccagttggagcgaacgacctacaccgaactgagatacctacagcgt
gagctatgagaaagcgccacgcttcccgaagggagaaaggcggacaggtatccggtaagcggcagggtcggaacaggagagcgcacgagggagcttccag
ggggaaacgcctggtatctttatagtcctgtcgggtttcgccacctctgacttgagcgtcgatttttgtgatgctcgtcaggggggcggagcctatggaaaaacgcca
gcaacgcggc
SEO ID NO: 53
pBZ211-pU6- [symbol] - [symbol] _CBh-sv40NLS-Cas9- [symbol] -NLS-TZA-mCherry
catgtgagggcctatttcccatgattccttcatatttgcatatacgatacaaggctgttagagagataattggaattaatttgactgtaaacacaaagatattagtacaaaa
tacgtgacgtagaaagtaataatttcttgggtagtttgcagttttaaaattatgttttaaaatggactatcatatgcttaccgtaacttgaaagtatttcgatttcttggctttat
atatcttgtggaaaggacgaaacaccGCTGAAGCACTGCACGCCATgttttagagctagaaatagcaagttaaaataaggctagtccgttatcaactt
gaaaaagtggcaccgagtcggtgcCGTACGATGCGCACCCTTAGCGAGAGGTTTATCATTAAGGTCAACCTCTG

[symbol]

[symbol]

[symbol]

ACGTAAGGGTGCGCAtttttttctagaggtacccgttacataacttacggtaaatggcccgcctggctgaccgcccaacgacccccgcccattgacgt
caatagtaacgccaatagggactttccattgacgtcaatgggggagtatttacggtaaactgcccacttggcagtacatcaagtgtatcatalgccaagtacgcccc
ctattgacgtcaatgacggtaaatggcccgcctggcattgtgcccagtacatgaccttatgggactttcctacttggcagtacatctacgtattagtcatcgctattacca
tggtcgaggtgagccccacgttctgcttcactctccccatctcccccccctccccacccccaattttgtatttatttattttttaattattttgtgcagcgatgggggggg
gggggggggggggcgcgcgccaggcggggcggggcggggcgaggggcggggcggggcgaggcggagaggtgcggcggcagccaatcagagcggc
gcgctccgaaagtttccttttatggcgaggcggcggcggcggcggccctataaaaagcgaagcgcgcggcgggcgggagtcgctgcgacgctgccttcgccc
cgtgccccgctccgccgccgcctcgcgccgcccgccccggctctgactgaccgcgttactcccacaggtgagcgggcgggacggcccttctcctccgggctgt
aattagctgagcaagaggtaagggtttaagggatggttggttggtggggtattaatgtttaattacctggagcacctgcctgaaatcactttttttcaggttggaccggt
gccaccatggactataaggaccacgacggagactacaaggatcatgatattgattacaaagacgatgacgataagatggccccaaagaagaagcggaaggtcg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gccttctagttgccagccatctgttgtttgcccctcccccgtgccttccttgaccctggaaggtgccactcccactgtcctttcctaataaaatgaggaaattgcatcgc
attgtctgagtaggtgtcattctattctggggggtggggggggcaggacagcaagggggaggattgggaagagaatagcaggcatgctggggagcggccgca
ggaacccctagtgatggagttggccactccctctctgcgcgctcgctcgctcactgaggccgggcgaccaaaggtcgcccgacgcccgggctttgcccgggcg
gcctcagtgagcgagcgagcgcgcagctgcctgcaggggcgcctgatgcggtattttctccttacgcatctgtgcggtatttcacaccgcatacgtcaaagcaacc
atagtacgcgccctgtagcggcgcattaagcgcggcgggtgtggtggtlacgcgcagcgtgaccgctacacttgccagcgccctagcgcccgctcctttcgctttc
ttcccttcctttctcgccacgttcgccggctttccccgtcaagctctaaatcgggggctccctttagggttccgatttagtgctttacggcacctcgaccccaaaaaactt
gatttgggtgatggttcacgtagtgggccatcgccctgatagacggtttttcgccctttgacgttggagtccacgttctttaatagtggactcttgttccaaactggaaca
acactcaaccctatctcgggctattcttttgatttataagggattttgccgatttcggCctattggttaaaaaatgagctgatttaacaaaaatttaacgcgaattttaacaa
aatattaacgtttacaattttatggtgcactctcagtacaatctgctctgatgccgcatagttaagccagccccgacacccgccaacacccgctgacgcgccctgacg
ggcttgtctgctcccggcatccgcttacagacaagctgtgaccgtctccgggagctgcatgtgtcagaggttttcaccgtcatcaccgaaacgcgcgagacgaaag
ggcctcgtgatacgcctatttttataggttaatgtcatgataataatggtttcttagacgtcaggtggcacttttcggggaaatgtgcgcggaacccctatttgtttatttttc
taaatacattcaaatatgtatccgctcatgagacaataaccctgataaatgcttcaataatattgaaaaaggaagagtatgagtattcaacatttccgtgtgcccttattc
ccttttttgcggcattttgccttcctgtttttgctcacccagaaacgctggtgaaagtaaaagatgctgaagatcagttgggtgcacgagtgggttacatcgaactggat
ctcaacagcggtaagatccttgagagttttcgccccgaagaacgttttccaatgatgagcacttttaaagttctgctatgtggcgcggtattatcccgtattgacgccgg
gcaagagcaactcggtcgccgcatacactattctcagaatgacttggttgagtactcaccagtcacagaaaagcatcttacggatggcatgacagtaagagaattat
gcagtgctgccataaccatgagtgataacactgcggccaacttacttctgacaacgatcggaggaccgaaggagctaaccgcttttttgcacaacatgggggatca
tgtaactcgccttgatcgttgggaaccggagctgaatgaagccataccaaacgacgagcgtgacaccacgatgcctgtagcaatggcaacaacgttgcgcaaact
attaactggcgaactacttactctagcttcccggcaacaattaatagactggatggaggggataaagttgcaggaccacttctgcgctcggcccttccggctggct
ggtttattgctgataaatctggagccggtgagcgtggaagccgcggtatcattgcagcactggggccagatggtaagccccccgtatcgtagttatctacacgacg
gggagtcaggcaactatggatgaacgaaatagacagatcgctgagataggtgcctcactgattaagcattggtaactgtcagaccaagtttactcatatatactttag
attgatttaaaacttcatttttaatttaaaaggatctaggtgaagatcctttttgataatctcatgaccaaaatcccttaacgtgagttttcgttccactgagcgtcagacccc
gtagaaaagatcaaaggatcttcttgagatcctttttttctgcgcgtaatctgctgcttgcaaacaaaaaaaccaccgctaccagcggtggtttgtttgccggatcaaga
gctaccaactctttttccgaaggtaactggcttcagcagagcgcagataccaaatactgttcttctagtgtagccgtagttaggccaccacttcaagaactctgtagca
ccgcclacatacctcgctctgctaatcctgttaccagtggctgctgccagtggcgataagtcgtgtcttaccgggttggactcaagacgatagttaccggataaggcg
cagcggtcgggctgaacggggggttcgtgcacacagcccagcttggagcgaacgacctacaccgaactgagatacctacagcgtgagctatgagaaagcgcc
acgcttcccgaagggagaaaggcggacaggtatccggtaagcggcagggtcggaacaggagagcgcacgagggagcttccagggggaaacgcctggtatct
ttatagtcctgtcgggtttcgccacctctgacttgagcgtcgatttttgtgatgctcgtcaggggggcggagcctatggaaaaacgccagcaacgcggc
SEQ ID NO: 54
pBZ212-pU6- [symbol] - [symbol] _CBh-sv40NLS-Cas9- [symbol] -NLS-T2A-mCherry
catgtgagggcctatttcccatgattccttcatatttgcatatacgatacaaggctgttagagagataattggaattaatttgactgtaaacacaaagatattagtacaaaa
tacgtgacgtagaaagtaataatttcttgggtagtttgcagttttaaaattatgttttaaaatggactatcatatgcttaccgtaacttgaaagtatttcgatttcttggctttat
atatcttgtggaaaggacgaaacaccGCTGAAGCACTGCACGCCATgttttagagctagaaatagcaagttaaaataaggctagtccgttatcaactt
gaaaaagtggcaccgagtcggtgcCGTACGATGCGCACCCTTAGCGAGAGGTTTATCATTAAGGTCAACCTCTG

[symbol]

[symbol]

[symbol]

GACGTAAGGGTGCGCAtttttttctagaggtacccgttacataacttacggtaaatggcccgcctggctgaccgcccaacgacccccgcccattgac
gtcaatagtaacgccaatagggactttccattgacgtcaatgggtggagtatttacggtaaactgcccacttggcagtacatcaagtgtatcatatgccaagtacgcc
ccctattgacgtcaatgacggtaaatggcccgcctggcattgtgcccagtacatgaccttatgggactttcctacttggcagtacatctacgtattagtcatcgctattac
catggtcgaggtgagccccacgttctgcttcactctccccatctcccccccctccccacccccaattttgtatttatttattttttaattattttgtgcagcgatgggggcgg
ggggggggggggggcgcgcgccaggcggggcggggcggggcgaggggcggggcggggcgaggcggagaggtgcggcggcagccaatcagagcgg
cgcgctccgaaagtttccttttatggcgaggcggcggcggcggcggccctataaaaagcgaagcgcgcggcgggcgggagtcgctgcgacgctgccttcgcc
ccgtgccccgctccgccgccgcctcgcgccgcccgccccggctctgactgaccgcgttactcccacaggtgagcgggcgggacggcccttctcctccgggctg
taattagctgagcaagaggtaagggtttaagggatggttggttggtggggtattaatgtttaattacctggagcacctgcctgaaatcactttttttcaggttggaccggt
gccaccatggactataaggaccacgacggagactacaaggatcatgatattgattacaaagacgatgacgataagatggccccaaagaagaagcggaaggtcg

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

[symbol]

gccttctagttgccagccatctgttgtttgcccctcccccgtgccttccttgaccctggaaggtgccactcccactgtcctttcctaataaaatgaggaaattgcatcgc
attgtctgagtaggtgtcattctattctggggggtggggggggcaggacagcaagggggaggattgggaagagaatagcaggcatgctggggagcggccgca
ggaacccctagtgatggagttggccactccctctctgcgcgctcgctcgctcactgaggccgggcgaccaaaggtcgcccgacgcccgggctttgcccgggcg
gcctcagtgagcgagcgagcgcgcagctgcctgcaggggcgcctgatgcggtattttctccttacgcatctgtgcggtatttcacaccgcatacgtcaaagcaacc
atagtacgcgccctgtagcggcgcattaagcgcggcgggtgtggtggttacgcgcagcgtgaccgctacacttgccagcgccctagcgcccgctcctttcgctttc
ttcccttcctttctcgccacgttcgccggctttccccgtcaagctctaaatcgggggctccctttagggttccgatttagtgctttacggcacctcgaccccaaaaaactt
gatttgggtgatggttcacgtagtgggccatcgccctgatagacggtttttcgccctttgacgttggagtccacgttctttaatagtggactcttgttccaaactggaaca
acactcaaccctatctcgggctattcttttgatttataagggattttgccgatttcggCctattggttaaaaaatgagctgatttaacaaaaatttaacgcgaattttaacaa
aatattaacgtttacaattttatggtgcactctcagtacaatctgctctgatgccgcatagttaagccagccccgacacccgccaacacccgctgacgcgccctgacg
ggcttgtctgctcccggcatccgcttacagacaagctgtgaccgtctccgggagctgcatgtgtcagaggttttcaccgtcatcaccgaaacgcgcgagacgaaag
ggcctcgtgatacgcctatttttataggttaatgtcatgataataatggtttcttagacgtcaggtggcacttttcggggaaatgtgcgcggaacccctatttgtttatttttc
taaatacattcaaatatgtatccgctcatgagacaataaccctgataaatgcttcaataatattgaaaaaggaagagtatgagtattcaacatttccgtgtcgcccttattc
ccttttttgcggcattttgccttcctgtttttgctcacccagaaacgctggtgaaagtaaaagatgctgaagatcagttgggtgcacgagtgggttacatcgaactggat
ctcaacagcggtaagatccttgagagttttcgccccgaagaacgttttccaatgatgagcacttttaaagttctgctatgtggcgcggtattatcccgtattgacgccgg
gcaagagcaactcggtcgccgcatacactattctcagaatgacttggttgagtactcaccagtcacagaaaagcatcttacggatggcatgacagtaagagaattat
gcagtgctgccataaccatgagtgataacactgcggccaacttacttctgacaacgatcggaggaccgaaggagctaaccgttttttgcacaacatgggggatca
tgtaactcgccttgatcgttgggaaccggagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggaccacttctgcgctcggcccttccggctggct
ggtttattgctgataaatctggagccggtgagcgtggaagccgcggtatcattgcagcactggggccagatggtaagccctcccgtatcgtagttatctacacgacg
gggagtcaggcaactatggatgaacgaaatagacagatcgctgagataggtgcctcactgattaagcattggtaactgtcagaccaagtttactcatatatactttag
attgatttaaaacttcatttttaatttaaaaggatctaggtgaagatcctttttgataatctcatgaccaaaatcccttaacgtgagttttcgttccactgagcgtcagacccc
gtagaaaagatcaaaggatcttcttgagatcctttttttctgcgcgtaatctgctgcttgcaaacaaaaaaaccaccgctaccagcggtggtttgtttgccggatcaaga
gctaccaactctttttccgaaggtaactggcttcagcagagcgcagataccaaatactgttcttctagtgtagccgtagttaggccaccacttcaagaactctgtagca
ccgcctacatacctcgctctgctaatcctgttaccagtggctgctgccagtggcgataagtcgtgtcttaccgggttggactcaagacgatagttaccggataaggcg
cagcggtcgggctgaacggggggttcgtgcacacagcccagcttggagcgaacgacctacaccgaactgagatacctacagcgtgagctatgagaaagcgcc
acgcttcccgaagggagaaaggcggacaggtatccggtaagcggcagggtcggaacaggagagcgcacgagggagcttccagggggaaacgcctggtatct
ttatagtcctgtcgggtttcgccacctctgacttgagcgtcgatttttgtgatgctcgtcaggggggcggagcctatggaaaaacgccagcaacgcggc
SEQ ID NO: 55
RNA polymerase III promoter U6
GAGGGCCTATTTCCCATGATTCCTTCATATTTGCATATACGATACAAGGCTGTTAGAGAGATAATT
GGAATTAATTTGACTGTAAACACAAAGATATTAGTACAAAATACGTGACGTAGAAAGTAATAATT
TCTTGGGTAGTTTGCAGTTTTAAAATTATGTTTTAAAATGGACTATCATATGCTTACCGTAACTTGA
AAGTATTTCGATTTCTTGGCTTTATATATCTTGTGGAAAGGACGAAACACC
SEQ ID NO: 56
RNA polymerase II promoter CBh
CGTTACATAACTTACGGTAAATGGCCCGCCTGGCTGACCGCCCAACGACCCCCGCCCATTGACGTC
AATAGTAACGCCAATAGGGACTTTCCATTGACGTCAATGGGTGGAGTATTTACGGTAAACTGCCCA
CTTGGCAGTACATCAAGTGTATCATATGCCAAGTACGCCCCCTATTGACGTCAATGACGGTAAATG
GCCCGCCTGGCATTGTGCCCAGTACATGACCTTATGGGACTTTCCTACTTGGCAGTACATCTACGT
ATTAGTCATCGCTATTACCATGGTCGAGGTGAGCCCCACGTTCTGCTTCACTCTCCCCATCTCCCCC
CCCTCCCCACCCCCAATTTTGTATTTATTTATTTTTTAATTATTTTGTGCAGCGATGGGGGCGGGGG
GGGGGGGGGGGGCGCGCCAGGCGGGGGGGGCGGGGCGAGGGGGGGGGGGGGCGAGGCGGA
GAGGTGCGGCGGCAGCCAATCAGAGCGGCGCGCTCCGAAAGTTTCCTTTTATGGCGAGGCGGCGG
CGGCGGCGGCCCTATAAAAAGCGAAGCGCGCGGCGGGCGGGAGTCGCTGCGACGCTGCCTTCGCC
CCGTGCCCCGCTCCGCCGCCGCCTCGCGCCGCCCGCCCCGGCTCTGACTGACCGCGTTACTCCCAC
AGGTGAGCGGGCGGGACGGCCCTTCTCCTCCGGGCTGTAATTAGCTGAGCAAGAGGTAAGGGTTT
AAGGGATGGTTGGTTGGTGGGGTATTAATGTTTAATTACCTGGAGCACCTGCCTGAAATCACTTTT
TTTCAG
SEQ ID NO: 57
Nucleoplasmin nuclear localization sequence (NLS)
AAAAGGCCGGCGGCCACGAAAAAGGCCGGCCAGGCAAAAAAGAAAAAG
SEO ID NO: 58
Simian virus 40 (SV40) nuclear localization sequence (NLS)
CCAAAGAAGAAGCGGAAGGTC
SEQ ID NO: 59
sgRNA scaffold sequence
GUUUUAGAGCUAGAAAUAGCAAGUUAAAAUAAGGCUAGUCCGUUAUCAACUUGAAAAAGUGGC
ACCGAGUCGGUGCUUUUUU
SEQ ID NO: 60
crRNA repeat sequence
GUUUUAGAGCUAUGCUGUUUUG
SEQ ID NO: 61
Native tracrRNA sequence
GGAACCAUUCAAAACAGCAUAGCAAGUUAAAAUAAGGCUAGUCCGUUAUCAACUUGAAAAAGU
GGCACCGAGUCGGUGCUUUUUU
SEQ ID NO: 62
E2A self-cleaving peptide
QCTNYALLKLAGDVESNPGP
SEQ ID NO: 63
T2A self-cleaving peptide
EGRGSLLTCGDVEENPGP
SEQ ID NO: 64
P2A self-cleaving peptide
ATNFSLLKQAGDVEENPGP
SEQ ID NO: 65
F2A self-cleaving peptide
VKQTLNFDLLKLAGDVESNPGP [symbol]
indicates data missing or illegible when filed

### VIII. References

1. Hsu P D, Lander E S, Zhang F. Development and applications of CRISPR-Cas9 for genome engineering. Cell. June 2014; 157(6): 1262-1278. doi:10.1016/j.cell.2014.05.010
2. Xiong X, Chen M, Lim W A, Zhao D, Qi L S. CRISPR/Cas9 for Human Genome Engineering and Disease Research. Annu Rev Genomics Hum Genet. 08 2016; 17:131-54. doi:10.1146/annurev-genom-083115-022258
3. Certo M T, Ryu B Y, Annis J E, et al. Tracking genome engineering outcome at individual DNA breakpoints. Nat Methods. July 2011; 8(8):671-6. doi10.1038/nmeth.1648
4. Yip B H. Recent Advances in CRISPR/Cas9 Delivery Strategies. Biomolecules. 05 2020; 10(6) doi10.3390/biom10060839
5. Lino C A, Harper J C, Carney J P, Timlin J A. Delivering CRISPR: a review of the challenges and approaches. Drug Deliv. November 2018; 25(1): 1234-1257, doi:10.1080/10717544.2018.1474964
6. Dever D P, Bak R O, Reinisch A, et al. CRISPR/Cas9 β-globin gene targeting in human haematopoietic stem cells. Nature. 11 2016; 539(7629):384-389. doi10.1038/nature20134
7. Sather B D, Romano Ibarra G S, Sommer K, et al. Efficient modification of CCR5 in primary human hematopoietic cells using a megaTAL nuclease and AAV donor template. Sci Transl Med. September 2015; 7(307): 307ra156. doi10.1126/scitranslmed.aac5530
8. Wang J, Exline C M, DeClercq J J, et al. Homology-driven genome editing in hematopoietic stem and progenitor cells using ZFN mRNA and AAV6 donors. Nat Biotechnol. December 2015; 33(12): 1256-1263. doi10.1038/nbt.3408
9. Simon A J, Ellington A D, Finkelstein I J. Retrons and their applications in genome engineering. Nucleic Acids Res. 12 2019; 47(21): 11007-11019. doi10.1093/nar/gkz865
10. Inouye S, Hsu M Y, Eagle S, Inouye M. Reverse transcriptase associated with the biosynthesis of the branched RNA-linked msDNA in Myxococcus xanthus. Cell. February 1989; 56(4): 709-17. doi10.1016/0092-8674(89)90593-x
11. Yee T, Furuichi T, Inouye S, Inouye M. Multicopy single-stranded DNA isolated from a gram-negative bacterium, Myxococcus xanthus. Cell. August 1984; 38(1):203-9. doi10.1016/0092-8674(84)90541-5
12. Shimamoto T, Hsu M Y, Inouye S, Inouye M. Reverse transcriptases from bacterial retrons require specific secondary structures at the 5′-end of the template for the cDNA priming reaction. J Biol Chem. February 1993; 268(4):2684-92.
13. Shimamoto T, Inouye M. Inouye S. The formation of the 2′,5′-phosphodiester linkage in the cDNA priming reaction by bacterial reverse transcriptase in a cell-free system. J Biol Chem. January 1995; 270(2):581-8. doi10.1074/jbc.270.2.581
14. Sharon E, Chen S A, Khosla N M, Smith J D, Pritchard J K, Fraser H B. Functional Genetic Variants Revealed by Massively Parallel Precise Genome Editing. Cell. 10 2018; 175(2):544-557.e16. doi10.1016/j.cell.2018.08.057
15. Schubert M G, Goodman D B, Wannier T M, et al. High throughput functional variant screens via in-vivo production of single-stranded DNA, bioRxiv. 2020:2020.03.05.975441. doi10.1101/2020.03.05.975441
16. Farzadfard F, Lu T K. Synthetic biology. Genomically encoded analog memory with precise in vivo DNA writing in living cell populations. Science. November 2014; 346(6211): 1256272. doi10.1126/science.1256272
17. Mirochnitchenko O, Inouye S, Inouye M. Production of single-stranded DNA in mammalian cells by means of a bacterial retron. J Biol Chem. January 1994; 269(4):2380-3.
18. Chu V T, Weber T, Wefers B, et al. Increasing the efficiency of homology-directed repair for CRISPR-Cas9-induced precise gene editing in mammalian cells. Nat Biotechnol. May 2015:33(5): 543-8. doi10.1038/nbt.3198
19. Gilbert L A, Horlbeck M A, Adamson B, et al. Genome-Scale CRISPR-Mediated Control of Gene Repression and Activation. Cell. October 2014; 159(3):647-61. doi:10.1016/j.cell.2014.09.029
20. Gray S J, Foti S B, Schwartz J W, et al. Optimizing promoters for recombinant adeno-associated virus-mediated gene expression in the peripheral and central nervous system using self-complementary vectors. Hum Gene Ther. September 2011; 22(9): 1143-53. doi10.1089/hum.2010.245
21. Ibrahimi A, Vande Velde G, Reumers V, et al. Highly efficient multicistronic lentiviral vectors with peptide 2A sequences. Hum Gene Ther. August 2009; 20(8):845-60. doi10.1089/hum.2008.188
22. Richardson C D, Ray G J, DeWitt M A, Curie G L, Corn J E. Enhancing homology-directed genome editing by catalytically active and inactive CRISPR-Cas9 using asymmetric donor DNA. Nat Biotechnol. March 2016; 34(3): 339-44. doi10.1038/nbt.3481
23. Millman A, Bernheim A, Stokar-Avihail A, et al. Bacterial Retrons Function In Anti-Phage Defense. Cell. December 2020; 183(6): 1551-1561.e12. doi10.1016/j.cell.2020.09.065
24. Gao L, Altae-Tran H, Böhning F, et al. Diverse enzymatic activities mediate antiviral immunity in prokaryotes. Science. 08 2020; 369(6507): 1077-1084. doi:10.1126/science.aba0372
25. Lee K, Mackley V A. Rao A, et al. Synthetically modified guide RNA and donor DNA engineering. Elife. 05 are versatile platform for CRISPR-Cas9 2017; 6 doi:10.7554/eLife.25312
26. Roy K R, Smith J D, Vonesch S C, et al. Multiplexed precision genome editing with trackable genomic barcodes in yeast. Nat Biotechnol. 07 2018; 36(6):512-520. doi:10.1038/nbt.4137
27. Zhao C, Liu F, Pyle A M. An ultraprocessive, accurate reverse transcriptase encoded by a metazoan group II intron. RNA. 02 2018; 24(2): 183-195. doi:10.1261/ma.063479.117
28. Wannier T M, Ciaccia P N. Ellington A D, et al. Recombincering and MAGE. Nature Reviews Methods Primers. 2021 Jan. 14 2021; 1(1): 7. doi:10.1038/s43586-020-00006-x
29. Wang C, Cheng J K W, Zhang Q, et al. Microbial single-strand annealing proteins enable CRISPR gene-editing tools with improved knock-in efficiencies and reduced off-target effects. Nucleic Acids Res. February 2021; doi:10.1093/nar/gkaa1264
30. Gaudelli N M, Komor A C, Rees H A, et al. Programmable base editing of A•T to G•C genomic DNA without DNA cleavage. Nature. 11 2017; 551(7681):464-471, in doi10.1038/nature24644
31. Rees H A, Liu D R. Base editing: precision chemistry on the genome and transcriptome of living cells. Nat Rev Genet. 12 2018; 19(12): 770-788. doi10.1038/s41576-018-0059-1
32. Komor A C, Kim Y B, Packer M S, Zuris J A, Liu D R. Programmable editing of a target base in genomic DNA without double-stranded DNA cleavage. Nature. 05 2016; 533(7603):420-4. doi10.1038/nature17946
33. Anzalone A V, Randolph P B, Davis J R, et al. Search-and-replace genome editing without double-strand breaks or donor DNA. Nature. 12 2019; 576(7785): 149-157. doi10.1038/s41586-019-1711-4
34. Rothemund P W. Folding DNA to create nanoscale shapes and patterns. Nature. March 2006; 440(7082):297-302. doi10.1038/nature04586
35. Datta H J, Glazer P M. Intracellular generation of single-stranded DNA for chromosomal triplex formation and induced recombination. Nucleic Acids Res. December 2001; 29(24):5140-7. doi10.1093/nar/29.24.5140
36. Keefe A D, Pai S, Ellington A. Aptamers as therapeutics. Nat Rev Drug Discov. 07 2010; 9(7):537-50. doi10.1038/nrd3141
37. Lee J F, Stovall G M, Ellington A D. Aptamer therapeutics advance. Curr Opin Chem Biol. June 2006; 10(3):282-9. doi10.1016/j.cbpa.2006.03.015
38. Lim D, Maas W K. Reverse transcriptase-dependent synthesis of a covalently linked, branched DNA-RNA compound in E. coli B. Cell 1989; 56:891-904. DOI: 10.1016/0092-8674(89)90693-4.
39. Inouye S, Herzer P J, Inouye M. Two independent retrons with highly diverse reverse transcriptases in Myxococcus xanthus. Proc Natl Acad Sci USA 1990; 87:942-945. DOI: 10.1073/pnas.87.3.942.
40. Sun J, Inouye M, Inouye S. Association of a retroelement with a P4-like cryptic prophage (retronphage phi R73) integrated into the selenocystyl tRNA gene of Escherichia coli. J Bacteriol 1991; 173:4171-4181. DOI: 10.1128/jb.173.13.4171-4181.1991.
41. Herzer P J, Inouye S, Inouye M. Retron-Ec107 is inserted into the Escherichia coli genome by replacing a palindromic 34 bp intergenic sequence. Mol Microbiol 1992; 6:345-354. DOI: 10.1111/j.1365-2958.1992.tb01477.x.
42. Ahmed A M, Shimamoto T. msDNA-St85, a multicopy single-stranded DNA isolated from Salmonella enterica serovar Typhimurium LT2 with the genomic analysis of its retron. FEMS Microbiol Lett 2003; 224:291-297. DOI: 10.1016/S0378-1097(03)00450-6.
43. Lampson B C, Sun J, Hsu M Y, Vallejo-Ramirez J, Inouye S, Inouye M. Reverse transcriptase in a clinical strain of Escherichia coli: production of branched RNA-linked msDNA. Science 1989; 243:1033-1038. DOI: 10.1126/science.2466332.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1-11. (canceled)

12. A vector comprising:
a guide RNA (gRNA)-retron cassette for use in genomic editing in a mammalian cell comprising:
(a) a gRNA coding region, wherein the target sequence of the gRNA is within a mammalian genetic locus; and
(b) a retron region comprising:
(i) an msr locus;
(ii) a first inverted repeat sequence:
(lii) an msd locus;
(iv) a donor DNA template region located within the msd locus, wherein the donor DNA template comprises homology to one or more sequences within the mammalian genetic locus; and
(v) a second inverted repeat sequence; and
a RNA polymerase III (Pol III) promoter operably linked to the cassette,
wherein the gRNA coding region is upstream of the retron region in the cassette such that transcription of the cassette results in a transcript in which the gRNA is 5′ of the RNA transcribed from the retron region, and
wherein the msd locus comprises one or more sequence modifications to avoid pre-mature Pol III termination, wherein the sequence modification is to modify the sequence of TTTT to TTTC or TTTA in a stem region of the msd locus.

13. The vector of claim 12, wherein the msd locus further comprises a modification of a corresponding sequence in the opposite strand of the stem region for maintaining secondary structure.

14. The vector of claim 13, wherein the modification of the corresponding sequence comprises modifying GGAAA to GGGAA or GAAAA to GGAAA.

15. A vector comprising:
a guide RNA (gRNA)-retron cassette for use in genomic editing in a mammalian cell comprising:
(a) a gRNA coding region, wherein the target sequence of the gRNA is within a mammalian genetic locus; and
(b) a retron region comprising:
(i) an msr locus;
(ii) a first inverted repeat sequence;
(lii) an msd locus;
(iv) a donor DNA template region located within the msd locus, wherein the donor DNA template comprises homology to one or more sequences within the mammalian genetic locus; and
(v) a second inverted repeat sequence; and
a RNA polymerase III (Pol III) promoter operably linked to the cassette,
wherein the gRNA coding region is upstream of the retron region in the cassette such that transcription of the cassette results in a transcript in which the gRNA is 5′ of the RNA transcribed from the retron region,
wherein the msd locus comprises one or more sequence modifications to avoid pre-mature Pol III termination, wherein the sequence modification comprises modifying TTTT to TTTC or TTTA in a stem region of the msd locus, and modifying TTTTTT to TTTCTT downstream of the stem region.

16-58. (canceled)


SEQUENCE LISTING (65 sequences)

SEQ ID NO: 1 - DNA, 200 bp, synthetic construct
  misc_feature 1..200: note = Description of sequence: OBZ206: qPCR standard template
  misc_feature 1..200: note = Single-stranded oligodeoxynucleotides (ssODNs) sequence
  source 1..200: mol_type = other DNA
gacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccg

SEQ ID NO: 2 - DNA, 19 bp, synthetic construct
  misc_feature 1..19: note = Description of sequence: qPCR primer-Forward primer: OBZ152
  source 1..19: mol_type = other DNA
agctgaagggcatcgactt

SEQ ID NO: 3 - DNA, 20 bp, synthetic construct
  misc_feature 1..20: note = Description of sequence: qPCR primer-Reverse primer: OBZ153
  source 1..20: mol_type = other DNA
ggatcttgaagttggccttg

SEQ ID NO: 4 - DNA, 208 bp, synthetic construct
  misc_feature 1..208: note = Description of sequence: Donor sequence (Donor sequence used in qPCR
  source 1..208: mol_type = other DNA
tcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcaccgccgacaagcagaagaacggcatcaaggccaacttcaagatccg

SEQ ID NO: 5 - DNA, 128 bp, synthetic construct
  misc_feature 1..128: note = Description of sequence: Ec86 msr-msd sequence
  source 1..128: mol_type = other DNA
atgcgcacccttagcgagaggtttatcattaaggtcaacctctggatgttgtttcggcatcctgcattgaatctgagttactgtctgttttcctaggaaacccgtttcttctgacgtaagggtgcgca

SEQ ID NO: 6 - DNA, 128 bp, synthetic construct
  misc_feature 1..128: note = Description of sequence: "Ec86 msr-msd sequence with ""TTTT && TTTc"" modification"
  source 1..128: mol_type = other DNA
atgcgcacccttagcgagaggtttatcattaaggtcaacctctggatgttgtttcggcatcctgcattgaatctgagttactgtctgtttccctaggaaacccgtttcttctgacgtaagggtgcgca

SEQ ID NO: 7 - DNA, 128 bp, synthetic construct
  misc_feature 1..128: note = Description of sequence: "Ec86 msr-msd sequence with ""TTTT && TTTc"" and ""GGAAA && GGgAA"" modifications"
  source 1..128: mol_type = other DNA
atgcgcacccttagcgagaggtttatcattaaggtcaacctctggatgttgtttcggcatcctgcattgaatctgagttactgtctgtttccctagggaacccgtttcttctgacgtaagggtgcgca

SEQ ID NO: 8 - DNA, 128 bp, synthetic construct; identical to SEQ ID NO: 7
  misc_feature 1..128: note = Description of sequence: "Ec86 msr-msd sequence with ""TTTT && TTTc"" ""GGAAA && GGgAA"" and ""TTTTTT && TTTcTT"" modifications"
  source 1..128: mol_type = other DNA

SEQ ID NO: 9 - protein, 320 aa, synthetic construct
  REGION 1..320: note = Description of sequence: Ec86 RT
MKSAEYLNTFRLRNLGLPVMNNLHDMSKATRISVETLRLLIYTADFRYRIYTVEKKGPEKRMRTIYQPSRELKALQGWVLRNILDKLSSSPFSIGFEKHQSILNNATPHIGANFILNIDLEDFFPSLTANKVFGVFHSLGYNRLISSVLTKICCYKNLLPQGAPSSPKLANLICSKLDYRIQGYAGSRGLIYTRYADDLTLSAQSMKKVVKARDFLFSIIPSEGLVINSKKTCISGPRSQRKVTGLVISQEKVGIGREKYKEIRAKIHHIFCGKSSEIEHVRGWLSFILSVDSKSHRRLITYISKLEKKYGKNPLNKAKT

SEQ ID NO: 10 - DNA, 194 bp, synthetic construct
  misc_feature 1..194: note = Description of sequence: Ec107 msr-msd sequence
  source 1..194: mol_type = other DNA
cgccagcagtggcaatagcgtttccggccttttgtgccgggagggtcggcgagtcgctgacttaacgccagtagtatgtccatatacccaaagtcgcttcattgtacctgagtacgcttcgcgtacgtcgcgctgacgcgctcagtacagttacgcgccttcgggatggtttaatggtattgccgctgttggcg

SEQ ID NO: 11 - DNA, 194 bp, synthetic construct
  misc_feature 1..194: note = Description of sequence: "Ec107 msr-msd sequence with ""TTTT && TTTa"" modification"
  source 1..194: mol_type = other DNA
cgccagcagtggcaatagcgtttccggcctttagtgccgggagggtcggcgagtcgctgacttaacgccagtagtatgtccatatacccaaagtcgcttcattgtacctgagtacgcttcgcgtacgtcgcgctgacgcgctcagtacagttacgcgccttcgggatggtttaatggtattgccgctgttggcg

SEQ ID NO: 12 - protein, 319 aa, synthetic construct
  REGION 1..319: note = Description of sequence: Ec107 RT
MDATRTTLLALDLFGSPGWSADKEIQRLHALSNHAGRHYRRIILSKRHGGQRLVLAPDYLLKTVQRNILKNVLSQFPLSPFATAYRPGCPIVSNAQPHCQQPQILKLDIENFFDSISWLQVWRVFRQAQLPRNVVTMLTWICCYNDALPQGAPTSPAISNLVMRRFDERIGEWCQARGITYTRYCDDMTFSGHFNARQVKNKVCGLLAELGLSLNKRKGCLIAACKRQQVTGIVVNHKPQLAREARRALRQEVHLCQKYGVISHLSHRGELDPSGDLHAQATAYLYALQGRINWLLQINPEDEAFQQARESVKRMLVAW

SEQ ID NO: 13 - DNA, 140 bp, synthetic construct
  misc_feature 1..140: note = Description of sequence: Mx65 msr-msd sequence
  source 1..140: mol_type = other DNA
ggatccgggctcgcagatgagccatgagtaccgcggtgtttcgccgcgggggtgttctgtccccatctcttcgccagggtcccagcgtacgcaacgcagggagccccgggtccaacgcctcgcaggtcgtcccctggcct

SEQ ID NO: 14 - protein, 427 aa, synthetic construct
  REGION 1..427: note = Description of sequence: Mx65 RT
MSWFDTTLSRLKGLFSRPVTRSTTGLDVPLDAHGRPQDVVTETVSTSGPLKPGHLRQVRRDARLLPKGVRRYTPGRKKWMEAAEARRLFSATLRTRNRNLRDLLPDEAQLARYGLPVWRTEEDVAAALGVSVGVLRHYSIHRPRERVRHYVTFAVPKRSGGVRLLHAPKRRLKALQRRMLALLVSKLPVSPQAHGFVPGRSIKTGAAPHVGRRVVLKLDLKDFFPSVTFARVRGLLIALGYGYPVAATLAVLMTESERQPVELEGILFHVPVGPRVCVQGAPTSPALCNAVLLRLDRRLAGLARRYGYTYTRYADDLTFSGDDVTALERVRALAARYVQEEGFEVNREKTRVQRRGGAQRVTGVTVNTTLGLSREERPRLRAMLHQEARSEDVEAHRAHLDGLLAYVKMLNPEQAERLARRRKPRGT

SEQ ID NO: 15 - DNA, 236 bp, synthetic construct
  misc_feature 1..236: note = Description of sequence: Mx162 msr-msd sequence
  source 1..236: mol_type = other DNA
gcgcgagcagccgagagaggtccggagtgcatcagcctgagcgcctcgagcggcggagcggcgttgcgccgctccggttggaatgcaggacactctccgcaaggtagcctgttcttggctctctccctcctaggcactacggccagggtgggtagcggagccaacgacgcgaccgccgtttacccaccccggccgtagtgcctaggaggggagagccggtgaggctaccgtgcccc

SEQ ID NO: 16 - protein, 485 aa, synthetic construct
  REGION 1..485: note = Description of sequence: Mx162 RT
MTARLDPFVPAASPQAVPTPELTAPSSDAAAKREARRLAHEALLVRAKAIDEAGGADDWVQAQLVSKGLAVEDLDFSSASEKDKKAWKEKKKAEATERRALKRQAHEAWKATHVGHLGAGVHWAEDRLADAFDVPHREERARANGLTELDSAEALAKALGLSVSKLRWFAFHREVDTATHYVSWTIPKRDGSKRTITSPKPELKAAQRWVLSNVVERLPVHGAAHGFVAGRSILTNALAHQGADVVVKVDLKDFFPSVTWRRVKGLLRKGGLREGTSTLLSLLSTEAPREAVQFRGKLLHVAKGPRALPQGAPTSPGITNALCLKLDKRLSALAKRLGFTYTRYADDLTFSWTKAKQPKPRRTQRPPVAVLLSRVQEVVEAEGFRVHPDKTRVARKGTRQRVTGLVVNAAGKDAPAARVPRDVVRQLRAAIHNRKKGKPGREGESLEQLKGMAAFIHMTDPAKGRAFLAQLTELESTASAAPQAE

SEQ ID NO: 17 - DNA, 158 bp, synthetic construct
  misc_feature 1..158: note = Description of sequence: St85 msr-msd sequence
  source 1..158: mol_type = other DNA
actctttagcgttaggctttgatttatagccttgtcgagcgtttcgccagacactaacttattgagtacttttagggttgcgctagaaagttttctaccgatcctagaagtctctaggatcggtagaaaactttctagcgcctcctctagtaaagagt

SEQ ID NO: 18 - DNA, 158 bp, synthetic construct
  misc_feature 1..158: note = Description of sequence: "St85 msr-msd sequence with ""TTTT && TTTc"" modification"
  source 1..158: mol_type = other DNA
actctttagcgttaggctttgatttatagccttgtcgagcgtttcgccagacactaacttattgagtactttcagggttgcgctagaaagttttctaccgatcctagaagtctctaggatcggtagaaaactttctagcgcctcctctagtaaagagt

SEQ ID NO: 19 - DNA, 158 bp, synthetic construct
  misc_feature 1..158: note = Description of sequence: "St85 msr-msd sequence with ""TTTT && TTTc"" and ""GAAAA && GgAAA"" modifications"
  source 1..158: mol_type = other DNA
actctttagcgttaggctttgatttatagccttgtcgagcgtttcgccagacactaacttattgagtactttcagggttgcgctagaaagttttctaccgatcctagaagtctctaggatcggtaggaaactttctagcgcctcctctagtaaagagt

SEQ ID NO: 20 - protein, 311 aa, synthetic construct
  REGION 1..311: note = Description of sequence: St85 RT
MDILQHISDLLLTKKSEIISFSLTAPYRYKIYKIAKRNSDKKRTIAHPSKELKFIQREITEYLTDKLPVHECAFAYKKGSSIKTNAQVHLHTKYLLKMDFENFFPSITPRLFFSKLRLANIDLTADDKVLLENILFFKSKRNSNLRLSIGAPSSPLISNFVMYFWDIEVQEICSKIGVNYTRYADDLTFSTNNKDVLFDIPDMLENVLPKYSLGRIRINHEKTVFSSKGHNRHVTGITLTNDNKLSIGRERKRKISAMIHHFINGKLSTDECNKLVGLLAFAKNIEPSFYKSMVIKYGSDNIYKLQKQKDK

SEQ ID NO: 21 - DNA, 132 bp, synthetic construct
  misc_feature 1..132: note = Description of sequence: Ec67 msr-msd sequence
  source 1..132: mol_type = other DNA
cacgcatgtaggcagatttgttggttgtgaatcgcaaccagtggccttaatggcaggaggaatcgcctccctaaaatccttgattcagagctatacggcaggtgtgctgtgcgaaggagtgcctgcatgcgt

SEQ ID NO: 22 - protein, 586 aa, synthetic construct
  REGION 1..586: note = Description of sequence: Ec67 RT
MTKTSKLDALRAATSREDLAKILDIKLVFLTNVLYRIGSDNQYTQFTIPKKGKGVRTISAPTDRLKDIQRRICDLLSDCRDEIFAIRKISNNYSFGFERGKSIILNAYKHRGKQIILNIDLKDFFESFNFGRVRGYFLSNQDFLLNPVVATTLAKAACYNGTLPQGSPCSPIISNLICNIMDMRLAKLAKKYGCTYSRYADDITISTNKNTFPLEMATVQPEGVVLGKVLVKEIENSGFEINDSKTRLTYKTSRQEVTGLTVNRIVNIDRCYYKKTRALAHALYRTGEYKVPDENGVLVSGGLDKLEGMFGFIDQVDKFNNIKKKLNKQPDRYVLTNATLHGFKLKLNAREKAYSKFIYYKFFHGNTCPTIITEGKTDRIYLKAALHSLETSYPELFREKTDSKKKEINLNIFKSNEKTKYFLDLSGGTADLKKFVERYKNNYASYYGSVPKQPVIMVLDNDTGPSDLLNFLRNKVKSCPDDVTEMRKMKYIHVFYNLYIVLTPLSPSGEQTSMEDLFPKDILDIKIDGKKFNKNNDGDSKTEYGKHIFSMRVVRDKKRKIDFKAFCCIFDAIKDIKEHYKLMLNS

SEQ ID NO: 23 - DNA, 280 bp, synthetic construct
  misc_feature 1..280: note = Description of sequence: Sa163 msr-msd sequence
  source 1..280: mol_type = other DNA
gtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgacgcaaccgccgtttccccggccggagaggtactcaccggaggggagagccggtgaggctaccgtgccccaggtgagaaggtggtgccttcgggcctccctcgaccgctcgcgc

SEQ ID NO: 24 - protein, 480 aa, synthetic construct
  REGION 1..480: note = Description of sequence: Sa163 RT
MTAKLESHVPAAPPVSAEAPAPTRPDAAKQEARRAHHEALRLRWKAIEEAGGTDAWVRQQLVAKGVAAEEVDFESLSDKQKAAWKEKKKAEATERRAQKRLAWEAWKATHIHHLGVGVHWDEAGGPDKFDVAGREERAKANGLPEGLDSVEALAKALGISVSRLRWFSFHREVDTGTHYQTWEIPKRDGGKRTLTAPKRELKAVQRWVLANVVERLPVHGAAHGFVAGRSILTNALAHQGADVVVKVDMKDFFPSVTWPRVKGLLRKGGLPENLATLLALLSTEAPREVVRFRGETLYVAKGPRALPQGAPTSPALTNALCLRLDKRLSALSKRLGFTYTRYADDLTFSWRRAKKSRQKELPLADAPVALLLARVKGVLEAEGFTLHPDKTRVQRKGSRQRVTGLVVNEAPEGVPGARVPRDVVRRLRAAIHNREQGKPGPTGETLEQLKGLAAFLHMTDAEKGRAFLRRLEALEKRQTA

SEQ ID NO: 25 - DNA, 113 bp, synthetic construct
  misc_feature 1..113: note = Description of sequence: Ec73 msr-msd sequence
  source 1..113: mol_type = other DNA
agagccaaacctagcatttatgggttaatagcccatcgggccatgagtcatggtttcgcctagtattttagctatgcccgtcgttatcgacgtgctcaagtaggtttggctct

SEQ ID NO: 26 - protein, 586 aa, synthetic construct; identical to SEQ ID NO: 22
  REGION 1..586: note = Description of sequence: Ec73 RT

SEQ ID NO: 27 - DNA, 2946 bp, synthetic construct
  misc_feature 1..2946: note = Description of sequence: RT-EcoI/Ec86 in pTwist CMV:
  source 1..2946: mol_type = other DNA
atgtacccatacgacgtcccagactacgctcctcccaagaagaaaaggaaagttaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaaggactataaagatgacgatgacaaggactacaaggatgatgacgataaagattataaagacgacgatgataagaaaggtccgggaagcggagctactaacttcagcctgctgaagcaggctggagacgtggaggagaaccctggacctccggtcgccaccagcgagctgattaaggagaacatgcacatgaagctgtacatggagggcaccgtggacaaccatcacttcaagtgcacatccgagggcgaaggcaagccctacgagggcacccagaccatgagaatcaaggtggtcgagggcggccctctccccttcgccttcgacatcctggctactagcttcctctacggcagcaagaccttcatcaaccacacccagggcatccccgacttcttcaagcagtccttccctgagggcttcacatgggagagagtcaccacatacgaagacgggggcgtgctgaccgctacccaggacaccagcctccaggacggctgcctcatctacaacgtcaagatcagaggggtgaacttcacatccaacggccctgtgatgcagaagaaaacactcggctgggaggccttcaccgagacgctgtaccccgctgacggcggcctggaaggcagaaacgacatggccctgaagctcgtgggcgggagccatctgatcgcaaacatcaagaccacatatagatccaagaaacccgctaagaacctcaagatgcctggcgtctactatgtggactacagactggaaagaatcaaggaggccaacaacgagacctacgtcgagcagcacgaggtggcagtggccagatactgcgacctccctagcaaactggggcacaagcttaattaattaagaattcgtcgagggacctaataacttcgtatagcatacattatacgaagttatacatgtttaagggttccggttccactaggtacaattcgatatcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagcggccgcgaggtgcttgtagataacctccacgatggtgcaccttgggcaacacaaaagtggcaaatcatctacaatgcgcacccttagcgagaggtttatcattaaggtcaacctctggatgttgtttcggcatcctgcattgaatctgagttactgtctgttttcctggccatgctggccagcggataacaagtttaaacacggccacagtggccaggaaacccgtttcttctgacgtaagggtgcgcagacactgagtgagaaacgtccccgtcgtagtgtcggtaatgcgttgtttcaacgtagccaattctcacggcgcgcc

SEQ ID NO: 28 - DNA, 1932 bp, synthetic construct
  misc_feature 1..1932: note = Description of sequence: RT-EcoII/Ec67 in pTwist EF1 Alpha Puro:
  source 1..1932: mol_type = other DNA
atgtacccatacgacgtcccagactacgctcctcccaagaagaaaaggaaagttatgactaaaacctctaaactcgacgcactcagagcagcaacatcccgcgaagacctcgccaaaattttggatattaaacttgtgttccttacaaatgtcctgtatcgcatagggtcagataatcaatacacacagtttacgatcccgaagaagggtaagggcgtgcgaaccatatcagccccgaccgaccggctgaaggacattcagcggagaatctgtgatttgttgagcgactgtcgggatgaaatctttgcgattcggaagataagtaataattattctttcggatttgaaagggggaaatccataattctcaatgcctataaacaccgcggtaagcagattatccttaacattgatcttaaagatttctttgagtccttcaacttcggtagggtacgaggttactttctttctaaccaggattttttgctcaatcctgttgtggcgactacactggcaaaagcggcatgctataatgggaccctgcctcagggcagcccttgcagcccaataatatcaaaccttatctgcaacatcatggatatgcgcttggcaaagctggccaagaaatacggatgtacatacagtcggtacgccgatgatatcaccatctctacaaataagaatacttttcctctcgaaatggccacagttcaaccggagggcgtcgtcctggggaaggtgctcgtaaaagaaatcgaaaactctggtttcgagatcaatgattctaagacgagactgacttataaaactagtagacaggaggttacggggctgaccgtaaaccgcatagtaaatatcgatcgatgctactataagaaaacaagggcgcttgcacacgcactttatcggacaggtgaatataaggtccctgacgagaacggcgtcctggtttcaggcgggctggataagctcgaaggaatgtttggctttatcgatcaagtcgacaaattcaacaatataaagaaaaagcttaacaaacagccagataggtacgtcttgacaaacgccacactgcatgggttcaagctgaaactcaacgctcgcgagaaggcgtactcaaagttcatttactacaagtttttccatggtaatacgtgtcctactatcataactgaggggaagacagaccgcatatatctgaaggcagctctccacagtctcgagacctcatatcccgaactttttcgcgagaagacggattcaaaaaagaaggaaataaatttgaatatcttcaaatcaaacgaaaagacaaaatatttcctcgacctctctggcgggaccgcggatcttaagaaattcgtagagcggtacaagaataattacgcgtcctattatggatccgttcccaagcagccagtcataatggttctcgataacgacactgggccgagcgatctcctcaattttctgagaaacaaagtgaaatcctgtcctgatgatgtgactgaaatgcgcaaaatgaagtatatacacgtcttctacaatctctacatcgtgctgacgccattgagcccctcaggggagcaaacgtcaatggaggacctcttccccaaggatattctcgacatcaagatagatggaaaaaagttcaacaaaaacaatgatggggattcaaagacggaatatggaaaacacatcttctcaatgcgagtcgtacgcgataaaaaaaggaagatcgacttcaaagctttttgttgcattttcgacgcaatcaaggatataaaagaacattataagcttatgttgaacagcaaaaggccggcggccacgaaaaaggccggccaggcaaaaaagaaaaaggactataaagatgacgatgacaaggactacaaggatgatgacgataaagattataaagacgacgatgataag

SEQ ID NO: 29 - DNA, 1165 bp, synthetic construct
  misc_feature 1..1165: note = Description of sequence: RT-EcoIII/Ec73
  source 1..1165: mol_type = other DNA
gaagtgccattccgcctgacctatgtacccatacgacgtcccagactacgctcctcccaagaagaaaaggaaagttatgcgaatatactcccttatagacagtcagaccctcatgaccaaggggtttgcctcagaggtcatgcggtcaccagaacccccgaagaagtgggacatagcaaaaaagaagggtggtatgcggacaatttatcacccatcatctaaagttaaacttatccagtactggctcatgaacaacgtatttagtaagctgccaatgcacaacgcggcttatgcttttgttaagaatcggtcaatcaagtctaacgcgcttctccacgcagagtcaaaaaacaaatactacgttaagatcgatctcaaagacttctttccctcaattaagttcacggattttgagtacgctttcactaggtatcgcgacagaatcgaatttacaaccgagtatgacaaagaattgctgcagttgatcaagactatctgttttatctctgacagcacgctcccgataggtttcccaaccagcccactcattgcgaatttcgtggcgagggagctggacgagaaacttacccagaaactcaatgctatagataaacttaatgcgacatatacccgctatgcagacgatattattgtctcaactaatatgaaaggagcgagcaaattgatccttgactgcttcaagcgaactatgaaggagatcgggccggacttcaagatcaatatcaaaaagttcaaaatatgtagcgccagcggcgggtcaatagtggtcacggggctgaaagtgtgccacgattttcacattacgcttcatcggagtatgaaagataaaataaggttgcacttgagcttgctctctaagggaattttgaaagacgaagaccataacaaactttcaggttatattgcatacgctaaagacatagacccccacttttataccaaacttaaccggaaatacttccaagaaattaagtggatccaaaacctgcataataaagttgaaaaaaggccggcggccacgaaaaaggccggccaggcaaaaaagaaaaaggactataaagatgacgatgacaaggactacaaggatgatgacgataaagattataaagacgacgatgataagaggctaggtggaggctcagtg

SEQ ID NO: 30 - DNA, 1174 bp, synthetic construct
  misc_feature 1..1174: note = Description of sequence: RT-EcoV/Ec107
  source 1..1174: mol_type = other DNA
gaagtgccattccgcctgacctatgtacccatacgacgtcccagactacgctcctcccaagaagaaaaggaaagttatggacgccacccgaaccactcttcttgcgttggatctcttcgggtcaccagggtggtccgccgacaaggagatacagcgccttcacgctctgagcaatcatgctggccgccattataggaggatcattctttctaaacggcacggaggccagagactcgtcttggcgccggattacttgttgaaaactgtccaacggaatatactgaaaaacgtattgtcccaatttccgttgtcaccctttgccactgcctatcgaccaggatgccctatcgtatctaacgcgcaaccacactgccagcaaccacagatactgaaattggacattgaaaatttctttgactctatcagttggctccaagtatggcgcgttttcagacaggctcagctccctcgaaatgttgtgacaatgcttacctggatttgttgttacaacgatgccctcccgcaaggtgcgccaacatcccctgcgatatctaatctcgtcatgaggcggttcgatgagagaatcggggaatggtgccaggctcgcggaatcacatacacgcggtattgtgacgacatgacattttccggccatttcaacgcccgacaagtaaagaacaaagtgtgtggccttctcgcagagcttgggttgtctctgaataagcgaaaagggtgccttattgcggcttgtaagcggcagcaagtgacaggtattgtggtgaaccataaaccgcaacttgcaagagaggcccgccgggctctccgacaggaggtgcatctctgtcagaaatatggggtcatctcacacttgagtcacaggggtgaactcgacccgagtggagacctgcacgcccaggcgacggcctacttgtacgcgcttcaaggaaggataaactggctccttcaaatcaatcctgaagacgaagcgttccagcaggccagggagtctgttaaacggatgctcgtagcatggaaaaggccggcggccacgaaaaaggccggccaggcaaaaaagaaaaaggactataaagatgacgatgacaaggactacaaggatgatgacgataaagattataaagacgacgatgataagaggctaggtggaggctcagtg

SEQ ID NO: 31 - DNA, 1498 bp, synthetic construct
  misc_feature 1..1498: note = Description of sequence: RT-MaxII/Mx65
  source 1..1498: mol_type = other DNA
gaagtgccattccgcctgacctatgtacccatacgacgtcccagactacgctcctcccaagaagaaaaggaaagttatgagttggttcgatacaactctgtctcggttgaaagggcttttttcaaggccggttactcggagcacaacaggactggatgtcccattggacgcgcacgggaggccgcaggacgttgttacagaaactgtcagtaccagtggtcctctcaagccaggtcatcttcgccaagtgagacgagatgctcggcttctcccgaaaggtgttcgacgctacacccccggccgaaagaaatggatggaggccgcggaggccaggcgactcttttctgccacgctgcgaacgagaaaccgaaatctgagagatctgttgccagacgaagcacaacttgctcgctatggtttgccagtctggcgcaccgaagaagacgtggcagcggccctcggagtatcagttggcgtccttaggcattactctatacataggccgcgcgagcgggtgaggcactacgtaacgttcgcggtcccaaaacgaagcggtggcgtcaggttgctccatgcacctaaacggcggcttaaagcactccagcgccggatgctggcacttctggtaagcaaactccctgtgtccccacaggctcatggtttcgtgcccggaagaagtataaagactggggcggcccctcatgtaggtaggcgcgtagtcctcaagctcgatttgaaggacttctttccgtctgttacgtttgcgcgggttagaggcctgcttattgcactcggctatggttatcctgttgctgctacactcgcagtccttatgacggaatcagagcgccaacctgtcgagttggaaggaatcctgtttcatgtcccagttggcccaagagtttgcgtacagggggcgcctacttccccagcactttgcaacgctgttcttcttcgattggacaggagacttgccggccttgcaagacgatatggttacacgtacacacggtatgcggacgacttgacattctcaggagacgacgtcacggcgttggagcgagttagagctcttgcggcaagatatgtccaagaagaaggtttcgaggtgaacagagagaaaactcgcgtgcagaggcgaggcggagcccagcgagtgacgggcgtgaccgtgaacacgacactgggactgtcaagggaagaacgccctaggttgcgagcaatgctccaccaggaggcacgatctgaagatgtggaagcacaccgagcccatctggatggcttgttggcctacgtcaaaatgctgaaccccgaacaagccgaacggttggcccgacgccgcaagccgcgggggaccaaaaggccggcggccacgaaaaaggccggccaggcaaaaaagaaaaaggactataaagatgacgatgacaaggactacaaggatgatgacgataaagattataaagacgacgatgataagaggctaggtggaggctcagtg

SEQ ID NO: 32 - DNA, 1672 bp, synthetic construct
  misc_feature 1..1672: note = Description of sequence: RT-MxaI/Mx162
  source 1..1672: mol_type = other DNA
gaagtgccattccgcctgacctatgtacccatacgacgtcccagactacgctcctcccaagaagaaaaggaaagttatgacagcaaggttggacccctttgtcccagcagcttcaccccaggcagtgccaaccccagaactgacggcgcccagctccgatgcggctgcaaaacgagaggcacggcgcctggcgcatgaagcgttgttggtaagggcgaaagctatcgatgaagctgggggtgccgacgattgggttcaggcccaactggtgtcaaaaggattggcggtggaagatctcgacttctcttctgcctcagagaaagataaaaaggcatggaaggaaaagaagaaagcggaggcaacagaaaggcgggcccttaagaggcaagcccatgaggcgtggaaagcaacccatgttggacacttgggcgcgggagtccactgggccgaagacagactcgcggatgccttcgacgtaccgcatagggaagaacgggcacgggcgaatgggctgaccgagctggacagtgcggaagcgttggcaaaagcccttggcttgtcagtgtcaaaactccgatggtttgcattccatagagaggtagatacagccactcattatgtatcttggacgatcccaaagagggacggctccaagcgaacgataacatcccccaagcccgagttgaaagccgctcaaaggtgggttctctcaaatgtggtggaaagactcccggtccatggtgctgctcacgggtttgtcgctggtcgcagcatactcactaacgctcttgctcatcagggagcggatgtagtcgtaaaagttgaccttaaagacttttttcctagtgtgacctggcgcagggtaaagggcctcttgaggaaaggtggactcagagagggcacaagtacgttgcttagtcttctttctaccgaagcacctcgcgaggctgtccaatttcgaggcaaattgctccatgttgcaaagggtccacgggcgcttccacagggagcccctacctcaccggggatcaccaacgcactctgtctcaagcttgataagcgcctttcagccctcgccaagaggttgggttttacttacacgcgctacgcggacgatttgaccttttcttggactaaggcgaagcagccaaagccacgacggacacaacggcctccagtcgcggttctcttgtctcgcgtccaagaagtggttgaggccgaaggctttagggtccatcctgacaagacccgagttgcccggaagggaacgcgacaacgcgttacggggttggtggtaaatgcggcggggaaagacgccccagccgctcgggtcccccgcgacgtggtaaggcagcttagggccgcgattcataaccgaaagaagggcaagcccggacgggaaggggagtctctggaacaattgaagggcatggctgcatttattcacatgacagaccccgcaaaaggtcgggcatttctggcacagcttacggagctggaaagtactgcgtccgccgcacctcaagcagaaaaaaggccggcggccacgaaaaaggccggccaggcaaaaaagaaaaaggactataaagatgacgatgacaaggactacaaggatgatgacgataaagattataaagacgacgatgataagaggctaggtggaggctcagtg

SEQ ID NO: 33 - DNA, 1150 bp, synthetic construct
  misc_feature 1..1150: note = Description of sequence: RT-SenII/St85
  source 1..1150: mol_type = other DNA
gaagtgccattccgcctgacctatgtacccatacgacgtcccagactacgctcctcccaagaagaaaaggaaagttatggatattctgcaacacatatccgacctgctgctgacaaagaagtcagaaattatttcttttagtctcaccgcaccttatcggtacaaaatttataagatcgctaaaagaaactccgacaagaaaagaaccatcgcccacccgagtaaagaacttaaattcatacagagagagattactgagtacctgacagataagctgcccgtgcacgaatgcgcctttgcttacaaaaagggctctagtataaaaacaaacgcacaagttcatctccatacgaaatatctgttgaaaatggattttgaaaacttttttccctccataacgccgcggctcttcttctcaaaactgcgcttggctaacatcgacttgactgctgatgataaggttttgttggagaatatcttgttttttaaatccaaacgcaacagtaatctgcgcttgagtatcggtgcgccgtcttctccattgatttccaacttcgttatgtacttctgggacattgaggtgcaagagatatgctccaagatcggggttaattatacccgctacgcagatgatttgacctttagcacaaataataaagacgtgctgtttgatatacctgatatgttggaaaacgttctgccgaagtatagtttggggcggatacggattaaccacgaaaagactgtgttctctagcaagggccacaaccgacatgtaaccgggataactcttactaatgataacaagctgtctatcggtcgggagcggaaaaggaagatcagcgcgatgatccaccacttcatcaatggaaagctgtcaacagatgagtgcaacaaattggtgggcctcctggccttcgccaaaaatatagagccatccttttacaaaagcatggttattaagtatgggagtgataacatttataaactccagaagcagaaagacaagaaaaggccggcggccacgaaaaaggccggccaggcaaaaaagaaaaaggactataaagatgacgatgacaaggactacaaggatgatgacgataaagattataaagacgacgatgataagaggctaggtggaggctcagtg

SEQ ID NO: 34 - DNA, 1614 bp, synthetic construct
  misc_feature 1..1614: note = Description of sequence: RT-SauI_Sa163
  source 1..1614: mol_type = other DNA
atgtacccatacgacgtcccagactacgctcctcccaagaagaaaaggaaagttatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaaggactataaagatgacgatgacaaggactacaaggatgatgacgataaagattataaagacgacgatgataag

SEQ ID NO: 35 - DNA, 9277 bp, synthetic construct
  misc_feature 1..9277: note = Description of sequence: pBZ186-pU6-(BbsI)_CBh-sv40NLS-Cas9-NLS T2A-mCherry
  source 1..9277: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgggtcttcgagaagacctgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 36 - DNA, 9272 bp, synthetic construct
  misc_feature 1..9272: note = Description of sequence: pBZ191-pU6-sgBFP-CBh-sv40NLS-Cas9-NLS-T2A-mCherry
  source 1..9272: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgcttttttgttttagagctagaaatagcaagttaaaataaggctagtccgtttttagcgcgtgcgccaattctgcagacaaatggctctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgct

SEQ ID NO: 37 - DNA, 11135 bp, synthetic construct
  misc_feature 1..11135: note = Description of sequence: pBZ193-pU6-sgBFP-Ht_CBh-sv 40NLS-Cas9-NLS-T2A-mCherry-P2A-Sa163RT
  source 1..11135: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtgccacctacggcaagctgaccctgaagttcatctgcaccaccggcaagctgcccgtgccctggcccaccctcgtgaccaccctgacgtacggcgtgcagtgcttcagccgctaccccgaccacatgacctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgaggcaacaaacttctcactactcaaacaagcaggtgacgtggaggagaatcccgggcctcttaagatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 38 - DNA, 11135 bp, synthetic construct
  misc_feature 1..11135: note = Description of sequence: pBZ194-pU6-sgBFP-Hn_CBh-sv 40NLS-Cas9-NLS-T2A-mCherry-P2A-Sa163RT
  source 1..11135: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagttcatgtggtcggggtagcggctgaagcactgcacgccgtacgtcagggtggtcacgagggtgggccagggcacgggcagcttgccggtggtgcagatgaacttcagggtcagcttgccgtaggtggccctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgaggcaacaaacttctcactactcaaacaagcaggtgacgtggaggagaatcccgggcctcttaagatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 39 - DNA, 11128 bp, synthetic construct
  misc_feature 1..11128: note = Description of sequence: pBZ195-pU6-sgBFP-At_CBh-sv 40NLS-Cas9-NLS-T2A-mCherry-P2A-Sa163RT
  source 1..11128: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtcctgaagttcatctgcaccaccggcaagctgcccgtgccctggcccaccctcgtgaccaccctgacgtacggcgtgcagtgcttcagccgctaccccgaccacatgaagcagcacgacttcctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgaggcaacaaacttctcactactcaaacaagcaggtgacgtggaggagaatcccgggcctcttaagatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 40 - DNA, 11128 bp, synthetic construct
  misc_feature 1..11128: note = Description of sequence: pBZ196-pU6-sgBFP-An_CBh-sv 40NLS-Cas9-NLS-T2A-mCherry-P2A-Sa163RT
  source 1..11128: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtaagtcgtgctgcttcatgtggtcggggtagcggctgaagcactgcacgccgtacgtcagggtggtcacgagggtgggccagggcacgggcagcttgccggtggtgcagatgaacttcaggcctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgaggcaacaaacttctcactactcaaacaagcaggtgacgtggaggagaatcccgggcctcttaagatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 41 - DNA, 11121 bp, synthetic construct
  misc_feature 1..11121: note = Description of sequence: pBZ197-pU6-sgBFP-Dt_CBh-sv 40NLS-Cas9-NLS-T2A-mCherry-P2A-Sa163RT
  source 1..11121: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtccctcgtgaccaccctgacgtacggcgtgcagtgcttcagccgctaccccgaccacatgaagcagcacgacttcttcaagtccgccatgcccgaaggctacgtccaggagcgccctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgaggcaacaaacttctcactactcaaacaagcaggtgacgtggaggagaatcccgggcctcttaagatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 42 - DNA, 11121 bp, synthetic construct
  misc_feature 1..11121: note = Description of sequence: pBZ198-pU6-sgBFP-Dn_CBh-sv 40NLS-Cas9-NLS-T2A-mCherry-P2A-Sa163RT
  source 1..11121: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtgcgctcctggacgtagccttcgggcatggcggacttgaagaagtcgtgctgcttcatgtggtcggggtagcggctgaagcactgcacgccgtacgtcagggtggtcacgagggcctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgaggcaacaaacttctcactactcaaacaagcaggtgacgtggaggagaatcccgggcctcttaagatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgtaggaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 43 - DNA, 11165 bp, synthetic construct
  misc_feature 1..11165: note = Description of sequence: pBZ199-pU6-sgBFP-Ht_CBh-sv 40NLS-Cas9-Sa163RT-NLS-T2A-mCherry
  source 1..11165: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtgccacctacggcaagctgaccctgaagttcatctgcaccaccggcaagctgcccgtgccctggcccaccctcgtgaccaccctgacgtacggcgtgcagtgcttcagccgctaccccgaccacatgacctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 44 - DNA, 11165 bp, synthetic construct
  misc_feature 1..11165: note = Description of sequence: pBZ200-pU6-sgBFP-Hn_CBh-sv 40NLS-Cas9-Sa163RT-NLS-T2A-mCherry
  source 1..11165: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagttcatgtggtcggggtagcggctgaagcactgcacgccgtacgtcagggtggtcacgagggtgggccagggcacgggcagcttgccggtggtgcagatgaacttcagggtcagcttgccgtaggtggccctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 45 - DNA, 11158 bp, synthetic construct
  misc_feature 1..11158: note = Description of sequence: pBZ201-pU6-sgBFP-At_CBh-sv 40NLS-Cas9-Sa163RT-NLS-T2A-mCherry
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtcctgaagttcatctgcaccaccggcaagctgcccgtgccctggcccaccctcgtgaccaccctgacgtacggcgtgcagtgcttcagccgctaccccgaccacatgaagcagcacgacttcctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 46 - DNA, 11158 bp, synthetic construct
  misc_feature 1..11158: note = Description of sequence: pBZ202-pU6-sgBFP-An_CBh-sv 40NLS-Cas9-Sa163RT-NLS-T2A-mCherry
  source 1..11158: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtaagtcgtgctgcttcatgtggtcggggtagcggctgaagcactgcacgccgtacgtcagggtggtcacgagggtgggccagggcacgggcagcttgccggtggtgcagatgaacttcaggcctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 47 - DNA, 11151 bp, synthetic construct
  misc_feature 1..11151: note = Description of sequence: pBZ203-pU6-sgBFP-Dt_CBh-sv 40NLS-Cas9-Sa163RT-NLS-T2A-mCherry
  source 1..11151: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtccctcgtgaccaccctgacgtacggcgtgcagtgcttcagccgctaccccgaccacatgaagcagcacgacttcttcaagtccgccatgcccgaaggctacgtccaggagcgccctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 48 - DNA, 11151 bp, synthetic construct
  misc_feature 1..11151: note = Description of sequence: pBZ204-pU6-sgBFP-Dn_CBh-sv 40NLS-Cas9-Sa163RT-NLS-T2A-mCherry
  source 1..11151: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacggtgcgagcgaccgagagaggtcccaagccatcagcctcagcgcctcgagcgcgagagcggcgttgcgccgctctggttgaattgcaggacactctccgcaaggtagcctgttcttggctctcttccctccggtgagtacctctccggccggggagctgaaccaacgaactagtgcgctcctggacgtagccttcgggcatggcggacttgaagaagtcgtgctgcttcatgtggtcggggtagcggctgaagcactgcacgccgtacgtcagggtggtcacgagggcctaggcgcaaccgccgtttccccggccggagaggtactcaccggaggggagagccggtgaggctaccgtgccccaggtgagaaggtggtgccttcgggcctccctcgaccgctcgcgctttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcatgacggcaaaactggaaagccacgtgcctgctgcaccaccagtctcagcggaagcgccggcaccgacgcgacctgacgctgctaagcaggaggctcgacgagcccaccatgaagccctgagactgcggtggaaagcaatcgaggaagctgggggcactgacgcctgggtacgacagcagttggttgcgaaaggcgtagcggcggaagaggtggacttcgaaagtctctccgacaagcaaaaagcggcttggaaagagaaaaaaaaagctgaggcaaccgagcggagggctcaaaaacgacttgcctgggaagcctggaaagcgacccatattcatcatttgggcgttggagttcactgggacgaagccggcgggcctgacaagttcgatgtggcagggagggaagaaagggcaaaggcaaacgggttgcccgaaggtcttgacagcgtagaagcattggcgaaagcgctgggaatctcagtatcccgattgcgctggttctcttttcacagggaagttgatacagggacacactaccaaacttgggagatcccaaagagggatgggggcaaacgcaccctcacggccccgaaaagggagcttaaggcagtacaaaggtgggtactcgccaatgtggtggaaagacttcccgtacacggagctgctcatgggtttgttgcaggacgctccatactcactaatgcgctcgctcaccaaggagctgacgtggttgttaaagttgatatgaaagatttttttcccagtgtcacatggcctcgagttaaaggcctgctcagaaagggaggtttgcctgaaaacttggcgaccctgctcgcccttctgagtaccgaagctccacgggaagtagtccgattcagaggggaaacgctgtacgtcgctaagggcccacgcgctctgccacagggcgcgcctaccagtcctgcgcttactaacgcattgtgtcttaggttggataagcggttgagcgcgctgagtaagcggttgggtttcacatacaccagatacgctgacgatcttacatttagttggaggcgcgctaaaaaatctcgccagaaagagttgccgctggctgatgctcccgtcgcgcttctcctggcccgggttaagggagtgttggaagcagaaggtttcactttgcatcctgataagaccagggttcagcggaagggaagccggcaacgagtgaccggcctcgtggttaatgaagcgcccgaaggagtgccaggtgcccgagttccccgagatgttgttcggagattgcgagcggcaatacacaatagagagcaaggtaaaccagggccgactggagaaacgttggaacaactcaaaggcttggcagcttttctgcacatgacagacgctgaaaaaggaagagcgtttctgagaagacttgaggccctggagaagaggcaaactgcgaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgt

SEQ ID NO: 49 - DNA, 10491 bp, synthetic construct
  misc_feature 1..10491: note = Description of sequence: pBZ207-pU6-sgBFP-Ht_CBh-sv 40NLS-Cas9-Ec86RT-NLS-T2A-mCherry
  source 1..10491: mol_type = other DNA
catgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacgatgcgcacccttagcgagaggtttatcattaaggtcaacctctggatgttgtttcggcatcctgcattgaatctgagttactgtctgtttccctactagtgccacctacggcaagctgaccctgaagttcatctgcaccaccggcaagctgcccgtgccctggcccaccctcgtgaccaccctgacgtacggcgtgcagtgcttcagccgctaccccgaccacatgacctaggagggaacccgtttcttctgacgtaagggtgcgcatttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggc

SEQ ID NO: 50 - DNA, 10491 bp, synthetic construct
  misc_feature 1..10491: note = Description of sequence: pBZ208-pU6-sgBFP-Hn_CBh-sv 40NLS-Cas9-Ec86RT-NLS-T2A-mCherry
  source 1..10491: mol_type = other DNA
catgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacgatgcgcacccttagcgagaggtttatcattaaggtcaacctctggatgttgtttcggcatcctgcattgaatctgagttactgtctgtttccctactagttcatgtggtcggggtagcggctgaagcactgcacgccgtacgtcagggtggtcacgagggtgggccagggcacgggcagcttgccggtggtgcagatgaacttcagggtcagcttgccgtaggtggccctaggagggaacccgtttcttctgacgtaagggtgcgcatttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggc

SEQ ID NO: 51 - DNA, 10484 bp, synthetic construct
  misc_feature 1..10484: note = Description of sequence: pBZ209-pU6-sgBFP-At_CBh-sv 40NLS-Cas9-Ec86RT-NLS-T2A-mCherry
  source 1..10484: mol_type = other DNA
catgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacgatgcgcacccttagcgagaggtttatcattaaggtcaacctctggatgttgtttcggcatcctgcattgaatctgagttactgtctgtttccctactagtcctgaagttcatctgcaccaccggcaagctgcccgtgccctggcccaccctcgtgaccaccctgacgtacggcgtgcagtgcttcagccgctaccccgaccacatgaagcagcacgacttcctaggagggaacccgtttcttctgacgtaagggtgcgcatttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggc

SEQ ID NO: 52 - DNA, 10484 bp, synthetic construct
  misc_feature 1..10484: note = Description of sequence: pBZ210-pU6-sgBFP-An_CBh-sv 40NLS-Cas9-Ec86RT-NLS-T2A-mCherry
  source 1..10484: mol_type = other DNA
catgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacgatgcgcacccttagcgagaggtttatcattaaggtcaacctctggatgttgtttcggcatcctgcattgaatctgagttactgtctgtttccctactagtaagtcgtgctgcttcatgtggtcggggtagcggctgaagcactgcacgccgtacgtcagggtggtcacgagggtgggccagggcacgggcagcttgccggtggtgcagatgaacttcaggcctaggagggaacccgtttcttctgacgtaagggtgcgcatttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggc

SEQ ID NO: 53 - DNA, 10477 bp, synthetic construct
  misc_feature 1..10477: note = Description of sequence: pBZ211-pU6-sgBFP-Dt_CBh-sv 40NLS-Cas9-Ec86RT-NLS-T2A-mCherry
  source 1..10477: mol_type = other DNA
catgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacgatgcgcacccttagcgagaggtttatcattaaggtcaacctctggatgttgtttcggcatcctgcattgaatctgagttactgtctgtttccctactagtccctcgtgaccaccctgacgtacggcgtgcagtgcttcagccgctaccccgaccacatgaagcagcacgacttcttcaagtccgccatgcccgaaggctacgtccaggagcgccctaggagggaacccgtttcttctgacgtaagggtgcgcatttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggc

SEQ ID NO: 54 - DNA, 10477 bp, synthetic construct
  misc_feature 1..10477: note = Description of sequence: pBZ212-pU6-sgBFP-Dn_CBh-sv 40NLS-Cas9-Ec86RT-NLS-T2A-mCherry
  source 1..10477: mol_type = other DNA
catgtgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccgctgaagcactgcacgccatgttttagagctagaaatagcaagttaaaataaggctagtccgttatcaacttgaaaaagtggcaccgagtcggtgccgtacgatgcgcacccttagcgagaggtttatcattaaggtcaacctctggatgttgtttcggcatcctgcattgaatctgagttactgtctgtttccctactagtgcgctcctggacgtagccttcgggcatggcggacttgaagaagtcgtgctgcttcatgtggtcggggtagcggctgaagcactgcacgccgtacgtcagggtggtcacgagggcctaggagggaacccgtttcttctgacgtaagggtgcgcatttttttctagaggtacccgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcaggttggaccggtgccaccatggactataaggaccacgacggagactacaaggatcatgatattgattacaaagacgatgacgataagatggccccaaagaagaagcggaaggtcggtatccacggagtcccagcagccgacaagaagtacagcatcggcctggacatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgactctggaggatctagcggaggatcctctggcagcgagacaccaggaacaagcgagtcagcaacaccagagagcagtggcggcagcagcggcggcagcagcaagagtgcggaataccttaacacatttaggcttagaaacctgggactccccgttatgaataacctgcacgatatgtctaaggcaacccgaatcagtgtagagacgcttagactcttgatatataccgccgactttcgatacagaatatataccgtcgagaagaagggcccagagaaacgaatgcggaccatataccaacctagtagggagctgaaagcgcttcagggctgggtacttcgaaatatccttgacaaactttccagttcaccgttcagcattggtttcgagaaacaccagagtattctgaataacgcgacacctcacataggagccaatttcatcctcaatattgacctggaggatttcttccctagccttactgccaataaagtgttcggtgtattccacagcctcggctacaaccgactgatttcatctgtactcacaaaaatttgttgctataagaacctgctccctcaaggggccccaagtagcccaaaactggcaaacctcatctgttcaaaattggactatcgaattcagggctatgccggatccaggggcttgatctacacgagatacgcagacgacctgacactttcagcacaatccatgaagaaagttgttaaagcgagggattttcttttttccattattccgtctgaaggattggttattaattctaaaaaaacttgcattagtggtcctcggtctcaacgaaaggttacaggcctggtaatctctcaggagaaggttgggattggaagagagaagtataaagagattcgcgccaaaatacatcatattttttgcggtaaatcatctgaaatcgagcacgtaaggggatggctttctttcattctttctgtagactccaaatctcaccgacgactcatcacatatataagtaaactggagaaaaaatatgggaaaaatccgcttaacaaggctaaaactaaaaggccggcggccacgaaaaaggccggccaggcaaaaaagaaaaagcttgagggcagaggaagtctgctaacatgcggtgacgtggaggagaatcccggccctgctagcatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtgagaattcctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagagaatagcaggcatgctggggagcggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtggaagccgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggc

SEQ ID NO: 55 - DNA, 249 bp, synthetic construct
  misc_feature 1..249: note = Description of sequence: RNA polymerase III promoter U6
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 56 - DNA, 794 bp, synthetic construct
  misc_feature 1..794: note = Description of sequence: RNA polymerase II promoter CBh
  source 1..794: mol_type = other DNA
cgttacataacttacggtaaatggcccgcctggctgaccgcccaacgacccccgcccattgacgtcaatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattgtgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagctgagcaagaggtaagggtttaagggatggttggttggtggggtattaatgtttaattacctggagcacctgcctgaaatcactttttttcag

SEQ ID NO: 57 - DNA, 48 bp, synthetic construct
  misc_feature 1..48: note = Description of sequence: Nucleoplasmin nuclear localization sequence (NLS)
  source 1..48: mol_type = other DNA
aaaaggccggcggccacgaaaaaggccggccaggcaaaaaagaaaaag

SEQ ID NO: 58 - DNA, 21 bp, synthetic construct
  misc_feature 1..21: note = Description of sequence: Simian virus 40 (SV40) nuclear localization sequence (NLS)
  source 1..21: mol_type = other DNA
ccaaagaagaagcggaaggtc

SEQ ID NO: 59 - RNA, 82 nt, synthetic construct
  misc_feature 1..82: note = Description of sequence: sgRNA scaffold sequence
  source 1..82: mol_type = other RNA
gttttagagctagaaatagcaagttaaaataaggctagtccgttatcaacttgaaaaagtggcaccgagtcggtgctttttt

SEQ ID NO: 60 - RNA, 22 nt, synthetic construct
  misc_feature 1..22: note = Description of sequence: crRNA repeat sequence
  source 1..22: mol_type = other RNA
gttttagagctatgctgttttg

SEQ ID NO: 61 - RNA, 85 nt, synthetic construct
  misc_feature 1..85: note = Description of sequence: Native tracrRNA sequence
  source 1..85: mol_type = other RNA
ggaaccattcaaaacagcatagcaagttaaaataaggctagtccgttatcaacttgaaaaagtggcaccgagtcggtgctttttt

SEQ ID NO: 62 - protein, 20 aa, synthetic construct
  REGION 1..20: note = Description of sequence: E2A self-cleaving peptide
QCTNYALLKLAGDVESNPGP

SEQ ID NO: 63 - protein, 18 aa, synthetic construct
  REGION 1..18: note = Description of sequence: T2A self-cleaving peptide
EGRGSLLTCGDVEENPGP

SEQ ID NO: 64 - protein, 19 aa, synthetic construct
  REGION 1..19: note = Description of sequence: P2A self-cleaving peptide
ATNFSLLKQAGDVEENPGP

SEQ ID NO: 65 - protein, 22 aa, synthetic construct
  REGION 1..22: note = Description of sequence: F2A self-cleaving peptide
VKQTLNFDLLKLAGDVESNPGP